(19) United States
(12) Patent Application Publication
(10) Pub. No.: US 2011/0183374 A1
(43) Pub. Date: Jul. 28, 2011

(54) THIOPEPTIDE PRECURSOR PROTEIN, GENE ENCODING IT AND USES THEREOF

(75) Inventor: Rowan Morris, Basel (CH)

(73) Assignee: NOVARTIS AG

(21) Appl. No.: 12/672,379

(22) PCT Filed: Aug. 6, 2008
(86) PCT No.: PCT/EP08/60339

§ 371 Date: Feb. 5, 2010

(30) Foreign Application Priority Data
Aug. 9, 2007 (EP) 07114121.2

(51) Int. Cl. C07K 7/08; C07H 21/04; C12N 15/63; C07K 14/37; C12N 1/15; C12N 1/21; C12P 21/00

(52) U.S. Cl. 435/69.1; 530/327; 530/326; 536/23.74; 435/320.1; 530/350; 435/254.11; 435/252.33; 435/252.3; 435/252.31; 435/252.35

(57) ABSTRACT

The present invention relates to the precursor proteins for thiopeptide biosynthesis, and the corresponding structural genes and uses thereof. The present invention also relates to methods for genetically manipulating the thiopeptide precursor protein or host cells expressing a gene encoding said thiopeptide precursor protein to produce thiopeptide compounds or their derivatives. The present invention further relates to the cloning and characterization of genes involved in thiopeptide biosynthesis and their use in thiopeptide compounds production.

The present invention relates to the precursor proteins for thiopeptide biosynthesis, and the corresponding structural genes and uses thereof. The present invention also relates to methods for genetically manipulating the thiopeptide precursor protein or host cells expressing a gene encoding said thiopeptide precursor protein to produce thiopeptide compounds or their derivatives. The present invention further relates to the cloning and characterization of genes involved in thiopeptide biosynthesis and their use in thiopeptide compounds production.

The thiopeptides are naturally occurring, sulphur-rich, highly modified, macrocyclic peptides, many of which have potent antibiotic activity. These complex natural products are grouped as thiazolyl peptides according to Berdy's chemical classification of antibiotics (Berdy J. ‘Recent developments of antibiotic research and classification of antibiotics according to chemical structure. ‘Adv Appl Microbiol. 1974; 18(0):309-406.) and include thiostrepton, nosiheptide, micrococcin, nocathiacin, amythiamicin, GE2270A etc. The thiopeptides share a number of common structural features: a tri- or tetrasubstituted nitrogen heterocycle clustered in a central polyazole domain that is part of a macrocyclic framework consisting of modified heterocyclic residues, including thiazoles, oxazoles, and indoles, and dehydroamino acids. It is this macrocyclic signature of the thiopeptides that distinguishes them from other thiazolyl compounds, such as bleomycin, bacitracin, or microcin B17. It was Hensens who first suggested grouping thiopeptide antibiotics according to the structure and oxidation state of the central heterocyclic domain (O. D. Hensens and G. Albers-Schoenberg. ‘Total structure of the peptide antibiotic components of thiopeptin by ¹H and ¹³C NMR spectroscopy.’ Tetrahedron Lett. 1978, 3649). A recent review by Mark Bagley and colleagues from Cardiff University has extended Hensens’ classification system to describe five distinct central heterocyclic domains: tetrasubstituted dehydropiperidine or saturated piperidine, dihydroimidazopiperidine, trisubstituted pyridine and tetrasubstituted hydroxypyridine (Bagley et al. ‘Thiopeptide antibiotics.’ Chem. Rev. 2005 February; 105(2):685-714).

Despite considerable structural homology, the sites and modes of action (inhibition of protein synthesis) of the thiopeptide antibiotics can be categorized into two functional classes: those that bind to a region of the 23S ribosomal RNA known as the L11 binding domain and those that bind to the Ef-Tu protein complex involved in the elongation cycle. Thiostrepton, which was first isolated in 1954 as a secondary metabolite of Streptomyces azureus, is often regarded as the archetypal compound of the thiopeptide family (Donovick et al. ‘Thiostrepton, a new antibiotic.’ Antibiot Annu. 1955-1956; 3:554-9 and Bagley et al. ‘Thiopeptide antibiotics.’ Chem. Rev. 2005 February; 105(2):685-714).

It was postulated that thiopeptides are synthesized by a nonribosomal process that utilizes large, multi-enzyme complexes, termed nonribosomal peptide synthetases (NRPS) (Mocek et al. ‘Biosynthesis of the modified peptide antibiotic thiostrepton in Streptomyces azureus and Streptomyces laurentii’. J. Am. Chem. Soc. 1993, 115, 7992-8001). An example of an NRPS-assembled macrocycle is the immunosuppressant peptide cyclosporine which is produced by the fungus Tolypocladium inflatum. NRPS-assembled peptides are not restricted to the common 20 L-amino acids typically found in proteins. Instead these enzymes have the biosynthetic capability of incorporating a broader repertoire of hundreds of unusual amino acids and derived residues.

Preliminary experiments conducted by Heinz Floss et al., University of Washington, suggest that the thiopeptides are indeed produced by a nonribosomal pathway. This group treated cultures with chloramphenicol at levels that inhibited ribosomal protein synthesis. However, de novo production of thiopeptide continued. Oligonucleotides derived from the amino acid sequence representing either thiostrepton or the related nosiheptide failed to hybridize to any homologous sequences when used to probe Southern blots of genomic DNA from the respective producing organisms (T. M. Smith, Y.-F. Jiang, H. G. Floss. ‘Thiopeptide Antibiotics” in “Biotechnology of Antibiotics” W. R. Strohl, Ed., 2nd Ed., Marcel-Dekker, New York, 1997, pp 393-413).

Additionally, a NRPS gene fragment that is postulated to encode a module of the micrococcin synthetase complex has been identified in the producing strain (Carnio et al. ‘Pyridinyl polythiazole class peptide antibiotic micrococcin P1, secreted by foodborne Staphylococcus equorum WS2733, is biosynthesized nonribosomally.’ Eur J. Biochem. 2001 December; 268(24):6390-401.).

However, to date there has been no published description of a thiopeptide biosynthetic pathway.

Therefore, there is a need to identify genes involved in thiopeptide biosynthesis that will provide tools for efficient thiopeptide production and possibilities of generating alternative thiopeptide structures. The present invention fulfills this need and discloses for the first time a chromosomally encoded backbone for thiopeptide biosynthesis, as well as the core biosynthetic enzymes involved in thiopeptide biosynthesis.

### 1. A Chromosomally Encoded Backbone as Starting Material for Thiopeptide Biosynthesis

The inventors have indeed found clear evidence that a chromosomally encoded backbone is the starting material for thiopeptide biosynthesis, for example in actinomycete species.

In a first aspect, the invention relates to a thiopeptide precursor protein comprising an amino acid sequence selected among the group consisting of

### (i) SEQ ID NO:1;

### (ii) SEQ ID NO: 5;

(iii) SEQ ID NO:11; and
(iv) a variant of any of said amino acid sequences.

As used herein, the term “thiopeptide” refers to thiazolyl peptides according to Berdy's chemical classification of antibiotics that are characterized, contrary to other thiazolyl compounds, such as bleomycin, bacitracin, or microcin B17, by a tri- or tetrasubstituted nitrogen heterocycle clustered in a central polyazole domain that is part of a macrocyclic framework consisting of modified heterocyclic residues, including thiazoles, oxazoles, and indoles, and dehydroamino acids.

The term “thiopeptide precursor protein” refers to a gene encoded polypeptide backbone which could be used as a starting material for in vitro or in vivo thiopeptide synthesis. Preferably, said thiopeptide precursor is a precursor for an inhibitor of the bacterial elongation factor Ef-tu. Ef-tu inhibitors are described for example in the review by Hogg T, Mesters J R and Hilgenfeld R. (‘Inhibitory mechanisms of antibiotics targeting elongation factor Tu.’ Curr Protein Pept Sci. 2002 February; 3(1):121-31). Examples of well-known Ef-tu inhibitors are GE2270A (as defined in Selva et al. ‘Antibiotic GE2270A: a novel inhibitor of bacterial protein synthesis. I. Isolation and characterization.’ J Antibiot (Tokyo). 1991 July; 44(7):693-701.), GE37648A (as defined in Stella et al. ‘Antibiotic GE37468 A: a new inhibitor of bacterial protein synthesis. I. Isolation and characterization.’ J Antibiot (Tokyo). 1995 August; 48(8):780-6. Erratum in: J Antibiot (Tokyo) 1995 December; 48(12):C-3.), and Amythiamicin (as defined in ('Novel antibiotics, amythiamicins.’ J Antibiot (Tokyo). 1994 June; 47(6):668-74, 1145-52 and 1153-9.). Novel Ef-tu inhibitors are also described in International Patent Application Number PCT/US07/025,955 filed on 19 Dec. 2007. Such novel Ef-tu inhibitors include more specifically compounds of formulas I through XI (including pharmaceutically acceptable salts thereof, as well as enantiomers, stereoisomers, rotamers, tautomers, diastereomers, or racemates thereof) as represented below:

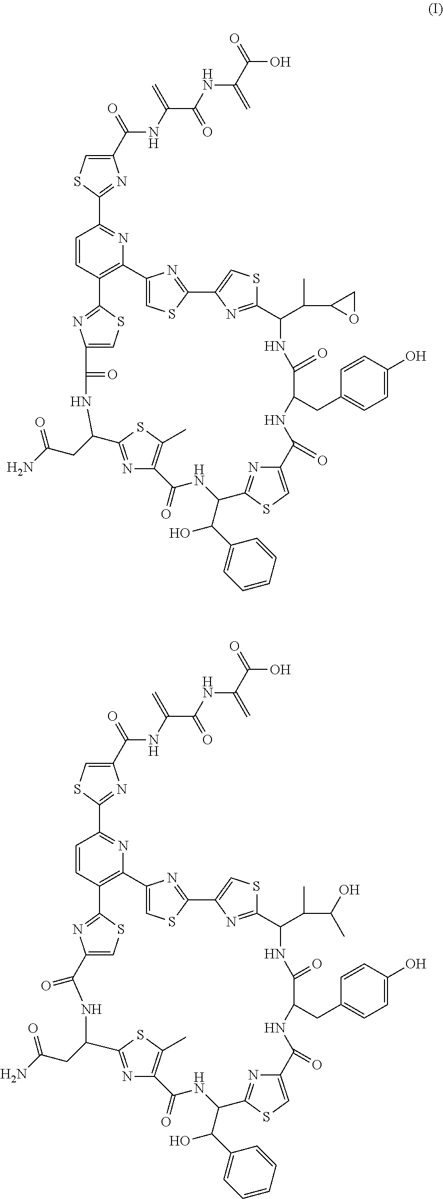

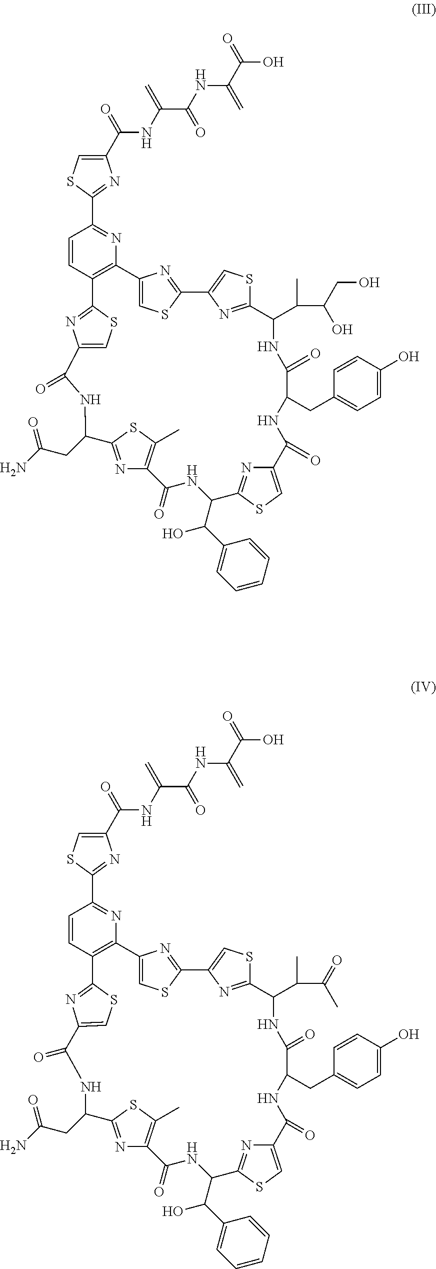

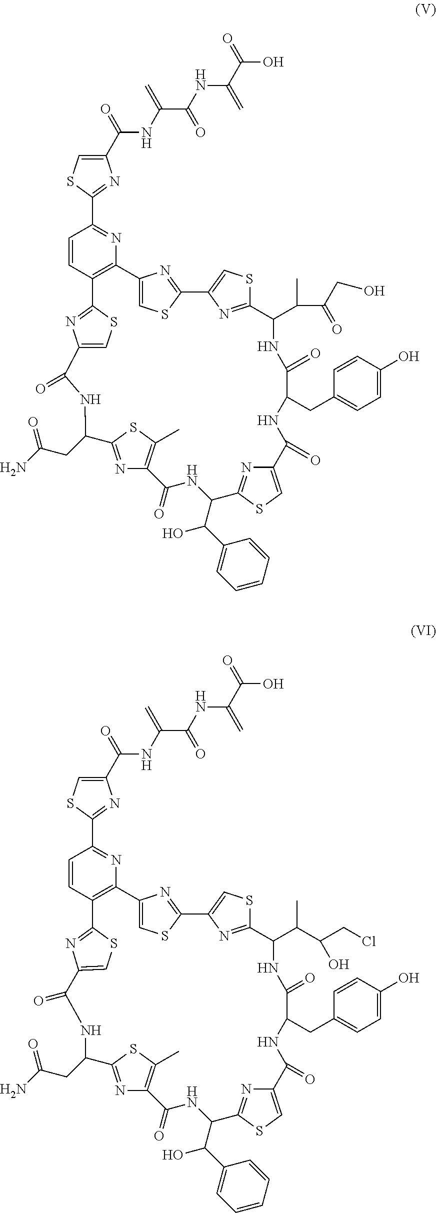

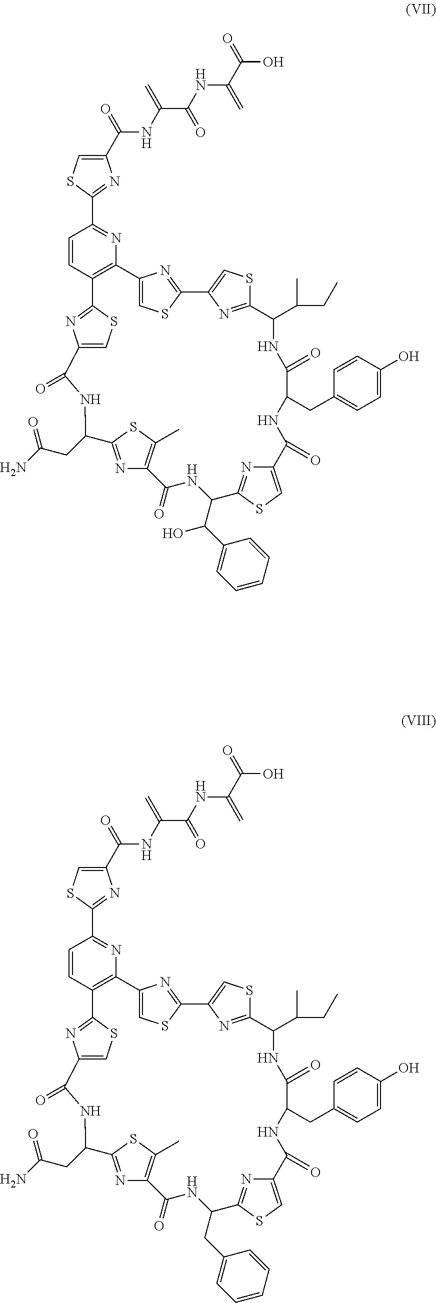

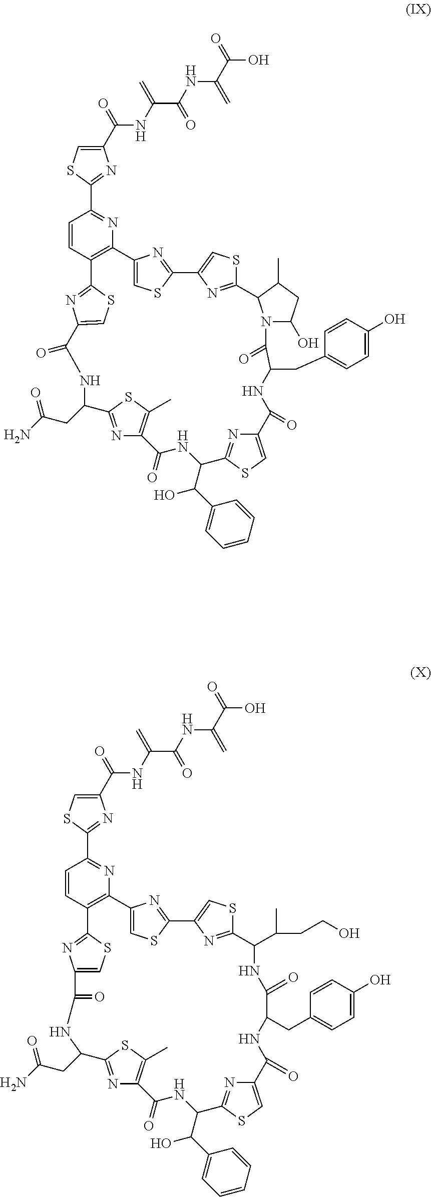

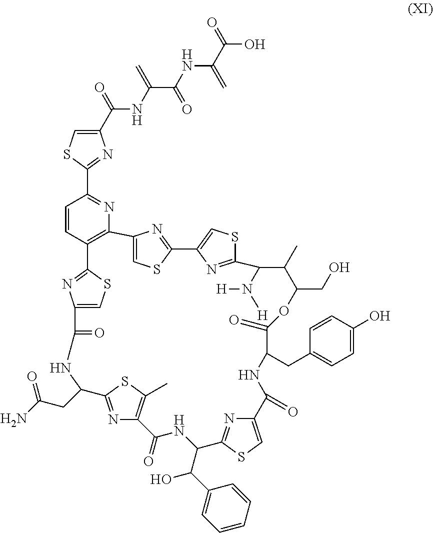

The amino acid sequence of such precursor protein is critical for the final structure of the thiopeptide. As this will be described below in detail, the thiopeptide precursor proteins of the invention can be used to reproduce the backbone of a newly identified thiopeptide or to generate novel derivatives of known thiopeptides, for example with improved properties.

The invention therefore not only relates to the precursor protein of SEQ ID NO:1, SEQ ID NO: 5 or SEQ ID NO: 11 but also to any variant which could be useful for biosynthesis of thiopeptide derivatives. The one skilled in the art will know how to design said variant depending on the amino acid backbone of the thiopeptide to be synthesized. In SEQ ID NO:1, the six cysteines found within the 14 amino-acid backbone are the precursors of the thiazole heterocycles that decorate these molecules. In addition, two serine residues are involved in the formation of the pyridine ring system at the juncture of the macrocycle and the side-chain. In the thiopeptide side-chain of the GE2270A molecule is found an additional oxazoline and in the novel Ef-tu inhibitors of formulas (I) to (XI) are found dehydroalanine residues formed by the heterocyclization and dehydration of serine amino-acids respectively.

In a specific embodiment, a variant of SEQ ID NO:1, SEQ ID NO 5 or SEQ ID NO 11 has no more than 1, 2, 3, 4, 5, 6 or 10 deleted, inserted or substituted amino acids when compared to original sequence. Substituted amino acids can be either natural amino acid with equivalent functional group or with different functional group or non-natural amino acid.

The following table 1 gives an indication of the thiopeptide precursor protein sequence (SEQ ID NO:1-14) that can be used for biosynthesis of corresponding specific thiopeptides.

TABLE 1
Primary sequence alignment of
thiopeptide amino acid backbones.
The alignment is centered around the
macrocycle that is bounded by two
invariant serine molecules that
form the signature multi-substituted
nitrogen heterocycle. Thiostrepton
has an additional amino extension of
4 amino acids.

| | Primary Amino Acid Sequence | |
|---|---|---|
| Thiopeptide | | Side Chain |
| | 11 aa Macrocycle | |
| GE2270A | SCNCVCGFCCS | CSP (NO: 1) |
| GE37468A | SSNCFCYPCCS | CSS (NO: 2) |
| Novel EF Tu | SCNCFCYICCS | CSS (NO: 3) |
| inhibitors | | |
| Amythiamicin | SCNCVCGVCCS | CSP (NO: 4) |
| | 10 aa Macrocycle | |
| Micrococcin | SCTTCVCTCS | CCT (NO: 5) |
| Thiostrepton | IASA SCTTCICTCS | CSS (NO: 6) |
| Nosiheptide | SCTTCECCCS | CS (NO: 7) |
| Thiocillin | SCTTCVCVCS | CCT (NO: 8) |
| Thiocins | SCVGSACASS | SSS (NO: 9) |
| Nocathiacins | SCTTCXCXCS | CS (NO: 10) |
| | 13 aa Macrocycle | |
| Berninamycin | SCTTSSVSSSSSS | SS (NO: 11) |
| Sulfomycins | SCTTSGCTSSSSS | SSS (NO: 12) |
| A10255B | SCTTSGSACSSSS | SSSS (NO: 13) |
| Radamycin | SCVGSACACSSSS | SS (NO: 14) |

The following standard code letter is used in the instant application for describing any amino acid, peptide and protein sequence: A, Alanine; R, Arginine; N, Asparagine; D, Aspartic acid; C, Cysteine; Q, Glutamine; E, Glutamic acid; G, Glycine; H, Histidine; I, Isoleucine; L, Leucine; K, Lysine; M, methionine; F, phenylalanine; P, Proline; S, Serine; T, Threonine; W, Tryptophane; Y, Tyrosine; V, Valine.

More generally, in one specific embodiment, said variant of amino acid sequence SEQ ID NO:1 has the following formula: SXNCXCXXCCSCSX, wherein X can be any amino acid. More preferably, said variant of SEQ ID NO:1 comprises the following formula: SX₁NCX₂CX₃X₄CCSCSX₅, wherein X₁ is C or S, X₂ is V or F, X₃ is G or Y, X₄ is a F, P, I or V and X₅ is P or S. Examples of such variants are the precursor proteins of SEQ ID NO:2-4.

In another specific embodiment, said variant of amino acid sequence SEQ ID NO:5 comprises the following formula: SCX₁X₂X₃CX₄CX₅X₆X₇X₈ wherein X₁ is T or V, preferably T, X₂ is T or G, preferably T, X₃ is C or S, preferably C, X₄ is any amino acid, preferably V, I, E or A, X₅ is any amino acid, preferably T, C, V or A, X₆ and X₇ is, independently C or S, preferably C, X₈ is C or S, and X₉ is either no amino acid residue or T or S. Examples of such variants are the precursor protein of SEQ ID NO:6-10.

In another specific embodiment, said variant of amino acid sequence SEQ ID NO:11 comprises the following formula: SCX₁X₂SX₃X₄X₅X₆SSSSSS wherein X₁ is T or V, preferably T, X₂ is T or G, preferably T, X₃ is S, G or A, X₄ is V, C or A, X₅ is S, T or A, X₆ is C or S. Examples of such variants are the precursor protein of SEQ ID NO:12-14.

### 2. Nucleic Acids of Genes Encoding the Thiopeptide Precursor Protein

The invention further provides nucleic acids of genes and/or open reading frames encoding the thiopeptide precursor protein. More specifically, the invention provides a nucleic acid comprising the nucleotide sequence encoding the thiopeptide precursor protein as defined above.

A “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes.

A “recombinant DNA molecule” is a DNA molecule that has undergone a molecular biological manipulation.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, other ribosome binding site sequences, regulatory response elements, signal sequences, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.).

The term “gene”, also called a “structural gene” means a DNA sequence that codes for or corresponds to a particular sequence of amino acids which comprise all or part of one or more proteins, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG or GTG) and a stop codon.

A coding sequence is “under the control of” or “operatively associated with” expression control sequences in a cell when RNA polymerase transcribes the coding sequence into RNA, particularly mRNA, and translated into the protein encoded by the coding sequence.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. For example, the present invention includes chimeric DNA molecules that comprise a DNA sequence and a heterologous DNA sequence that is not part of the DNA sequence. In this context, the heterologous DNA sequence refers to a DNA sequence that is not naturally located within the thiopeptide biosynthetic gene cluster sequence. Alternatively, the heterologous DNA sequence can be naturally located within the biosynthetic gene cluster at a location where it does not natively occur. A heterologous expression regulatory element is an element operatively associated with a different gene than the one it is operatively associated with in nature. In the context of the present invention, a gene encoding a protein of interest is heterologous to the vector DNA in which it is inserted for cloning or expression, and it is heterologous to a host cell containing such a vector, in which it is expressed.

The term “expression control sequence” refers to a promoter, any enhancer element, or suppression elements (e.g., an origin of replication) that combine to regulate the transcription of a coding sequence. The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transformation” means the introduction of a “foreign” (i.e. extrinsic or extracellular) gene, DNA or RNA sequence to a cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cells genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The inventors were successful in identifying small structural genes in the genomes of Ef-tu inhibitor thiopeptide-producing Nomomuraea species, which encoded the whole peptide backbone of SEQ ID NO:1 and SEQ ID NO:3. The predicted size of the precursor preproproteins are 57 and 49 amino-acids respectively and are depicted in SEQ ID NO:19 and SEQ ID NO:20 respectively. However, there are a number of initiation codons that may indicate alternative translation start sites. The 14 amino-acid thiopeptide precursor sequence is at the C-terminus.

A similar structural gene for the unrelated thiopeptide thiostrepton has also been identified from Streptomyces azureus ETH28555 sp. as shown in the example 4 and which encodes the whole peptide backbone of SEQ ID NO:6. The predicted size of the precursor preproprotein for thiostrepton is 60 amino acids and is depicted in SEQ ID NO:65.

In one embodiment, said nucleic acid of the invention comprises the nucleotide sequence of SEQ ID NO:15 encoding the 14 amino acids thiopeptide precursor of SEQ ID NO:1. In another embodiment, said nucleic acid of the invention comprises the nucleotide sequence of SEQ ID NO:16 encoding the 14 amino acids thiopeptide precursor of SEQ ID NO:3. The invention further includes any nucleotide sequence encoding the thiopeptide precursor of SEQ ID NO:1, SEQ ID NO:5 and SEQ ID NO:11 and any variant thereof, such as those precursor variants depicted in Table 1. Examples of such nucleotide sequences are depicted in SEQ ID NO:17 and 18 or SEQ ID NO:65, encoding respectively thiopeptide precursor protein for SEQ ID NO:1 and SEQ ID NO:3 and SEQ ID NO:6.

Methods to isolate and identify such nucleotide sequence of the invention from known thiopeptide producing strain starting from probes or primers derived of SEQ ID NO:15-18 are well known in the art and one example of such method is shown in Example 4. For example, genomic DNA of a thiopeptide producing strain can be amplified using primers flanking the region encoding the 14 amino acid precursor protein and determined from SEQ ID NO:17 or SEQ ID NO:18 as disclosed herein.

In another embodiment, said nucleic acid of the invention comprises the nucleotide sequence of SEQ ID NO: 17 or SEQ ID NO:18 encoding the 57 amino acid or 49 amino acid preproprotein respectively, or any fragment thereof comprising at least SEQ ID NO:15 or SEQ ID NO:16.

Also included are modifications of said nucleic acids. Such modifications include, for example, labels which are known in the art, methylation, and substitution of one or more of the naturally occurring nucleotides with a degenerate nucleotide. These modifications may be to increase expression, yield, and/or to improve purification in the selected expression systems, or for another desired purpose.

In one embodiment, the nucleic acid of the invention is operatively linked to heterologous transcriptional and translational control sequence. More preferably, it is an expression vector.

As used herein, the terms “expression vector” refer to the vehicle by which a nucleic acid can be introduced into a host cell, resulting in expression of the introduced sequence. In one embodiment, vectors comprise a promoter and one or more control elements (e.g., enhancer elements) that are heterologous to the introduced nucleic acid but are recognized and used by the host cell. In another embodiment, the sequence that is introduced into the vector retains its natural promoter that may be recognized and expressed by the host cell. In one embodiment, the vectors compatible with the present invention are the shuttle vectors pSET152, pOJ436, pOJ446 (Bierman et al. ‘Plasmid cloning vectors for the conjugal transfer of DNA from Escherichia coli to Streptomyces spp.’ Gene. 1992 Jul. 1; 116(1):43-9), pHM11a (Motamedi et al. ‘Integrative vectors for heterologous gene expression in Streptomyces spp.’ Gene. 1995 Jul. 4; 160(1):25-31) and pIJ8600 (Sun et al. ‘Green fluorescent protein as a reporter for spatial and temporal gene expression in Streptomyces coelicolor A3(2).’ Microbiology. 1999 September; 145 (Pt 9):2221-7) and their derivatives. In another embodiment, the vector is a cosmid.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

A common type of vector is a “plasmid”, which generally is a self-contained molecule of double-stranded DNA (which may be circular), usually of bacterial origin, that can readily accept additional (foreign) DNA and which can readily be introduced into a suitable host cell.

A plasmid vector often contains coding DNA and promoter DNA and has one or more restriction sites suitable for inserting foreign DNA. Recombinant cloning vectors will often include one or more replication systems for cloning or expression, one or more markers for selection in the host, e.g. antibiotic resistance, and one or more expression cassettes.

Vector constructs may be produced using conventional molecular biology and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); F. M. Ausubel et al. (eds.), Current Protocols in Molecular Biology, John Wiley & Sons, Inc. (1994). Alternatively, vector constructs could be partially or fully synthesized using DNA synthesis technology of a constructive biology company such as CodonDevices (Cambridge, Mass., USA; http://www.codondevices.com/) or Blue Heron Biotechnology (Bothwell, Wash., USA; http://www.blueheronbio.com/).

3. Core Biosynthetic Enzymes for Producing Core Macrocycles from Thiopeptide Precursor

The characterization of two thiopeptide biosynthetic gene clusters from two distinct thiopeptide producing strains allows the inventors to identify genes that are highly conserved among the two strains, therefore, suggesting that these genes encode enzymes required for the synthesis of a core thiopeptide molecule from the thiopeptide precursor. FIGS. 2 and 3 show the position of Open Reading Frames (ORFs) of the biosynthetic gene cluster characterized from two distinct thiopeptide producing strains.

In another aspect, the invention relates to a polypeptide for the biosynthesis of thiopeptide, comprising an amino acid sequence selected among the group consisting of:

### (i) any one of SEQ ID NO:23-34; and.

(ii) a variant of an amino acid sequence listed in (i), having no more than 1, 2, 3, 4, 5, 6 or 10 deleted, inserted or substituted amino acids when compared to the corresponding wild type amino acid sequence listed in (i).

These polypeptides may be used either in vitro or in vivo for performing one or more reaction steps using the thiopeptide precursor protein as a starting material for the synthesis of a thiopeptide molecule. The variant polypeptide may retain substantially the same catalytic activity as the wild type corresponding sequence or have improved or modified catalytic activity. Solely for ease of reading, these polypeptides will be referred as the “core biosynthetic enzymes” in the text hereafter.

The following table 2 describes examples of core biosynthetic enzymes (SEQ ID NO:23-34) that can be used for thiopeptide biosynthesis and their proposed function in the biosynthetic pathway. The proposed function is further detailed below in Examples 5.1 and 5.2.

TABLE 2
Examples of core enzymes for thiopeptide biosynthesis
starting from thiopeptide precursor protein

| | Polypeptide | Nt | Coordinates of | Homology and |
|---|---|---|---|---|
| ORF# | SEQ ID | SEQ ID | genomic BAC | Proposed function |
| ORF9-I | NO: 23 | NO: 35 | 9646(GTG)-12129(TGA) | Lantibiotic dehydratase/ |
| | | | of NO: 62 | Serine → |
| ORF6-II | NO: 29 | NO: 41 | 4866(TTG)→7479(TGA) | Dehydroalanine |
| | | | of NO: 63 | |
| ORF10-I | NO: 24 | NO: 36 | 12126(ATG)-13025(TGA) | Lantibiotic dehydratase/ |
| | | | of NO: 62 | Serine → |
| ORF7-II | NO: 30 | NO: 42 | 7475(ATG)→8291(TGA) | Dehydroalanine |
| | | | of NO: 63 | |
| ORF11-I | NO: 25 | NO: 37 | 13025(ATG)-14026(TGA) | Lantibiotic biosynthesis |
| | | | of NO: 62 | |
| ORF8-II | NO: 31 | NO: 43 | 8287(ATG)→9247(TGA) | |
| | | | of NO: 63 | |
| ORF12-I | NO: 26 | NO: 38 | 14023(GTG)-15390(TGA) | Thiazoline oxidase/ |
| | | | of NO: 62 | Subtilisin-like protease |
| ORF9-II | NO: 32 | NO: 44 | 9243(ATG)→10530(TGA) | |
| | | | of NO: 63 | |
| ORF13-I | NO: 27 | NO: 39 | 15378(ATG)-17204(TGA) | Conserved hypothetical |
| | | | of NO: 62 | protein |
| ORF10-II | NO: 33 | NO: 45 | 10526(TTG)→12419(TGA) | |
| | | | of NO: 63 | |
| ORF14-I | NO: 28 | NO: 40 | 17201(ATG)-18937(TGA) | Adenylation/ |
| | | | of NO: 62 | heterocyclisation |
| ORF11-II | NO: 34 | NO: 46 | 12411(ATG)→14256(TGA) | |
| | | | of NO: 63 | |

### 4. Genes Encoding the Core Biosynthetic Enzymes

The invention further provides nucleic acids of genes and/or open reading frames encoding the core biosynthetic enzymes. More specifically, the invention provides a nucleic acid comprising the nucleotide sequence encoding any one of the core biosynthetic enzymes as defined above.

The inventors were successful in identifying the structural genes in the genomes of Ef-tu inhibitor thiopeptide producing Nomomuraea species, which likely encode essential enzymes for the biosynthesis of corresponding Ef-tu inhibitors.

In one embodiment, said nucleic acid encoding an enzyme for the biosynthesis of Ef-tu inhibitors comprises a nucleic acid selected among the group consisting of ORF9, ORF10, ORF11, ORF12, ORF13 and ORF15 of the genomic fragment of SEQ ID NO:62 as described in Table 2. In another embodiment, said nucleic acid of the invention comprises a nucleic acid selected among the group consisting of ORF6, ORF7, ORF8, ORF9, ORF10 and ORF11 of the genomic fragment of SEQ ID NO:63 as described in Table 2. The nucleotide position (coordinates) of the ORF (Open Reading Frame) of each gene in the corresponding genomic fragment (from 5′ to 3′) are also reported in Table 2.

Methods to isolate and identify such nucleotide sequence of the invention, from known thiopeptide producing strains and using probes or primers derived of SEQ ID NO:35-46, are well known in the art. For example, genomic DNA of a thiopeptide producing strain can be amplified using primers flanking the region encoding the conserved region of each gene and determined from the disclosed nucleotide sequences.

Also included are modifications of said nucleic acids. Such modifications include, for example, labels which are known in the art, methylation, and substitution of one or more of the naturally occurring nucleotides with a degenerate nucleotide. These modifications may be useful to increase expression, yield, and/or to improve purification in the selected expression systems, or for another desired purpose.

In one embodiment, the nucleic acid of the invention is operatively linked to heterologous transcriptional and translational control sequence. More preferably, it is an expression vector suitable for expression in a host cell of whole or part of the core biosynthetic enzymes.

### 5. Production of Thiopeptide Precursor Protein and Corresponding Thiopeptides:

The determination of a chromosomal encoded backbone and consequently a ribosomal route for thiopeptide biosynthesis by the inventors permits, in one embodiment, one of ordinary skill in the art to clone and express the thiopeptide biosynthesis pathway, i.e the biosynthesis gene cluster, and thus, produce a thiopeptide compound, in a modified host cell or a heterologous organism. The invention also permits the production of thiopeptide precursors to be expressed in a heterologous host cell, i.e., another strain, than the natural producing strain. Although the examples illustrate use of a bacterial strain, any organism or expression system can be used, as described herein. The choice of organism is dependent upon the needs of the skilled artisan. For example, a strain that is amenable to genetic manipulation may be used in order to facilitate modification and production of thiopeptide compounds.

Thus, in another aspect, the invention relates to a host cell comprising one or more nucleic acids as described above encoding thiopeptide precursor protein and/or core biosynthetic enzymes, wherein said nucleic acid is not naturally found in the genome of said host cell and/or said host cell does not naturally produce corresponding thiopeptide.

Alternatively, the production of thiopeptide or thiopeptide derivatives is rendered possible by providing a culture medium comprising an appropriate amount of thiopeptide precursor protein as defined above, and culturing microorganisms in said culture medium, wherein said microorganism further comprises other genes required for thiopeptide biosynthesis, for example, the genes encoding the core biosynthetic enzymes.

As used herein, the term “host cell” or “microorganisms” means any cell of any organism that is selected, modified, transformed, grown or used or manipulated in any way for the production of thiopeptide precursor or thiopeptides and their derivatives by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The host cell or microorganism itself may be selected from any biological organism, including prokaryotic (e.g., bacterial) cells, plant cells, and eukaryotic cells, including, insect cells, yeast cells and mammalian cells. Representative examples of appropriate host cells include bacterial cells, such as, E. coli, Streptomyces and Bacillus subtilis cells; fungal cells, such as yeast cells, such as Pichia or Saccharomyces cells, filamentous fungi such Trichoderma or Aspergillus cells; and insect cells such as Drosophila S2 and Spodoptera Sf9 cells. Preferably, said host cells are selected from host cells known to synthesize thiopeptide derivatives or described to be resistant to said thiopeptide such as strains of Streptomyces ramocissimus and Streptomyces coelicolor (Olsthoorn-Tieleman et al. ‘Elongation factor Tu3 (EF-Tu3) from the kirromycin producer Streptomyces ramocissimus is resistant to three classes of EF-Tu-specific inhibitors.’ J. Bacteriol. 2007 May; 189(9):3581-90.). In some embodiments, the host cell is selected from a strain which is rendered resistant to thiopeptides. Methods to render resistant a strain are well known in the art [Kieser T, Bibb M J, Buttner M J, Chater K F, Hopwood D. Practical Streptomyces Genetics. John Innes Foundation, Norwich (2000)] and an example of such method is given in the examples below.

In some embodiments of the production method, said host cell further comprises other genes required for thiopeptide biosynthesis. Other genes required for biosynthesis can include for example one or more genes encoding the core biosynthetic enzymes as described above. Said host cells are for example selected among the group of species consisting of Nonomuraea sp., Planobispora sp., Amycolatopsis sp. and Streptomyces sp. Specific host cell organisms contemplated herein include, without limitation, organisms of the actinomycete suborder Streptosporangineae including the families Nocardiopsaceae, Streptosporangiaceae and Thermomonosporaceae, of which preferred genera include Acrocarpospora, Actinomadura, Herbidospora, Microbispora, Microtetraspora, Nocardiopsis, Nonomuraea ((Nonomuria sic, corrected by Chiba et al (1999) to Nonomuraea) a reclassified genus as reported by Zhenshui Zhang, Yue Wang and Jisheng Ruan in the International Journal of Systematic Bacteriology (1998), 48, 411-422)), Planobispora, Planomonospora, Planopolyspora, Planotetraspora or Streptosporangium. More generally, the terms are intended to encompass all organisms containing genetic information necessary to produce a thiopeptide compound. An example of such host cell includes Nonomuraea microbial strain Bp3714-39, a deposit of which was made on 2006-11-30, under Accession No. DSM 18831.

Suitable production techniques for protein such as the thiopeptide precursor protein of the invention are well known to those of skill in the art. See, e.g., Sambrook et al, Molecular Cloning: A Laboratory Manual, Cold Spring Harbor Press (Cold Spring Harbor, N.Y.). The sequences of any of the amino acid sequences provided herein can be readily generated using a variety of techniques. These and other suitable production methods are within the knowledge of those of skill in the art.

In one aspect, the amino acid sequences of the invention are produced by expression of one or more of the ORFs or genes in a selected host cell. The invention therefore relates to a method for producing a thiopeptide precursor protein, said method comprising the step of culturing a host cell capable of expressing a nucleic acid encoding a thiopeptide precursor protein as described above, and optionally, one or more nucleic acids encoding a core biosynthetic enzyme, under conditions appropriate for production of said thiopeptide precursor protein. In such method, said host cell is not the natural producing strain of said thiopeptide precursor protein, or when said host cell is the natural producing strain of said thiopeptide precursor protein, said nucleic acid encoding a thiopeptide precursor protein is a recombinant or heterologous nucleic acid. Depending on the host cell that is used, thiopeptide compound biosynthesis can be achieved by said host cell from the thiopeptide precursor. For that purpose, the person skilled in the art can either use a host cell that naturally synthesizes the enzymes required for post-translational modification of the thiopeptide precursor protein or introduce said genes required for post-translational modification for thiopeptide biosynthesis in the producing strain. In one specific embodiment, the methods defined above further comprise isolating said thiopeptide precursor or thiopeptide compound in a substantially pure form.

In a specific embodiment, the invention relates to the use of a host cell of the invention for the production of a thiopeptide compound selected among the group consisting of: GE2270A, GE37648A, Amythiamicin and the novel Ef-tu inhibitors as represented in formulas I to XI above, micrococcin, thiostrepton, nosiheptide, thiocillin, thiocins, nocathiacins, berninamycin, A10255B and radamycin.

In one aspect, the present invention provides methods of producing thiopeptide derivatives, comprising
i) synthesizing an altered thiopeptide precursor in a host cell by gene expression of the sequence encoding said altered thiopeptide precursor in said host cell,
ii) synthesizing said thiopeptide derivatives from said altered thiopeptide precursor; and/or,
iii) modifying said altered thiopeptide precursor by use of one or more of the core biosynthetic enzymes.

As used herein, an “altered thiopeptide precursor” is a thiopeptide precursor that is not naturally found in the strain producing it, i.e the producing strain.

Method for synthesizing altered thiopeptide precursor are further described below. Preferably, said altered thiopeptide derivative precursor is a variant of SEQ ID NO:1, SEQ ID NO:5 or SEQ ID NO:11 as described above. Step ii) is performed either in vitro, ie, outside of the host cells; or, in vivo, in the same host cell as step i). In the latter, one or more of the core biosynthetic enzymes are also synthesized by said host cells, either naturally or from recombinant DNA.

In a method of producing thiopeptide derivatives, the culture media inoculated with the host cell of the invention may be incubated under aerobic conditions using, for example, a rotary shaker or a stirred tank fermentor. Aeration may be achieved by the injection of air, oxygen or an appropriate gaseous mixture to the inoculated culture media during incubation. As soon as a sufficient amount of the thiopeptide derivatives have accumulated, they may be concentrated and isolated from the culture in conventional and usual manner, for example by extraction- and chromatographic methods, precipitation or crystallization, and/or in a manner disclosed herein. As an example for extraction, the culture can be mixed and stirred with a suitable organic solvent such as n-butanol, ethyl acetate, cyclohexane, n-hexane, toluene, n-butyl acetate or 4-methyl-2-pentanone, the thiopeptide derivatives in the organic layer can be recovered by removal of the solvent under reduced pressure. The resulting residue can optionally be reconstituted with for example water, ethanol, methanol or a mixture thereof, and re-extracted with a suitable organic solvent such as hexane, carbon tetrachloride, methylene chloride, dichloromethane or a mixture thereof. Following removal of the solvent, the compounds may be further purified for example by chromatographic methods. As an example for chromatography, stationary phases such as silica gel or aluminia oxide can be applied, with organic eluting solvents or mixtures thereof, including ethers, ketones, esters, halogenated hydrocarbons or alcohols, or reversed-phase chromatography on modified silica gel having various functional groups and eluting with organic solvents or aqueous mixtures thereof, like acetonitrile, methanol or tetrahydrofuran at different pH. Another example is partition-chromatography, for example in the solid-liquid or in the liquid-liquid mode. Also size exclusion chromatography may be applied, for example using Sephadex LH-20 (Sigma-Aldrich) and eluting with different solvents, preferably with alcohols.

As it is usual in this field, the production as well as the recovery and purification process may be monitored by a variety of analytical methods, including bioassays, TLC, HPLC or a combination thereof, and applying different detection methods, for TLC typically UV light, iodine vapor or spraying coloring reagents, for HPLC typically UV light, mass sensitive or light scattering methods. For example a HPLC technique is represented by using a reversed-phase column with a functionalized silica gel and applying an eluent which is a linear gradient mixture of a polar water miscible solvent and water at a specific pH, and a detection method with UV light at different wavelengths and a mass sensitive detector.

The resulting purified compounds are free of cells and cellular materials, by-products, reagents, and other foreign material as necessary to permit handling and formulating of the compound for laboratory and/or clinical purposes. It is preferable that purity of the compounds used in the present invention have a purity of greater than 80% by weight; more preferably at least 90% by weight, even more preferably greater than 95% by weight; yet even more preferably at least 99% by weight. In one embodiment, the invention provides compositions containing the compounds of the invention, regardless of how such compounds are produced.

The compounds biosynthesized by the host cells may optionally be subjected to random and/or directed chemical modifications to form compounds that are derivatives or structural analogs. The compounds may optionally be modified using methods known in the art and described herein.

### 6. Mutant Microorganism Capable of Producing Thiopeptide Precursor Protein for Thiopeptide Derivatives Production

Following the teaching of the present invention, it is now possible to genetically manipulate microorganism capable of producing thiopeptide precursor protein, for example with the aim of improving thiopeptide production or modulating thiopeptide structure. Accordingly, in a further aspect, the invention provides a mutant microorganism, wherein said mutant microorganism has a mutation in the gene encoding the thiopeptide precursor protein and/or in one or more of the genes encoding core biosynthetic enzymes. Said mutation can be a single or multiple nucleotide deletion, insertion or substitution. It can also be a deletion of a fragment of the gene or the whole gene encoding the thiopeptide precursor protein. Preferably, the mutant microorganism no longer expresses the gene encoding the thiopeptide precursor protein of the invention when compared to the corresponding wild-type microorganism. Preferably, to avoid polar affects said mutation is an in-frame deletion within the gene encoding the thiopeptide precursor protein.

Said mutant organisms is for example of the actinomycete suborder Streptosporangineae including the families Nocardiopsaceae, Streptosporangiaceae and Thermomonosporaceae, of which preferred genera include Acrocarpospora, Actinomadura, Herbidospora, Microbispora, Microtetraspora, Nocardiopsis, Nonomuraea ((Nonomuria sic, corrected by Chiba et al (1999) to Nonomuraea) a reclassified genus as reported by Zhenshui Zhang, Yue Wang and Jisheng Ruan in the International Journal of Systematic Bacteriology (1998), 48, 411-422)), Planobispora, Planomonospora, Planopolyspora, Planotetraspora or Streptosporangium.

In a specific embodiment, said mutant microorganism is a Nonomuraea sp., for example, Nonomuraea microbial strain Bp3714-39, a deposit of which was made on 2006-11-30, under Accession No. DSM 18831, and said mutation is a disruption of the gene comprising SEQ ID NO:15 or SEQ ID NO:16, for example a mutation of the gene comprising SEQ ID NO:17 or SEQ ID NO:18.

In another particular embodiment, the mutant microorganism of the present invention can be further transformed with a nucleic acid encoding any of the thiopeptide precursor proteins as described above. This method allows to provide with microorganisms capable of thiopeptide biosynthesis from any thiopeptide precursor protein, including the variants of SEQ ID NO:1, SEQ ID NO5 or SEQ ID NO:11 as described above. In one further embodiment, said mutant microorganism further expresses either naturally or recombinantly at least one or more genes encoding the core biosynthetic enzymes, for example one or more genes encoding a polypeptide selected among SEQ ID NO:23-34. In one embodiment, the mutant microorganism expresses either naturally or recombinantly the genes encoding at least the polypeptides of SEQ ID NOs:23-28. In another embodiment, the mutant microorganism expresses either naturally or recombinantly the genes encoding at least the polypeptides of SEQ ID NOs:35-46.

### 7. Screening of Novel Strains Producing Thiopeptide Compounds

The genes identified by the inventors can further be used as a tool to identify other microorganisms capable of producing thiopeptide derivatives. For example, the invention relates to any method that allows one to identify cells having a gene substantially similar to either,

(i) the gene of SEQ ID NO:17 or SEQ ID NO:18 and more preferably the fragment of SEQ ID NO:15 or SEQ ID NO:16, or
(ii) the core genes as defined in any of SEQ ID NOs:35-46.

In a specific embodiment, two DNA sequences are “substantially homologous” or “substantially similar” when at least about 80%, and most preferably at least about 90% or 95% of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length, more preferably at least about 14 amino acids, and may be up to about 700 amino acids. Examples of suitable fragments are described herein.

In one embodiment, the method to identify cells capable of producing thiopeptide derivatives, comprises the following steps of

(i) incubating genomic DNA or RNA from isolated cells with a nucleic acid probe of SEQ ID NO:15 or SEQ ID NO:16 or specific fragments thereof for specific hybridization of the probe to homologous DNA region; and,
(ii) identifying the cells which comprise genomic DNA region or RNA that hybridizes specifically with said probe according to step (i).

A nucleic acid molecule is “hybridizable specifically” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., Molecular Cloning: A Laboratory Manual, Second Edition (1989) Cold Spring Harbor Laboratory Press, Cold Spring Harbor, N.Y. (herein “Sambrook et al., 1989”). The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SCC. SCC is a 0.15M NaCl, 0.015M Na citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, standard hybridization conditions are used. The term “standard hybridization conditions” refers to a Tm of 55° C., and utilizes conditions as set forth above. In a preferred embodiment, the Tm is 60° C.; in a more preferred embodiment, the Tm is 65° C. In a specific embodiment, “high stringency” conditions are used.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligonucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

In one preferred embodiment of the method, oligonucleotide specific of the gene encoding thiopeptide precursor are designed and used for identification of novel cells capable of producing thiopeptide compounds. Such oligonucleotides can be used for PCR amplification of a fragment of the gene encoding thiopeptide precursor protein. Preferably, lower eukaryotic cells are screened, and more preferably cells from actinomycetes species.

### 8. Genetic Manipulation of Ilene Encoding Thiopeptide Precursor Protein and Use for Screening Novel Thiopeptide Derivatives

In another embodiment, the present invention provides methods of modifying one or more of the nucleotide sequence of the genes and/or open reading frames encoding thiopeptide precursor protein. For example, such modifications or alterations can be for the purpose of improving expression in a selected expression system or generating novel thiopeptide derivatives. Other alterations can be made to extinguish, modify, or enhance function of thiopeptide compounds, including improving antibiotic function or decreasing undesired properties.

In one aspect, synthesis of an altered thiopeptide precursor is achieved from modification of the nucleic acid sequence encoding thiopeptide precursor protein. In one embodiment, the altered nucleic acid sequences can be provided to a heterologous host cell as described above via a suitable vector in a selected host cell and used to express the corresponding product. Alternatively, the alteration can be made directly in the natural gene carried by strain producing thiopeptide, for example, by genetic manipulation of said strain.

The present invention contemplates any method of altering any of the nucleic acid sequences encoding the precursor proteins of the present invention. More specifically, the invention contemplates any method that inserts amino acids, deletes amino acids or replaces amino acids in the proteins of the invention. The modifications may be performed at the nucleic acid level. These modifications are performed by standard techniques and are well known within the art.

Accordingly, the present invention provides a method to generate a nucleic acid library encoding precursor of thiopeptide derivatives, said method comprising the steps of generating a variety of nucleic acid with altered nucleotide sequence by performing nucleotide substitution in at least one codon of the sequence encoding of the sequence encoding any of SEQ ID NO:1-14, for each nucleic acid. Such nucleic acid library can be used advantageously to screen for novel thiopeptide derivatives, for example, Ef-tu inhibitor, with improved properties.

The altered nucleic acids or nucleic acid library can then be used to transform host cells for thiopeptide production as described above.

Preferably, each nucleic acid of the nucleic acid library has a single nucleotide substitution so that only one codon of the sequence encoding any of SEQ ID NO:1-14 is mutated compared to wild type corresponding sequence. Method to generate site-directed mutagenesis or nucleic acid libraries are well known in the art and for example described in an article by Hogrefe et al published in Biotechniques (‘Creating randomized amino acid libraries with the QuikChange Multi Site-Directed Mutagenesis Kit.’ 2002 November; 33(5):1158-60, 1162, 1164-5).

In one specific embodiment, said nucleotide substitution is performed in one or more of the codons encoding amino acid residues of positions 2, 5, 7, 8, 14 of any of SEQ ID NO:1-4.

Preferably the host cells are capable of synthesizing thiopeptide compounds, ie, further comprising other genes required for thiopeptide biosynthesis. For example, other genes required for thiopeptide biosynthesis may comprise one or more genes encoding a polypeptide selected among SEQ ID NO:23-34. More preferably, the altered nucleic acids are transformed with an expression vector, so that, corresponding thiopeptide precursor protein is synthesized after transformation from said expression vector. The resulting expression library can be used as a tool to screen novel thiopeptide derivatives, for example, novel Ef-tu inhibitors.

### 9. Cloning of Tailoring Biosynthetic Genes

When characterizing the biosynthetic gene clusters from two strains producing distinct thiopeptide compounds, the inventors identified ORFs that are likely involved in thiopeptide biosynthesis but that are strain-specific. It is therefore proposed that these genes likely encode tailoring polypeptides, mainly enzymes and transcriptional regulators, involved in gene regulation and enzymatic modification of the core thiopeptide structure to produce the final strain-specific thiopeptide compounds.

The Tables 3 and 4 describes the nucleic acid and corresponding polypeptide sequences of these tailoring polypeptides, according to the invention.

TABLE 3
Examples of tailoring polypeptides for thiopeptide biosynthesis from Strain I

| | Polypeptide | Coordinates of | Homology and |
|---|---|---|---|
| ORF# | SEQ ID | genomic BAC | Proposed function |
| ORF1-I | NO: 47 | 1(GTG)-714(TGA) | Transcriptional regulator/ |
| | | of NO: 62 | Regulation gene expression |
| ORF2-I | NO: 48 | 711(GTG)-2612(TGA) | Adenylation - |
| | | of NO: 62 | heterocyclization/ |
| | | | Oxazoline formation in |
| | | | Tail |
| ORF3-I | NO: 49 | 2609(GTG)-5098(TGA) | Lantibiotic dehydratase/ |
| | | of NO: 62 | Serine −> Dehydroalanine |
| ORF4-I | NO: 50 | 5095(GTG)-6129(TAG) | Cytochrome P450 |
| | | of NO: 62 | monooxygenase/ |
| | | | Hydroxylation |
| ORF5-I | NO: 51 | 6218(ATG)-6904(TGA) | ? |
| | | of NO: 62 | |
| ORF6-I | NO: 52 | 6947(ATG)-8065(TGA) | N-Methylase/Methylation |
| | | of NO: 62 | of asparagine |
| ORF7-I | NO: 21 | 8094(ATG)-9338(TGA) | Coproporphyrinogen III |
| | | of NO: 62 | oxidase/Methylation of |
| | | | thiazole |
| ORF15-I | NO: 53 | 23718(ATG)-25454(TGA) | ? |
| | | of NO: 62 | |
| ORF16-I | NO: 54 | 19327(ATG)-20577(TGA) | Coproporphyrinogen III |
| | | of NO: 62 | oxidase/Methlyation |
| | | | (methoxy formation) |
| ORF17-I | NO: 55 | 20579(GTG)-21598(TGA) | O-methylase/Methlyation |
| | | of NO: 62 | (methoxy formation) |
| ORF18-I | NO: 56 | 22234(ATG)-22848(TAG) | Deaminase Reductase/ |
| | | of NO: 62 | Removal N-terminus - |
| | | | Pyridine |

TABLE 4
Examples of tailoring polypeptides for thiopeptide biosynthesis from Strain II

| | Polypeptide | Coordinates of | Homology and |
|---|---|---|---|
| ORF# | SEQ ID | genomic BAC | Proposed function |
| ORF1-II | NO: 47 | 1(TAG)←1213(ATG) | Coproporphyrinogen III |
| | | of NO: 63 | oxidase/Methylation of |
| | | | thiazole |
| ORF2-II | NO: 48 | 1209(TGA)←2373(ATG) | Cytochrome P450 |
| | | of NO: 63 | monooxygenase/ |
| | | | hydroxylation |
| ORF3-II | NO: 49 | 2369(TGA)←3590(GTG) | Cytochrome P450 |
| | | of NO: 63 | monooxygenase/ |
| | | | hydroxylation |
| ORF4-II | NO: 50 | 3600(TGA)←4332(ATG) | Transcriptional regulator/ |
| | | of NO: 63 | regulation gene |
| | | | expression |
| ORF12-II | NO: 51 | 14252(ATG→15587(TGA) | Peptidase/ |
| | | of NO: 63 | Amidohydrolase/ |
| | | | removal C-terminal |
| | | | alanine |

In one embodiment, one or more of the polypeptides listed in Table 3 or 4 are used for in vitro or in vivo synthesis of a compound of formula (I) to (XI) as defined above.

The invention relates also to any functional variant of such enzymes reported in Tables 3 or 4, retaining substantially the same enzymatic activity. The invention also relates to any functional variant of any transcriptional regulator reported in Tables 3 or 4, retaining substantially the same transcriptional activity. In one embodiment, such polypeptide contains not least than 1, 2, 3, 4 or 5 amino acids deleted, inserted or substituted when compared to the original polypeptide listed in the table above. In another embodiment, such functional variants are at least 80 or 90% identical to one of the polypeptide listed above.

These sequences can be used to enable the generation of mutant strains that lack specific tailoring steps, for example, to avoid the production of undesired side compounds. In one embodiment, the invention relates to a mutant strain capable of producing thiopeptide that is deficient in the expression of one or more of the tailoring genes listed in the table above. In one specific embodiment, the mutant strain is deficient in the expression of a gene encoded by ORF2-II or ORF3-II (respectively SEQ ID NO:58 and SEQ ID NO:59) or ORF4-I (SEQ ID NO:50).

As used herein, “deficient expression” means that the corresponding polypeptide is no longer expressed by the mutant strain when compared to the wild type strain, or at least has steady state mRNA amounts decreased by more than 50% or more than 90%, compared to wild type strain steady-state mRNA as measured in conventional methods for quantifying mRNA expression. For example, the gene is disrupted or partially or fully deleted so that a functional protein is no longer synthesized.

These sequences can further be used in combination with the gene encoding precursor protein and genes encoding core enzymes to engineer host cells capable of producing specific thiopeptide derivatives. In one embodiment, the invention relates to a host cell that contains recombinant open reading frames ORF1-II to ORF12-II, as described in Tables 2, 3 and 4, or functional variants thereof having at least 80%, or at least 90% identity to the corresponding wild type sequence, and capable of producing an EF-Tu inhibitor. In another embodiment, the invention relates to a host cell that contains recombinant ORF1-I to ORF18-I as described in Tables 2, 3 and 4 or functional variants thereof having at least 80%, or at least 90% identity to the corresponding wild type sequence, and capable of producing an EF-Tu inhibitor.

In another embodiment, the isolated tailoring enzymes can be used either individually or in combination as a catalyst in a chemical reaction step in an in vitro method, for example using a thiopeptide precursor protein as a starting material, for example, in a method for producing a thiopeptide compound, for example, in a method for producing an E-FTu inhibitor.

### BRIEF DESCRIPTION OF THE FIGURES

FIG. 1. Thiopeptide structural gene. Potential initiation sites in bold. The 14 amino-acid backbone is underlined.

FIG. 2. Biosynthetic gene cluster from thiopeptide producing Strain I. Arrows represent hypothetical promoters. Open arrows represent open reading frames: the black filled arrow indicates the seriesI structural gene and grey shaded arrows are syntenous genes shared by both the seriesI and seriesII thiopeptide gene clusters. HindIII and EcoRI are unique restriction sites flanking the cluster.

FIG. 3 Biosynthetic gene cluster from thiopeptide producing Strain II (strain Bp3714-39). Arrows represent hypothetical divergent promoters. Open arrows represent open reading frames: The black filled arrow indicates the seriesII structural gene and grey shaded arrows are syntenous genes shared by both the seriesI and seriesII thiopeptide gene clusters. The gene cluster is flanked by PstI restriction sites.

### EXAMPLES

The invention is also described by means of particular examples. However, the use of such examples is illustrative only and in no way limits the scope and meaning of the invention or of any exemplified term.

1. Identification of a Small Structural Gene in the Genome of the Thiopeptide Producing Nonomuraea sp., which Encoded the Whole Peptide Backbone

A PCR method was employed to isolate a chromosomal sequence that encodes for the thiopeptide backbone. Genomic DNA was purified from the thiopeptide producing Nonomuraea strain originating from Hubei province, China and digested with the restriction enzyme Nail. The digested chromosomal DNA was purified by passing it over a QiaQuick DNA clean-up column (Qiagen). An adaptor-ligated library was generated by ligating to the fragmented DNA a 500-fold molar excess of the following adapter: 5′-CGACCACGACCA (phosphorylated on the 5′ terminus and includes a 3′ C6-TFA-amino modification) and 5′-AGTCTCGCAGATGATAAGGTGGTCGTGGT. The thiopeptide structural gene was amplified from this library by using the adapter primer (5′-GTCCAGTCTCGCAGATGATAAGG) and a degenerate primer designed on the thiopeptide macrocycle (CFGCVCNC: 5′-CARAAICCRCAIACRCARTTRCA). Inosine was included in the oligonucleotide to reduce the degeneracy. HotStar polymerase mix (Qiagen) was used to obtain a specific PCR product with the cycling conditions as follows: 95° C. for 15 min; 30 cycles of 94° C. for 30 sec, 55° C. for 30 sec, and 72° C. for 1 min; and 72° C. for 10 min. The 3′ amino-modification on the adaptor blocks extension and prevents adaptor-adaptor amplification by the adaptor primer. Amplification can only take place if the degenerate oligonucleotide anneals and primes a polymerase extension that generates the complementary sequence for the adaptor primer.

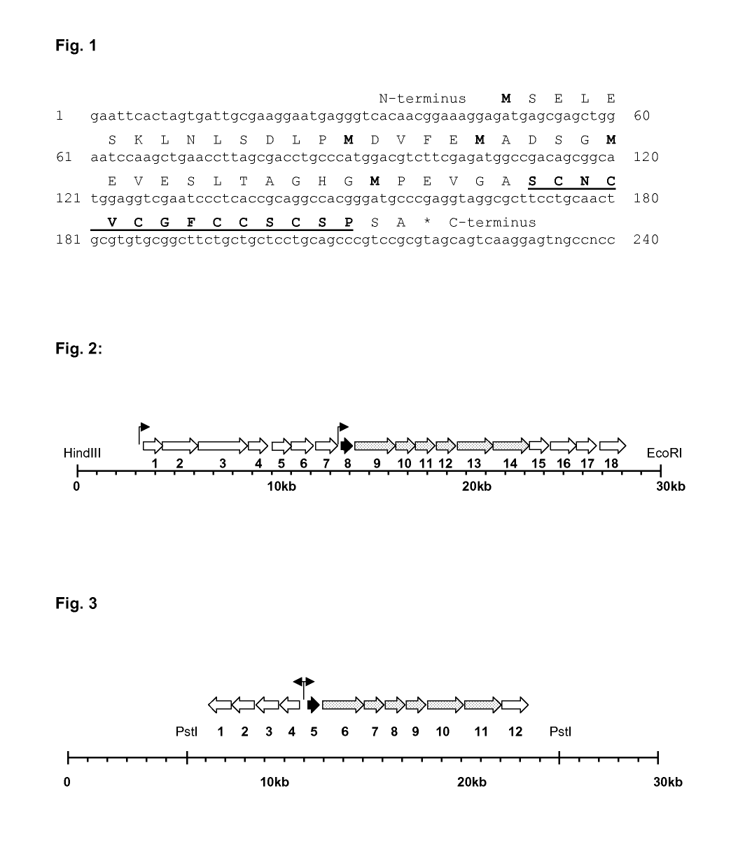

Experiments were successful in identifying a small structural gene in the genome of the thiopeptide producing Nonomuraea sp., which encoded the whole peptide backbone (FIG. 1). The predicted size of this precursor protein is 57-amino-acids. However, there are a number of initiation codons that may indicate alternative translation start sites. The 14 amino-acid thiopeptide sequence is at the C-terminus and establishes the direction of synthesis. The primary amino acid sequence of the thiopeptide backbone begins with a serine that is incorporated into the pyridine ring and continues in an anticlockwise direction around the macrocycle and finishes with the final amino-acid at the end of the side-chain. Homology searches in the public databases revealed no close homolog.

### 2. Genetic Disruption of the Gene Encoding Thiopeptide Precursor Protein

The suicide vector pSET152-Hind can replicate in E. coli, confers apramycin resistance and carries an origin of transfer (oriT) that allows intergeneric conjugation from E. coli into actinomycete species. pSET152-Hind is a derivative of the broad host range vector pSET152. The gene that allows site-specific intergration (int) was deleted from pSET152 by removing a HindIII fragment. The gene encoding the thiopeptide precursor protein may be disrupted in either of two ways; insertional inactivation or deletion. The first method requires that an internal fragment of the gene without an initiation or stop codon be cloned into pSET152-Hind. This plasmid may then be introduced into the thiopeptide producing strain by conjugation. Selection for apramycin will identify mutants that have the vector backbone inserted into the thiopeptide structural gene. Such an event will inhibit transcription and translation and prevent the production of thiopeptide. The second method requires a mutant allele be constructed in pSET152-HindIII and then transferred into the producing strain. The mutant allele will contain the sequence upstream and downstream the structural gene but will have the open reading frame deleted preferably in-frame. This deletion may be marked/replaced with a gene that confers resistance to an antibiotic such as hygromycin or thiostrepton. On conjugation into the producing strain selection for the plasmid antibiotic marker will select for strains that have both the wild type and mutant allele in the chromosome. Merodiploidy results from homologous recombination between the plasmid and the chromosome in either the upstream or downstream sequence. Selection for the loss of the vector antibiotic marker and subsequent PCR screening for deletion or antibiotic selection for the marked mutant allele will identify the desired second recombination event, which will remove the wild type allele and leave the deleted allele of the structural gene.

Alternatively, advances in DNA synthesis technology now allow the synthetic build-up of large pieces of DNA and such services are commercially available. The thiopeptide gene cluster maybe reengineered by de novo chemical synthesis for heterologous expression and thiopeptide production in a surrogate host. Such a host will have well established genetic tools eg. Streptomyces coelicolor or Streptomyces lividans and must be thiopeptide resistant or be rendered thiopeptide resistant. Resistant strains are isolated by plating 10¹⁰ to 10¹¹ cells/spores on agar plates containing concentrations of thiopeptide in excess of the thiopeptide minimum inhibitory concentration. Rare spontaneous mutants pre-existing in the population conferring resistance will be identified by colonial growth on the selection plates. The frequency of mutation rate may be increased by exposing cells to chemical mutagen. Chemical synthesis of the thiopeptide gene cluster permits the introduction of better regulatory elements, deletion of genes, removal or introduction of restriction sites and alterations in codon usage. A functional synthetic copy of the gene cluster cloned on the integrative shuttle vector pOJ436 or episomal shuttle vector pOJ446 will have restriction sites introduced into the structural gene to permit the generation of an in-frame deletion.

In addition, a cloned copy of the thiopeptide gene cluster or synthetic version may be precisely manipulated by homologous recombination in strains of E. coli which express phage-derived protein pairs, either RecE/RecT from the Rac prophage, or Reda/Redβ from λ phage. This technique is referred to as Red/ET Recombineering or λ-mediated recombination (Muyrers, J. P. P., Zhang, Y., Stewart, A. F. ET cloning: Think recombination first. Genetic Engineering, Principles and Methods (Ed. J. K. Setlow), 22, 77-98 Kluwer Academic/Plenum Publishers, NY. (2000)).

### 3. Heterologous Expression of the Structural Genes to Generate Alternative Thiopeptide Structures.

A native producing strain or heterologous host expressing the thiopeptide core and tailoring biosynthetic genes but with an in-frame deletion in the precursor structural gene are useful tool strains. These tool strains may be used to produce thiopeptides with alternative structures

Site-directed mutagenesis of the structural gene may be employed to replace or introduce new amino acids into the thiopeptide. The mutated version of the structural gene cloned in pHM11a or pSET152 is reintroduced by conjugation or transformation into the tool strain expressing the thiopeptide biosynthetic enzymes.

Alternatively, a library can be created encoding alternative amino acids in every position of the thiopeptide backbone. This variant library can be generated chemically by gene synthesis or by degenerate PCR. The PCR method requires amplification of a thiopeptide structural gene to incorporate variations in the thiopeptide encoding backbone and restriction sites for cloning and expression in a plasmid such as pHM11a or pSET152. One of the PCR primers will be degenerate to incorporate all amino acid substitutions. The degree of degeneracy can altered to allow substitutions in selected positions of the backbone ie. not in positions encoding amino acids deemed invariant such as the cysteines that form the thiazoles of the thiopeptide macrocycle. Both primers will be tagged with restriction sites to allow direct cloning of the PCR product into the expression vector. All E. coli transformants will be pooled and DNA isolated to generate the variant library. The library will be transformed into the tool strain and a bioassay may be employed to identify clones harboring structural genes that support production of active thiopeptides with alternative structures.

### 4. Isolation of the Gene Encoding Thiostrepton Backbone Polypeptide

A PCR method was employed to isolate a chromosomal sequence that encodes for the thiostrepton backbone. Genomic DNA was purified from the thiostrepton producing strain Streptomyces azureus ETH28555 and digested with the restriction enzyme NarI. The digested chromosomal DNA was purified by passing it over a QiaQuick DNA clean-up column (Qiagen). An adaptor-ligated library was generated by ligating to the fragmented DNA a 500-fold molar excess of the following adapter: 5′-CGACCACGACCA (phosphorylated on the 5′ terminus and includes a 3′ C6-TFA-amino modification) and 5′-AGTCTCGCAGATGATAAGGTGGTCGTGGT. The thiostrepton structural gene was amplified from this library by using the adapter primer (5-GTCCAGTCTCGCAGATGATAAGG) and a degenerate primer designed on the thiostrepton macrocycle (CTTCICTC: 5′-CAC GTG CAG ATR CAN GTN GTR CA-3′). The degenerate primer was designed based on the CODE HOP principles with a 5′ consensus clamp and a 3′ degenerate core (Rose et al. CODEHOP (COnsensus-DEgenerate Hybrid Oligonucleotide Primer) PCR primer design. Nucleic Acids Res. 2003 Jul. 1; 31(13):3763-6). HotStar polymerase mix (Qiagen) was used to obtain a specific PCR product with the cycling conditions as follows: 95° C. for 15 min; 40 cycles of 94° C. for 30 sec, 55° C. for 30 sec, and 72° C. for 1 min; and 72° C. for 10 min. The 3′ amino-modification on the adaptor blocks extension and prevents adaptor-adaptor amplification by the adaptor primer. Amplification can only take place if the degenerate oligonucleotide anneals and primes a polymerase extension that generates the complementary sequence for the adaptor primer. This strategy proved successful in identifying a genomic fragment that encoded the thiostrepton macrocycle. Gene specific primers were then used to walk both up and downstream to identify the full thiostrepton structural gene.

5. Biosynthetic Gene Clusters from Thiopeptide Producing Strains I and II

### 5.1 Strain I

FIG. 2 describes the ORFs positions in one isolated BAC (SEQ ID NO:62) from Strain I genomic DNA comprising the biosynthetic gene cluster for thiopeptide synthesis.

Without being bound by any preferred model, the following pathway defines putative function of the individual polypeptides characterized from the cloned biosynthetic gene clusters in the synthesis of thiopeptide derivatives.

### Synthesis Scheme for SeriesI:

(A) ORF9, ORF10, ORF11, ORF12, ORF13 and ORF 14 encode the core biosynthetic enzymes that likely forms a complex, which functions processively by binding the precursor peptide. The corresponding modifications are introduced as the complex moves along the peptide. Thiazoles are introduced by cyclodehydration of the cysteine thiol group with the preceding carbonyl and subsequent oxidation of thiazoline ring. Dehydration of two serine residues form the dehydroalanine residues that are the substrates for the aza-Diels-Alder cycloaddition reaction that yields the central pyridine heterocycle.

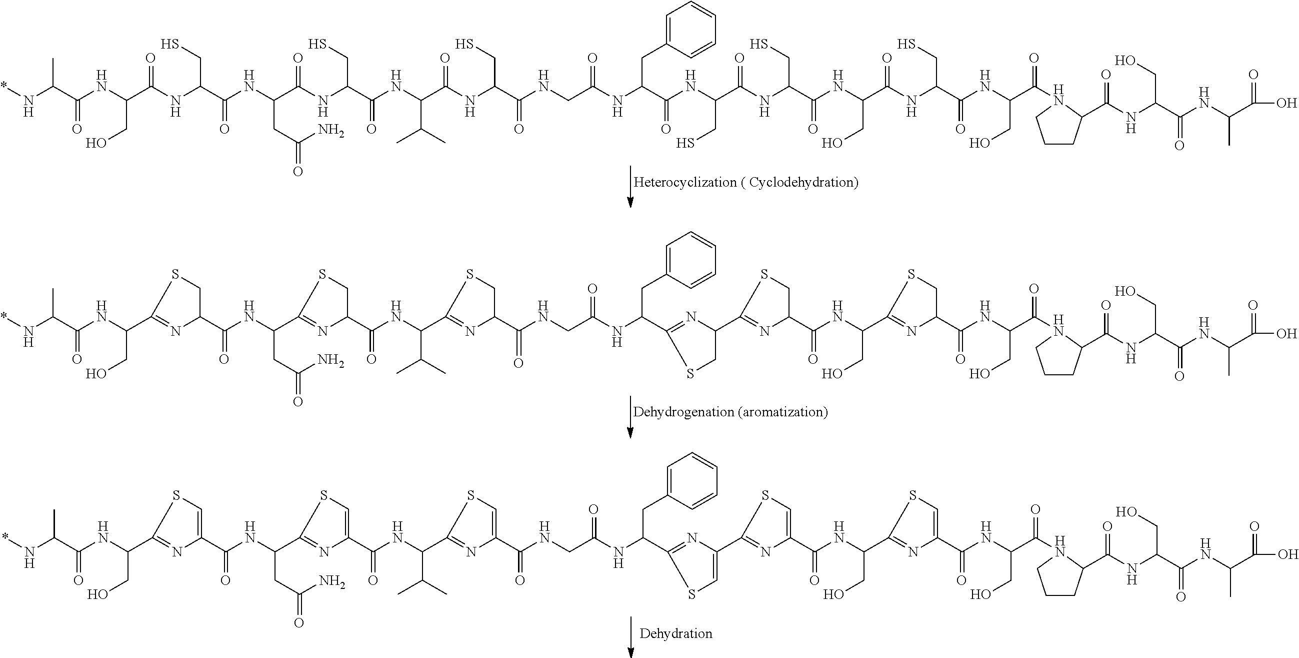

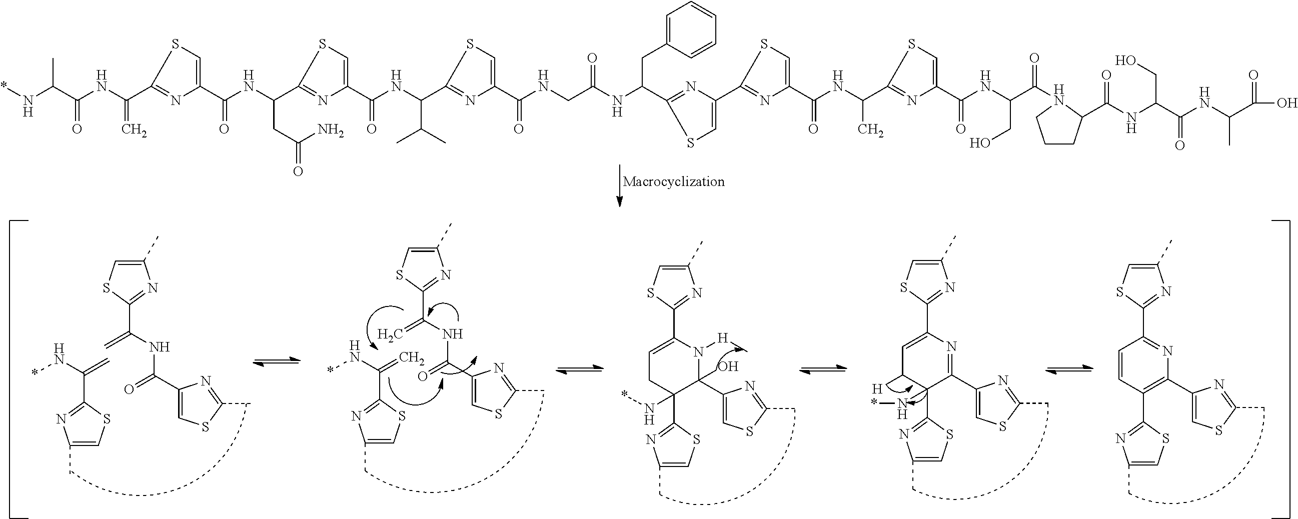

(B) ORF2 and ORF3 encode enzymes likely involved in the modifications incorporated into the tail. Cyclodehydration of a serine residue yields an oxazoline ring. Dehydration of the additional serine in the tail to dehydroalanine may require a separate step due to the presence of a proline in the tail, which likely causes a conformational kink in the peptide. ORF18 may be involved in elimination of terminal sequence leaving amide group.

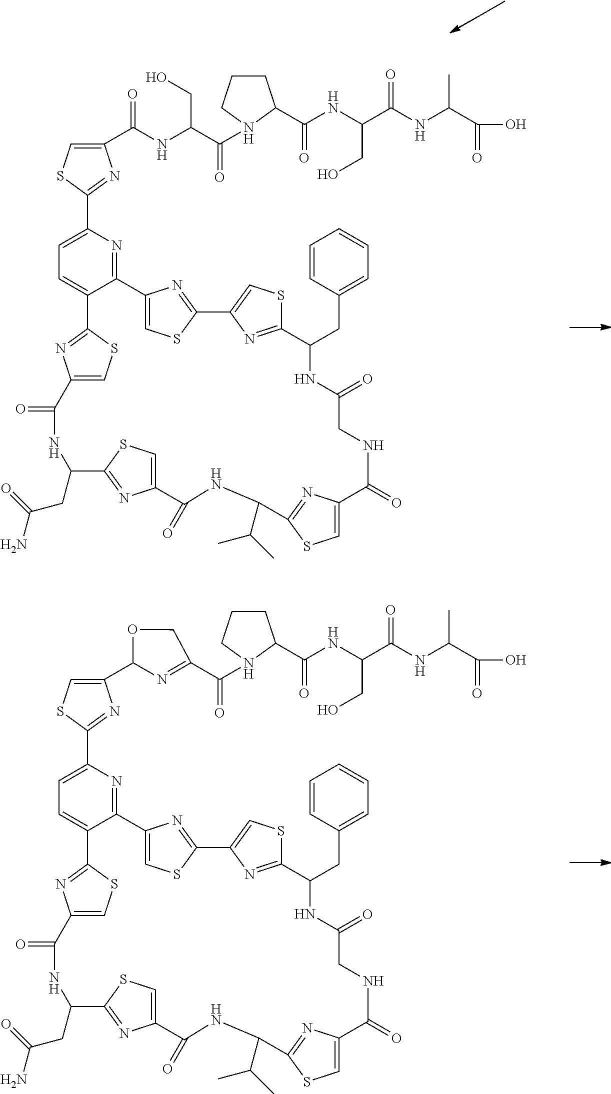

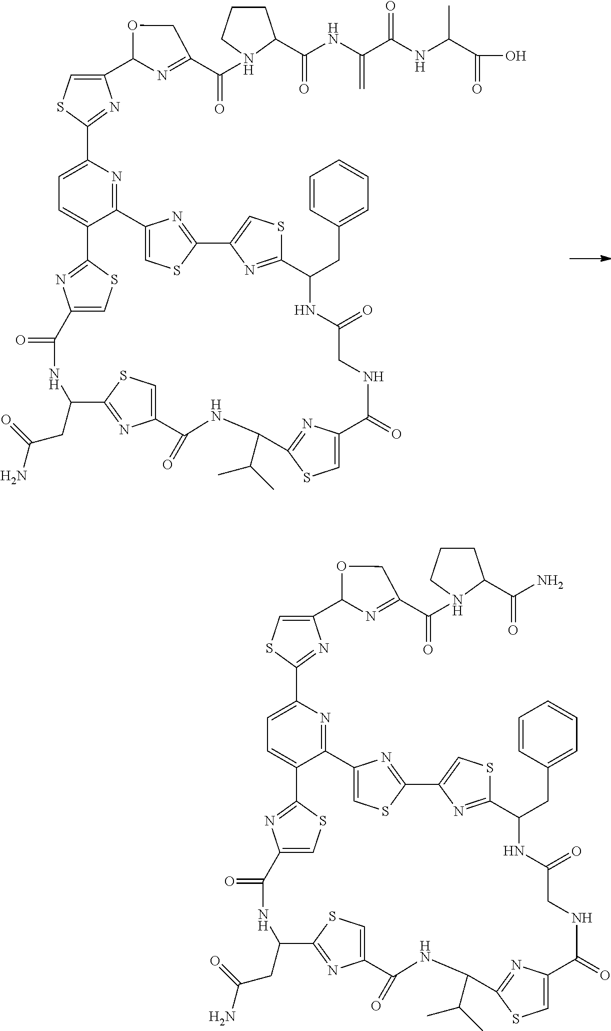

(C) ORF4, ORF6, ORF7, ORF16 and ORF17 encode enzymes likely involved in tailoring modifications. ORF4: hydroxylation of phenylalanine. ORF5: methylation of asparagine. ORF7: methylation of thiazole. ORF16 and ORF17: addition of methoxyethyl group to thiazole.

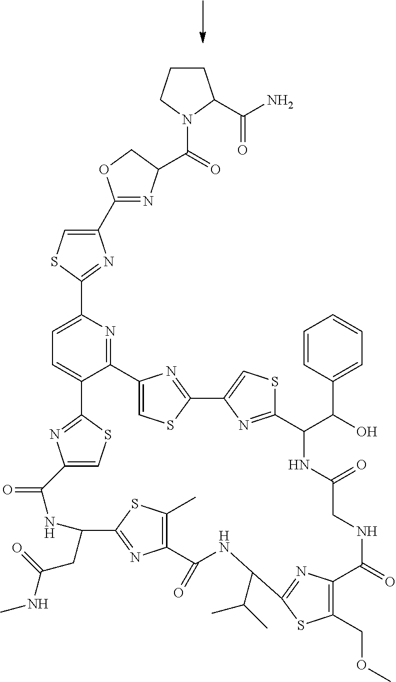

### 5.2 Strain II

FIG. 3 describes the ORFs positions in one isolated BAC (SEQ ID NO:63) from Strain II genomic DNA comprising the biosynthetic gene cluster for thiopeptide synthesis.

Without being bound by any preferred model, the following pathway defines putative function of the individual polypeptides characterized from the cloned biosynthetic gene clusters in the synthesis of thiopeptide derivatives.

### Synthesis Scheme for SeriesII:

(A) ORF6, ORF7, ORF8, ORF9, ORF10 and ORF 11 encode the core biosynthetic enzymes that likely forms a complex, which functions processively by binding the precursor peptide. The corresponding modifications are introduced as the complex moves along the peptide. Thiazoles are introduced by cyclodehydration of the cysteine thiol group with the preceding carbonyl and subsequent oxidation of thiazoline ring. Dehydration of two serine residues form the dehydroalanine residues that are the substrates for the aza-Diels-Alder cycloaddition reaction that yields the central pyridine heterocycle.

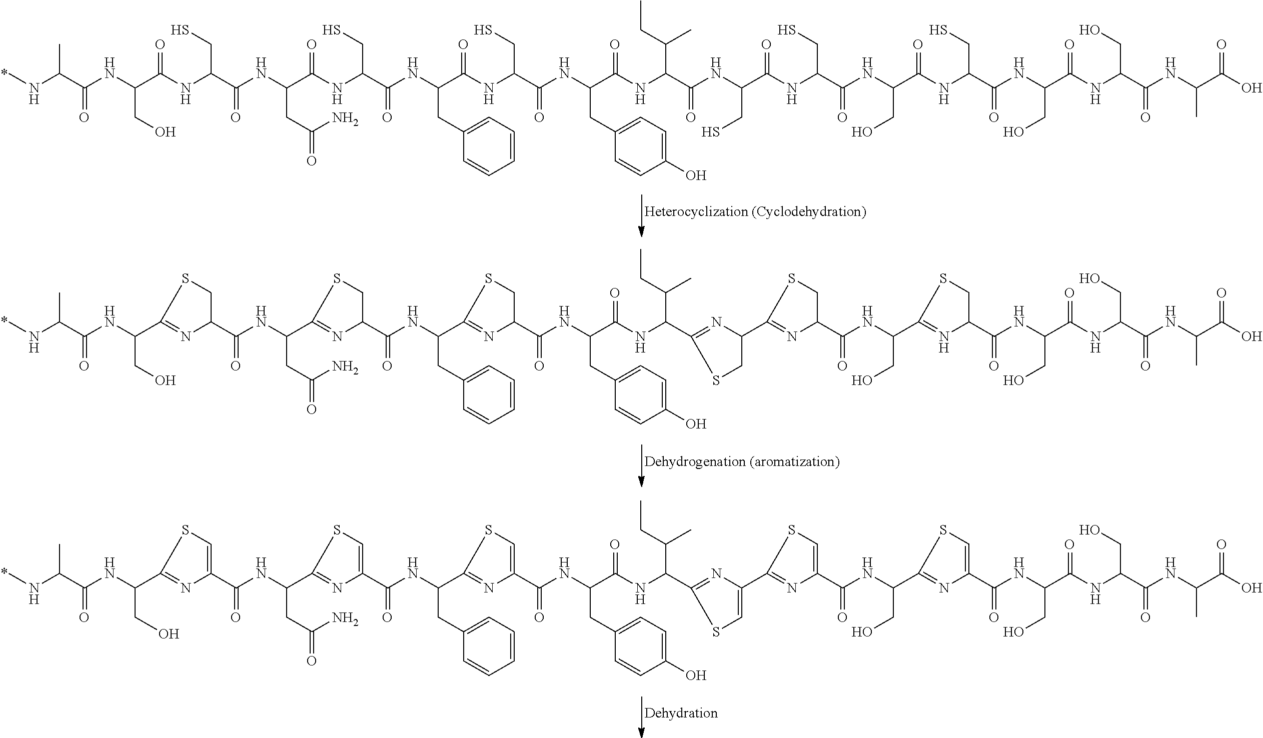

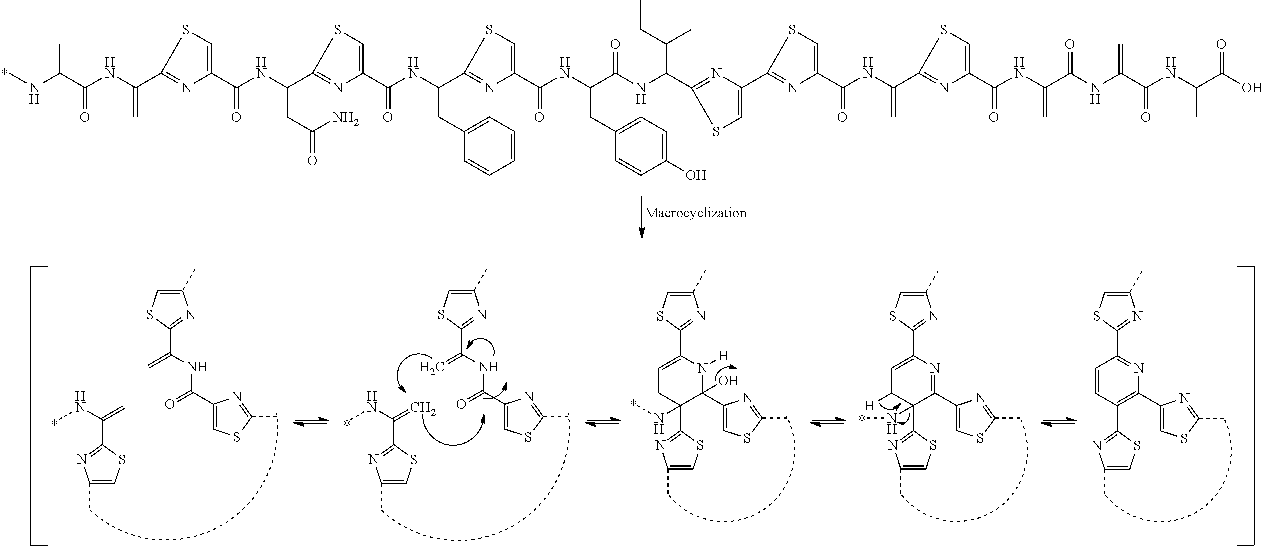

(B) ORF 12 encodes a peptidase that cleaves the terminal alanine

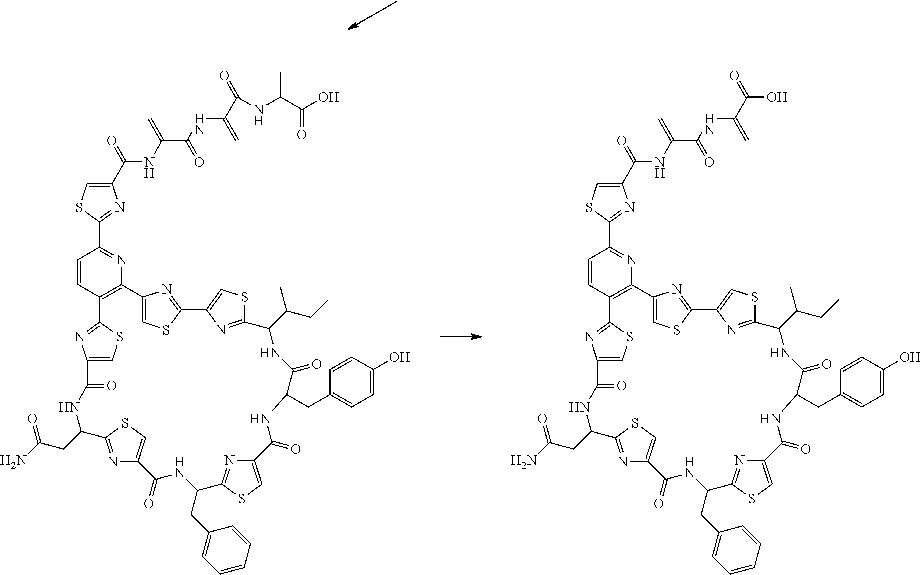

(C) ORF1, ORF2 and ORF3 encode enzymes likely involved in tailoring modifications. ORF1 methylation of thiazole with ORF2 and ORF3 involved in the hydroxylation of phenylalanine and isoleucine. The isoleucine is hydroxylated twice and decomposes to generate an epoxide.

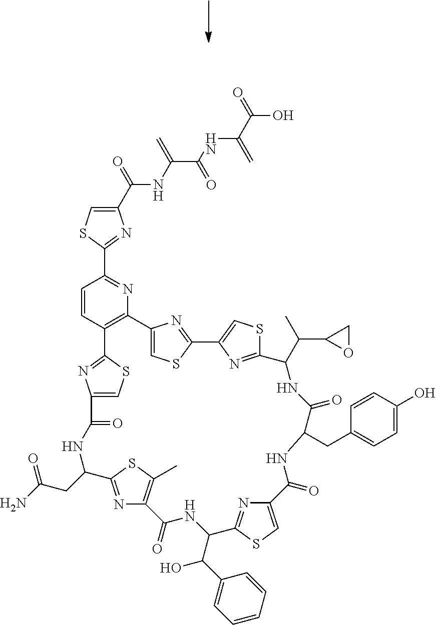

### 6. Production of Thiopeptide Derivatives

### Media Composition

(a) Seed Medium

| | Concentration |
|---|---|
| Substance | [g/l] |
| Agar | 1 |
| Trace solution | 1 mL |
| Glycerol | 7.5 |
| NaCl | 0.05 |
| CaCO₃ | 0.05 |
| KH₂PO₄ | 0.25 |
| K₂HPO₄ | 0.5 |
| MgSO₄ × 7 H₂O | 0.1 |
| Yeast Extract | 1.35 |
| N-Z Amine A | 2.5 |
| Malt extract | 5.85 |
| L (−) Asparagine × 1 | 1 |
| H₂O | |
| Soy protein | 2.5 |
| Starch | 7.5 |
| Glucose | 7.5 |

Adjust to pH 7

(b) Production Medium A

| | Concentration |
|---|---|
| Substance | [g/l] |
| Agar | 1 |
| Trace solution | 1 mL |
| Glycerol | 7.5 |
| NaCl | 0.05 |
| CaCO₃ | 0.05 |
| KH₂PO₄ | 0.25 |
| K₂HPO₄ | 0.5 |
| MgSO₄ × 7 H₂O | 0.1 |
| Yeast Extract | 1.35 |
| N-Z Amine A | 2.5 |
| Malt extract | 5.85 |
| L (−) Asparagine × 1 | 1.0 |
| H₂O | |
| Soy protein | 2.5 |
| Starch | 7.5 |
| Glucose | 7.5 |
| Antifoam emulsion | 0.2 mL |

Adjust to pH 7

(c) Trace Solution

| | Concentration |
|---|---|
| Substance | [g/l] |
| ZnSO₄ × 7 H₂O | 4 |
| Boric acid H₃BO₃ | 0.1 |
| FeSO₄ × 7 H₂O | 5 |
| KJ | 0.05 |
| CoCl₂ × 6 H₂O | 2 |
| CuSO₄ × 5 H₂O | 0.2 |
| MnCl₂ × 4 H₂O | 2 |
| H₂SO₄ 95-97% | 1 mL |

A frozen suspension (1.5 mL) of a host cell as described in example 3 is inoculated into a two liter non-baffled shake flask containing 500 mL of seed medium. The flask is incubated for 3 days at 30° C. on a rotary shaker at 200 rpm and with 50 mm amplitude. The second seed stage is developed by inoculating 40 mL each, of the first stage seed into eight two liter non-baffled shake flasks each containing 500 mL of seed medium. The flask is incubated for 2 days at 30° C. on a rotary shaker at 200 rpm and with 50 mm amplitude. A third seed stage is developed by inoculating 4 liters each, of the second stage seed into two 150 liter scale stirred tank fermentors containing each 100 liters of seed medium. The 150 liter scale fermentors are operated for 3 days with the following parameters: Temperature=30° C., agitation=80 rpm, airflow=25 slpm, and pressure=0.5 bar. Excess foam formation is prevented by controlled addition of silicon oil-based antifoam agent. pH is monitored but not controlled.

A 5500 liter scale stirred tank fermentor containing 3500 liters of production medium A is inoculated with 200 liters from the third seed stage. Operating parameters of the 5500 liter scale fermentor are: Temperature=30° C., airflow=1050 slpm, and pressure=0.5 bar. Agitation is controlled at 60 rpm and, after 44 hours, increased to 80 rpm. Excess foam formation is prevented by controlled addition of silicon oil-based antifoam agent. pH is monitored but not controlled. The fermentor containing 3500 liters of broth is harvested after 5 days of incubation.

### 7. Isolation of Thiopeptide Derivatives

The fermentation broth are harvested and extracted over night in a stirred tank by addition of ethyl acetate. During extraction the mixture is passed through a continuous Dispax® reactor (Jahnke & Kunkel, Germany) for maximum sheer force and optimal mixing. After separating the two phases on a continuous Westfalia separator SA20 (Westfalia Separator AG, Oelde, Germany) the ethyl acetate phase is concentrated by evaporation under reduced pressure. During evaporation a precipitate is formed which is separated by filtration.

The precipitate obtained from the extraction of the culture broth according to the procedure described above is dissolved in Dioxane/water 95:5 and filtered to remove insoluble ingredients. The filtrate is concentrated under reduced pressure in the presence of diatome 8 (Isolute®, International Sorbent Technology Ltd., Hengoed Mid Glam, UK) The obtained powder is applied to a chromatographic column of silica gel (e.g. 0.040-0.063 mm, column size 5×25 cm) prepared in dichloromethane/methanol/acetic acid 90:10:0.5. The column is developed with a mixture of dichloromethane/methanol/acetic acid 90:10:0.5 at a flow rate of 35 mL/min. Fractions of 30 mL are collected, which are analyzed by HPLC. To the pooled fractions containing compound I 20 mL Isopropanol is added and concentrated under reduced pressure until the compound precipitates from the remaining Isopropanol. After separating of the solvent from the precipitate through centrifugation the residue is dried under reduced pressure yielding semi-purified thiopeptide derivatives.

1. A thiopeptide precursor protein comprising an amino acid sequence selected among the group consisting of
i. SEQ ID NO:1;
ii. SEQ ID NO:5;
iii. SEQ ID NO:11; and
iv. or a variant of said amino acid sequences having no more than 1, 2, 3, 4, 5, 6 or 10 deleted, inserted and/or substituted amino acids when compared to SEQ ID NO:1, SEQ ID NO:5 or SEQ ID NO:11.

2. The thiopeptide precursor protein of claim 1, wherein said precursor is a biosynthetic precursor for an Ef-tu thiopeptide inhibitor.

3. The thiopeptide precursor protein of claim 2, wherein said Ef-tu inhibitor is selected among the group consisting of GE2270A, GE37648A, Amythiamicin or a compound as represented in any of Formulas I-XI below:

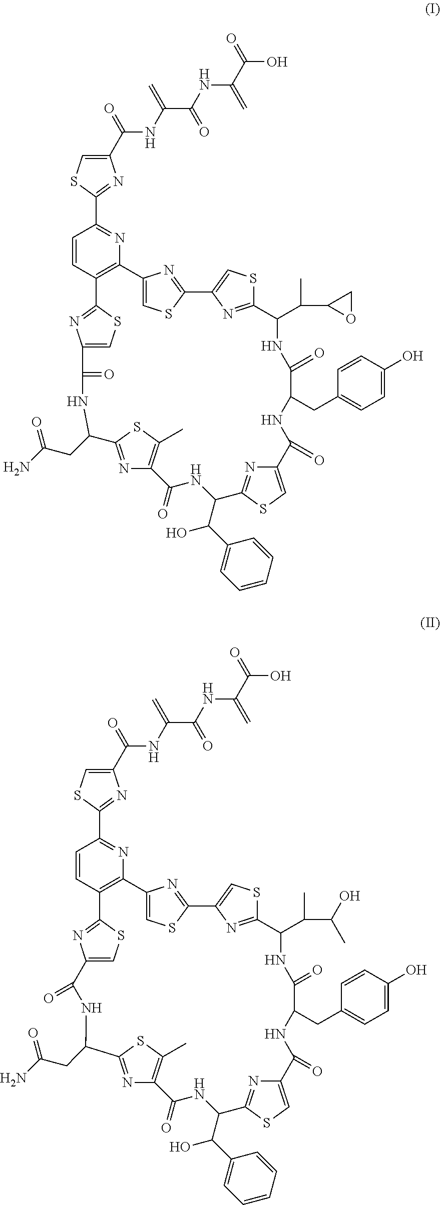

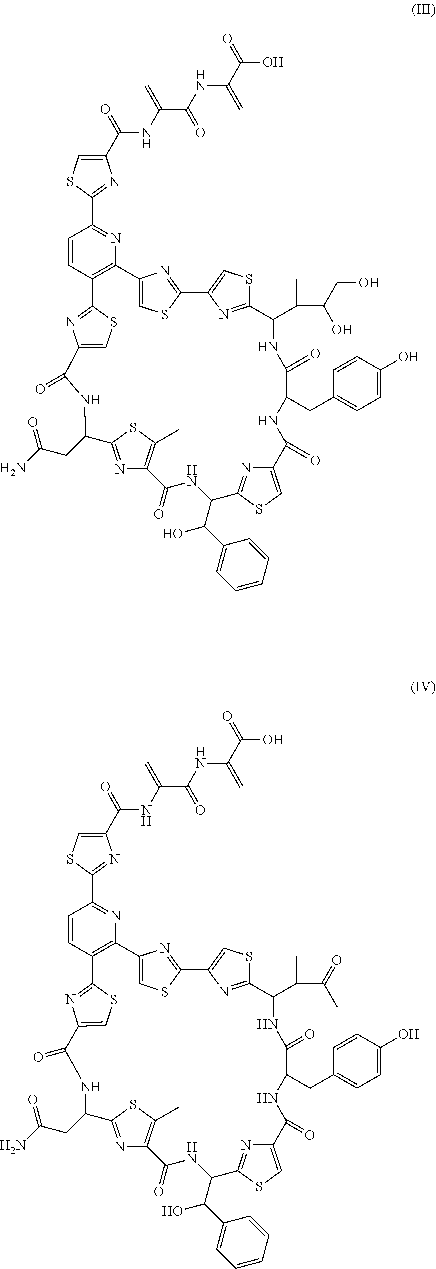

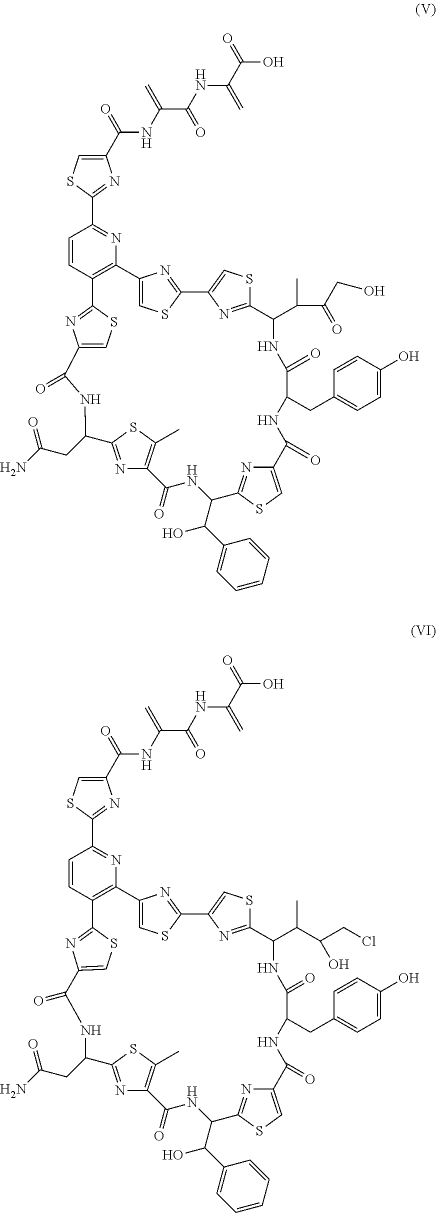

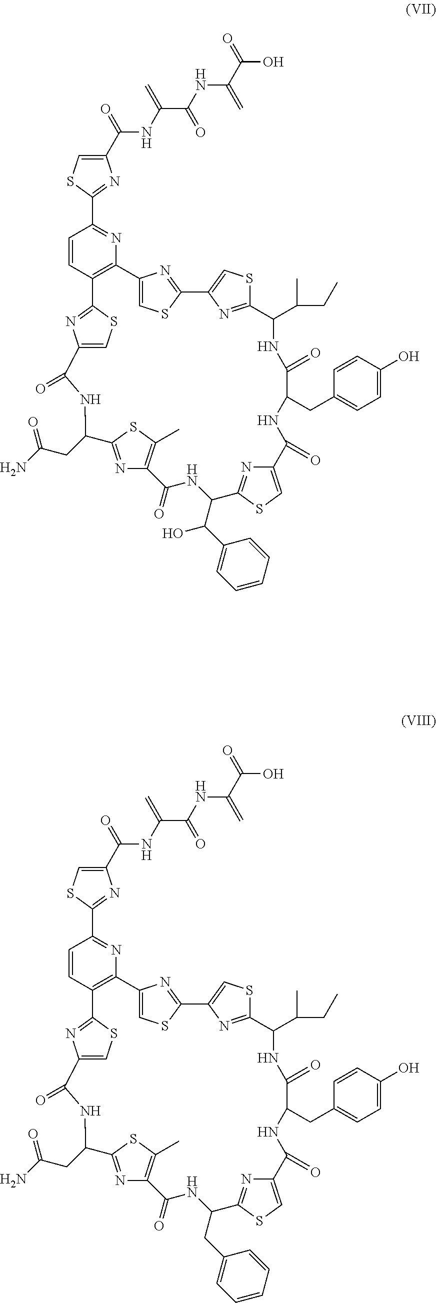

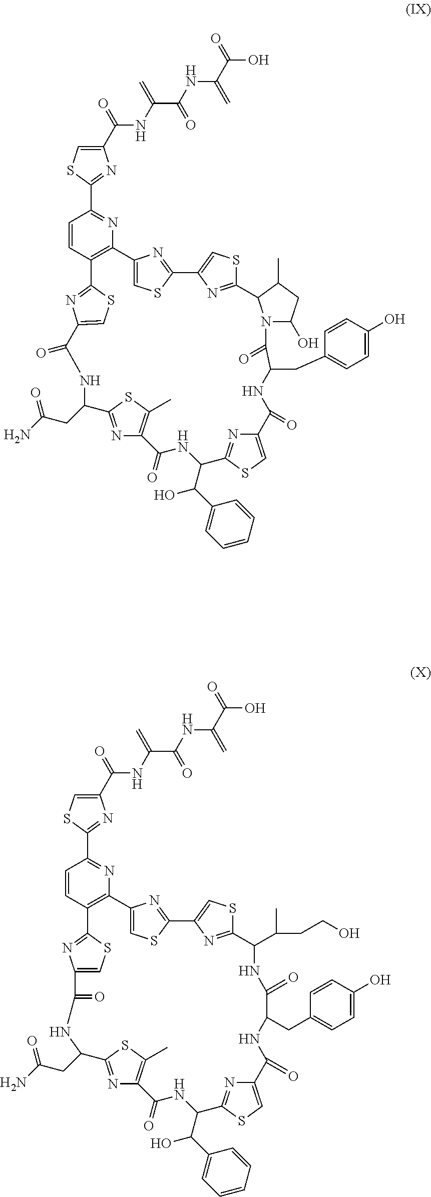

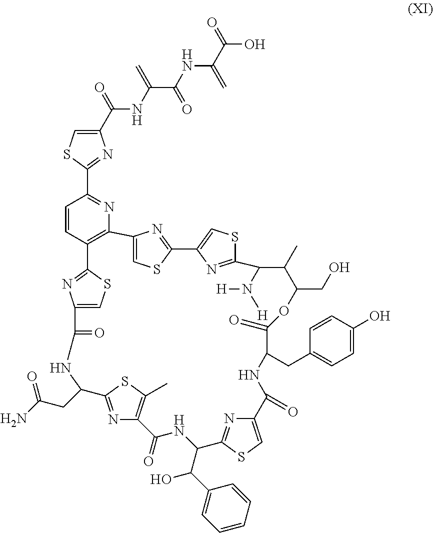

4. A nucleic acid comprising the nucleotide sequence encoding the thiopeptide precursor protein according to claim 1.

5. The nucleic acid of claim 4, comprising the nucleotide sequence of SEQ ID NO:5.

6. The nucleic acid of claim 5, comprising the nucleotide sequence of SEQ ID NO: 6 or any fragment thereof comprising at least SEQ ID NO:5.

7. The nucleic acid according to claim 4, wherein said nucleic acid sequence is operatively linked to heterologous transcriptional and translational control sequence.

8. The nucleic acid of claim 7, wherein said nucleic acid is an expression vector.

9. A polypeptide for the biosynthesis of thiopeptide, comprising an amino acid sequence selected among the group consisting of:
i. any one of SEQ ID NO:23-34 and
ii. a variant of an amino acid sequence listed in (i), having no more than 1, 2, 3, 4, 5, 6 or 10 deleted, inserted or substituted amino acids when compared to the corresponding wild type amino acid sequence listed in (i) and retaining substantially the same enzymatic function.

10. A nucleic acid comprising a nucleotide sequence encoding a polypeptide of claim 9.

11. The nucleic acid of claim 10, wherein said nucleic acid sequence is operatively linked to heterologous transcriptional and translational sequence.

12. A host cell comprising the nucleic acid according to any of claim 4, wherein said nucleic acid is not naturally found in the genome of said host cell.

13. The host cell of claim 12, further comprising other genes required for thiopeptide biosynthesis.

14. The host cell of claim 13, wherein said other genes required for thiopeptide biosynthesis comprises a nucleic acid comprising a nucleotide sequence encoding a polypeptide for the biosynthesis of thiopeptide, comprising an amino acid sequence selected among the group consisting of:
i. any one of SEQ ID NO:23-34, and
ii. a variant of an amino acid sequence listed in (i), having no more than 1, 2, 3, 4, 5, 6 or 10 deleted, inserted or substituted amino acids when compared to the corresponding wild type amino acid sequence listed in (i) and retaining substantially the same enzymatic function.

15. The host cell of any of claim 13, which is selected among the group of species consisting of Nonomuraea sp., Planobispora sp., Amycolatopsis sp., Escherichia coli, Corynebacterium sp., Bacillus sp. and Streptomyces sp. such as Streptomyces lividans, Streptomyces coelicolor, Streptomyces albus, Streptomyces ramocissimus Streptomyces collinus, Streptomyces fradiae, Streptomyces azureus or Streptomyces griseus, and wherein said host cell is rendered resistant to said thiopeptide.

16. A mutant microorganism, wherein said mutant microorganism no longer express a gene encoding the thiopeptide precursor protein comprising an amino acid sequence selected among the group consisting of
i. SEQ ID NO:1;
ii. SEQ ID NO:5;
iii. SEQ ID NO:11; and
iv. or a variant of said amino acid sequences having no more than 1, 2, 3, 4, 5, 6 or 10 deleted, inserted and/or substituted amino acids when compared to SEQ ID NO:1, SEQ ID NO:5 or SEQ ID NO:11
when compared to the corresponding wild-type microorganism.

17. The mutant microorganism of claim 16, wherein said mutation is a disruption of the gene encoding the thiopeptide precursor protein comprising an amino acid sequence selected among the group consisting of
i. SEQ ID NO:1;
ii. SEQ ID NO:5;
iii. SEQ ID NO:11; and
iv. or a variant of said amino acid sequences having no more than 1, 2, 3, 4, 5, 6 or 10 deleted, inserted and/or substituted amino acids when compared to SEQ ID NO:1, SEQ ID NO:5 or SEQ ID NO:11,
in the corresponding wild-type microorganism.

18. The mutant microorganism of claim 17, wherein said microorganism is a Nonomuraea sp. and said mutation is a disruption of the gene comprising SEQ ID NO:15 or SEQ ID NO:16.

19. The mutant microorganism of any of claim 16, wherein said mutant is further transformed with a nucleic acid comprising the nucleotide sequence encoding the thiopeptide precursor protein comprising an amino acid sequence selected among the group consisting of
i. SEQ ID NO:1;
ii. SEQ ID NO:5;
iii. SEQ ID NO:11; and
iv. or a variant of said amino acid sequences having no more than 1, 2, 3, 4, 5, 6 or 10 deleted, inserted and/or substituted amino acids when compared to SEQ ID NO:1, SEQ ID NO:5 or SEQ ID NO:11.

20. A mutant microorganism, wherein said mutant microorganism no longer express one or more genes encoding one or more polypeptides as defined in claim 9.

21. A polypeptide for the biosynthesis of thiopeptide, comprising an amino acid sequence selected among the group consisting of:
i. any one of SEQ ID NO: 47-60,
ii. a variant of an amino acid sequence listed in (i), having no more than 1, 2, 3, 4, 5, 6 or 10 deleted, inserted or substituted amino acids when compared to the corresponding wild type amino acid sequence listed in (i) and retaining substantially the same enzymatic or regulatory function; and
iii. a variant of an amino acid sequence listed in (i), having at least 80% or at least 90% identity with one of the polypeptide listed in (i) and retaining substantially the same enzymatic or regulatory function.

22. A nucleic acid comprising a nucleotide sequence encoding a polypeptide of claim 21.

23. The nucleic acid of claim 22, wherein said nucleic acid sequence is operatively linked to heterologous transcriptional and translational sequence.

24. A host cell comprising the nucleic acid according to claim 22, wherein said nucleic acid is not naturally found in the genome of said host cell.

25. A host cell according to claim 24, further comprising other genes required for thiopeptide biosynthesis.

26. The host cell of claim 25, wherein said other genes required for thiopeptide biosynthesis are selected from the group consisting of
a. a nucleic acid encoding a thiopeptide precursor protein comprising an amino acid sequence selected among the group consisting of
i. SEQ ID NO:1;
ii. SEQ ID NO:5,
SEQ ID NO:11; and
iv. or a variant of said amino acid sequences having no more than 1, 2, 3, 4, 5, 6 or 10 deleted, inserted and/or substituted amino acids when compared to SEQ ID NO:1, SEQ ID NO:5 or SEQ ID NO:11; and
b. a nucleic acid comprising a nucleotide sequence encoding a polypeptide for the biosynthesis of thiopeptide, comprising an amino acid sequence selected among the group consisting of:
i. any one of SEQ ID NO:23-34, and
ii. a variant of an amino acid sequence listed in (i), having no more than 1, 2, 3, 4, 5, 6 or 10 deleted, inserted or substituted amino acids when compared to the corresponding wild type amino acid sequence listed in (i) and retaining substantially the same enzymatic function.

27. A mutant microorganism, capable of producing thiopeptide that is deficient in the expression of one or more of a tailoring gene encoding one or more polypeptides as defined in claim 21.

28. A method for producing a thiopeptide precursor protein, said method comprising the step of culturing a host cell according to claim 12 or 24 under conditions appropriate for production of said thiopeptide precursor protein.

29. A method for producing thiopeptide compound, said method comprising the step of culturing a host cell according to claim 12 or 24 under conditions appropriate for production of said thiopeptide compound.

30. The method of claim 28, further comprising isolating said thiopeptide precursor or thiopeptide compound in a substantially pure form.

31. The method of any of claim 29, wherein said thiopeptide compound is selected among the group consisting of: GE2270A, GE37648A, amythiamicin, micrococcin, thiostrepton, nosiheptide, thiocillin, thiocins, nocathiacins, berninamycin, A10255B and radamycin and a compound as represented in any of Formulas I-XI below:

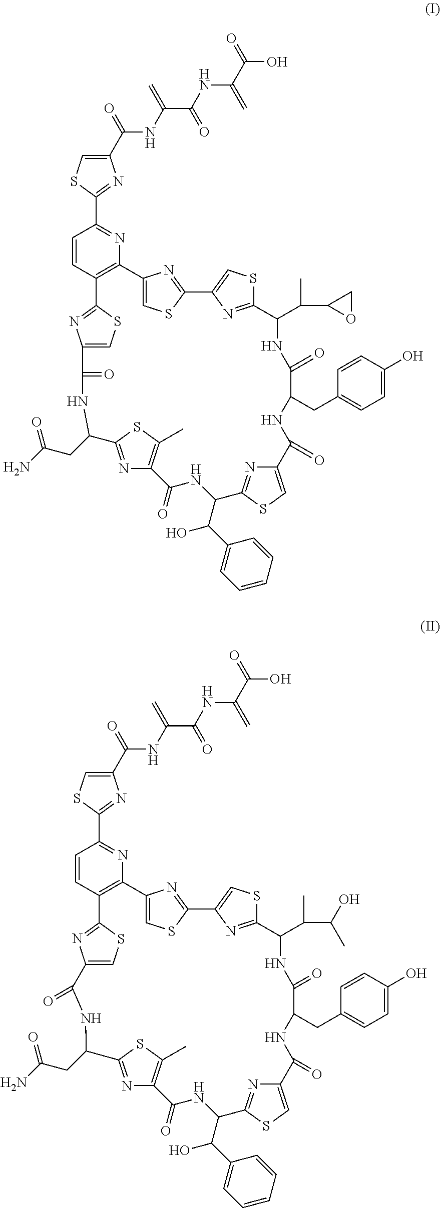

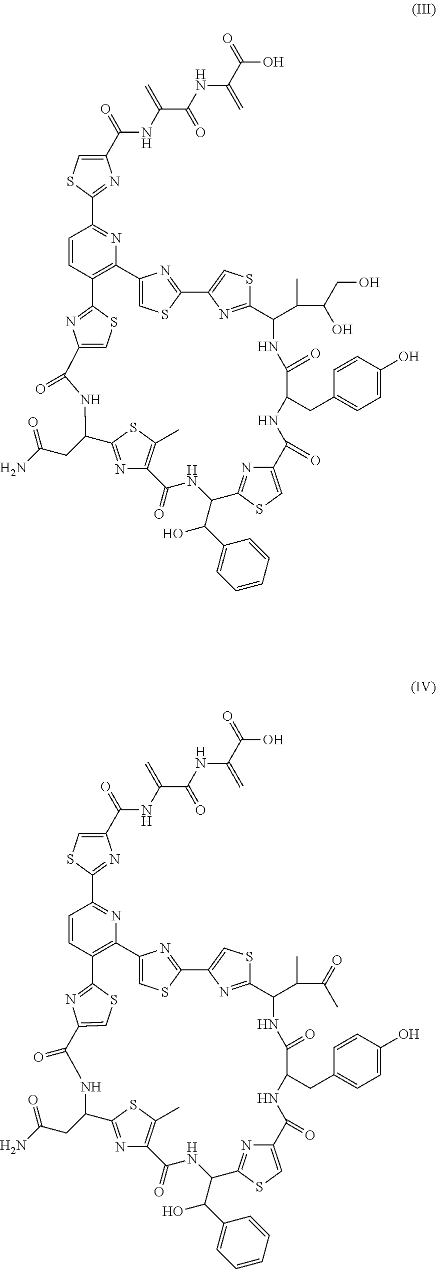

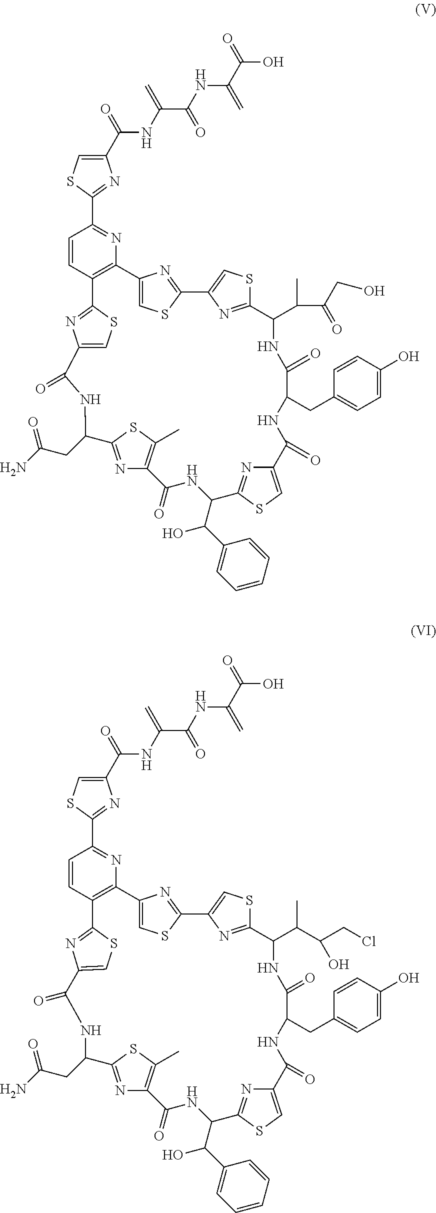

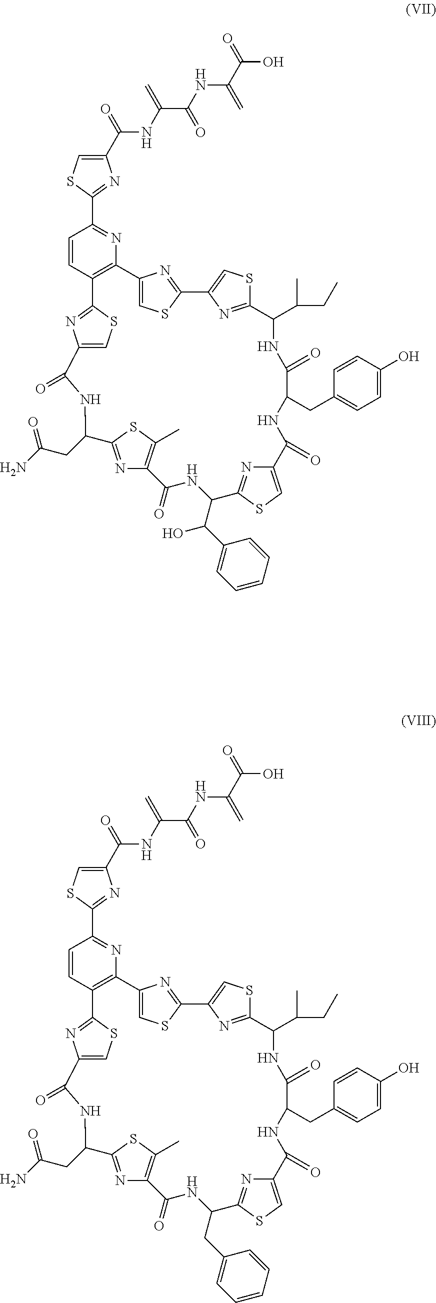

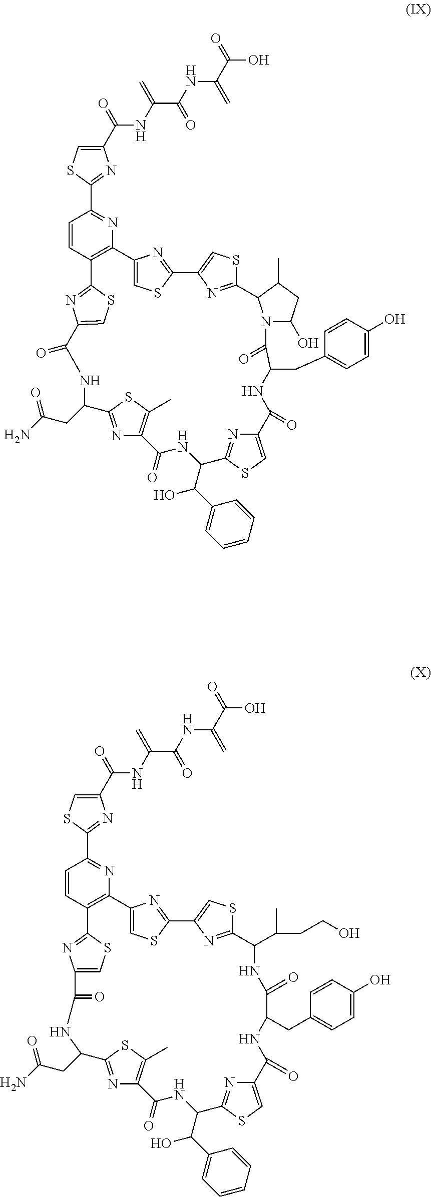

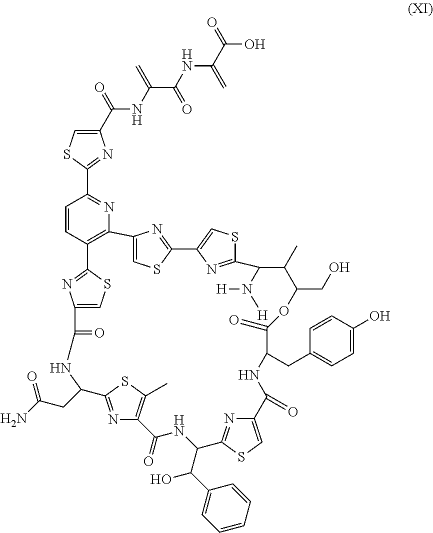

32. A method of producing thiopeptide derivatives, comprising
i. synthesizing an altered thiopeptide precursor in a host cell by gene expression of the sequence encoding said altered thiopeptide precursor in said host cell,
ii. synthesizing said thiopeptide derivatives from said altered thiopeptide precursor.

33. The method of claim 32, wherein said altered thiopeptide derivative precursor is a variant of SEQ ID NO:1, SEQ ID NO:5 or SEQ ID NO:11.

34. The method of claim 32, wherein step ii) is performed in vitro.

35. The method of claim 32, wherein step ii) is performed in vivo in the same host cell as step i).

36. A method of producing thiopeptide or thiopeptide derivatives, comprising
i. providing a culture medium comprising a thiopeptide precursor protein as defined in claim 1,
ii. culturing microorganisms in said culture medium, wherein said microorganism further comprises other genes required for thiopeptide biosynthesis.

37. The method of claim 36, wherein said other genes required for thiopeptide biosynthesis are selected among those encoding a polypeptide selected among SEQ ID NO:23-34 and SEQ ID NO:47-60.

38. The method of claim 36, wherein said microorganism is selected among the group of species consisting of Nonomuraea sp., Planobispora sp., Amycolatopsis sp. and Streptomyces sp. such as Streptomyces lividans, Streptomyces coelicolor, Streptomyces albus, Streptomyces ramocissimus Streptomyces collinus, Streptomyces fradiae, Streptomyces azureus or Streptomyces griseus, wherein said microorganism is a strain selected for resistance against said thiopeptide or thiopeptide derivative


SEQUENCE LISTING (65 sequences)

SEQ ID NO: 1 - protein, 14 aa, nonomuraea sp.
SCNCVCGFCCSCSP

SEQ ID NO: 2 - protein, 14 aa, nonomuraea sp.
SSNCFCYPCCSCSS

SEQ ID NO: 3 - protein, 14 aa, nonomuraea sp.
SCNCFCYICCSCSS

SEQ ID NO: 4 - protein, 14 aa, nonomuraea sp.
SCNCVCGVCCSCSP

SEQ ID NO: 5 - protein, 13 aa, nonomuraea sp.
SCTTCVCTCSCCT

SEQ ID NO: 6 - protein, 17 aa, nonomuraea sp.
IASASCTTCICTCSCSS

SEQ ID NO: 7 - protein, 12 aa, nonomuraea sp.
SCTTCECCCSCS

SEQ ID NO: 8 - protein, 13 aa, nonomuraea sp.
SCTTCVCVCSCCT

SEQ ID NO: 9 - protein, 13 aa, nonomuraea sp.
SCVGSACASSSSS

SEQ ID NO: 10 - protein, 12 aa, nonomuraea sp.
  misc_feature (6)..(6): Xaa can be any naturally occurring amino acid
SCTTCXCXCSCS

SEQ ID NO: 11 - protein, 15 aa, nonomuraea sp.
SCTTSSVSSSSSSSS

SEQ ID NO: 12 - protein, 16 aa, nonomuraea sp.
SCTTSGCTSSSSSSSS

SEQ ID NO: 13 - protein, 17 aa, nonomuraea sp.
SCTTSGSACSSSSSSSS

SEQ ID NO: 14 - protein, 15 aa, nonomuraea sp.
SCVGSACACSSSSSS

SEQ ID NO: 15 - DNA, 42 bp, nonomuraea sp.
tcctgcaactgcgtgtgcggcttctgctgctcctgcagcccg

SEQ ID NO: 16 - DNA, 42 bp, nonomuraea sp.
tcgtgtaactgcttctgctacatctgctgctcctgctcgagc

SEQ ID NO: 17 - DNA, 174 bp, nonomuraea sp.
atgagcgagctggaatccaagctgaaccttagcgacctgcccatggacgtcttcgagatggccgacagcggcatggaggtcgaatccctcaccgcaggccacgggatgcccgaggtaggcgcttcctgcaactgcgtgtgcggcttctgctgctcctgcagcccgtccgcgtag

SEQ ID NO: 18 - DNA, 150 bp, nonomuraea sp.
atggacctcagcgatctgcccatggacgtcttcgaactcgccgatgacggtgttgcggtcgagtcccttactgctggtcacggcatgaccgaagtgggagcttcgtgtaactgcttctgctacatctgctgctcctgctcgagcgcctga

SEQ ID NO: 19 - protein, 57 aa, nonomuraea sp.
MSELESKLNLSDLPMDVFEMADSGMEVESLTAGHGMPEVGASCNCVCGFCCSCSPSA

SEQ ID NO: 20 - protein, 49 aa, nonomuraea sp.
MDLSDLPMDVFELADDGVAVESLTAGHGMTEVGASCNCFCYICCSCSSA

SEQ ID NO: 21 - protein, 414 aa, nonomuraea sp.
MPTTLPKTLRGSDGALAEQPLLIYVNVPFCNSKCHFCDWVTEVPLADLRLTPDSSPRRRYVAALVKQIETHAPTLGGLGYRPEVMYWGGGTASILTIEEIEAVAGALAARFDLGGLVEATIEGSPESMDPGKLKLFRAIGFNRISIGVQAFDDARLRRIGRVHSAEQAERAVRMAADAGFDNINIDLIVGFPGQGADEVSRTIQHAVTLPVNHFSVYPYRPTGGTVMRRQVGRGMGRIDVEEQLRSYDHARDLLARHGFEEYATAYFGAPRCESDEVYYKLTMDWIGFGSGANSLIGTRFLLNDRGGLHRFSTAPHRFDSDTPASAPHLTRHFLAQALTTVDGMDARTFQLRTGRSLRAACEEPAVRRMLEQINRRGRLIVDSRGIRLHRDDMASAYITMNSVDLYAATEQPGG

SEQ ID NO: 22 - DNA, 1242 bp, nonomuraea sp.
atgccgaccacactcccgaagaccctgcgcgggtcggacggcgcgctcgccgagcagccgctgctcatctacgtgaatgtgccgttctgcaactcaaaatgtcacttctgcgactgggtgaccgaggttccgctggcggatctgcggctcacgccggactcgtcgccacgccgccgctacgtcgccgcgctggtcaagcagatcgagacgcacgcgccgacgctgggcggactcggctaccggcccgaggtcatgtactggggcggcggcacggccagcatcctcaccatcgaggagatcgaggcggtggctggggcgctggcggcgcggttcgacctcggcggcctggtcgaggccaccatcgagggcagcccggagtccatggaccccggcaagctgaagctgttccgtgcgatcgggttcaaccggatcagcatcggggtgcaggcgttcgacgacgcgcgcctgcgccgcatcggccgggtgcacagcgccgagcaggcggagcgcgcggtccggatggccgccgacgccggcttcgacaacatcaacatcgacctcatcgtcgggttccccgggcagggcgccgacgaggtgtcgcgcacgatccagcacgcggtgacgctgccggtcaaccacttctcggtctacccctaccggccgaccggcggcaccgtgatgcgccggcaggtgggccgcggcatgggccggatcgacgtggaggagcagctccggtcgtacgaccacgcccgcgacctgctcgcccggcacgggttcgaggagtacgccaccgcctacttcggcgctccccgctgcgagtccgacgaggtctactacaagctcacgatggactggatcgggttcggctccggggccaactccctcatcgggacccgcttcctgctgaacgaccggggcgggctgcaccgcttcagcacggccccgcaccgcttcgactccgacaccccggcctcggccccccacctgacacgccacttcctggcccaggcgctgaccacggtcgacggcatggacgcgcgcaccttccagctgcgcaccggccgttcgctgcgggcggcgtgcgaggagcccgcggtgcgccggatgctcgaacagatcaaccgccgcggcaggctgatcgtcgactcacgcggcatccggctgcaccgcgacgacatggcctcggcctacatcaccatgaacagcgtcgatctgtacgccgcgaccgagcagcccggcgga

SEQ ID NO: 23 - protein, 827 aa, nonomuraea sp.
VPPAQAGGTPPGVRRQIMEVGREIVVRVAGLPAAVLADLRLPHTAELVTHLSAERRRLAAEAAALSGELFDLIGAAGSARAALVGLRRALAPGHRPPSPRLIGLCPLPAPLAERVTAWVRARYDWEDRRRDLAGTLDKERADALDRVRAACVAPAFRRGLALSGGELTSTLERWLADPGRAPRQGKVLRLVKYLTRAAAKTSPYGSFMVSALAGRPGDDPPIPELLTVAEPPGAFLDAVGDALLADPALAGQVPLRPNPSLTWTAQGLLFVRTVRAAAGEQAGPKEEIATAGRAAALELCLRHAESRPTAPQLAELLAEAGADPGEAAAFVDRLVAAQLLLPCPPVHDDDPDPFGAWARQVCAPELRELAAASRPVAAAVDSPGQRRARIAEAAAAVADRLGIDPPADPAHEHDVSTGRPAPPPLPAGVLADLDAVRRWLAVFDWKVPVRVGVGAFCRERFGSGSRTPFLEACRAATAALPHLFGPAAMPWFLDLTGDGRLRELDRLRTRARDLARSGVLDRRRVLADTADWPAWLTWPASTGFYLQTLPGGVVLNAVHAGHGRAAGRVHHLLARAGAAPPRPPRSGLPRAEFGGRFGSALNTRTPSTRYEIDHPGATSGRDPRHRVPLGTLMVVHDPDTDLVHLHSDRLGRVEPVHLGMMGELGLPAVAGFLERAFAPTYLFHPSVPPFISLRDLAGASSARRFPRVSVGDVVVQRARWTVPAGLVPARTGADGDHLLALAEWRREHGIPERCFVRGWKPGAALGKARKPLYVDFASWHLVALFEREARTNAALVIDEALPDPLADGAPAHVTEYHVEIGDREREQ

SEQ ID NO: 24 - protein, 299 aa, nonomuraea sp.
MTERPGWFAAHVHYRGDLDALLREAVAPLVRALAADFFFLRYWDGGSHLRLRLRGGDQVAVARHLDAYLAAHPAPETTSQEEYARVAPVLAAREGMTGHLTTLRPNNTVEFAAYRPETAKYGTGDALRAVERHFVESSRYALDVLDRRPTGNQRELAVLGVLLLAWYAARPLDEPPGEPVDDELIGAVEALCRGWRGGRDLPEELVAEEYGRVRERVAGLAGALRDLVPDPDAPGSSMRAWAATFDRLAAALPPPDRLRVLDTCAHLAANRLGVSMAAEVRLRLLAARALREVAPVGRR

SEQ ID NO: 25 - protein, 333 aa, nonomuraea sp.
MSWRRVDVAYHDPDLDGLILATRPLLAGTPGRGWFQRHWVRGPHLELWFDAAQPSWERIRDVLEPWLRVNPSRARIDRDRLLAQHRHLAAAERIDEPLLPFYADNTLHRAAPRSRAHVLGGPAAEELFHDFHTTASAVAYDELDAVRAGESRLVMALDLMVAAAHAHAEGGVRGGFVSFRSHAEAFLASAPGLRERWDAEYAARAGALRARITAVVAGIPRGRAWAGLLDRFADRGDELIASGALLVEPAGPDAVARPDTAFHRALRGNRTWHEEVLRSAPFRRYRLLLNLTYLQLSRLGVNAVQRALLCHFAASAVEQEYGVSAIEIAMGGA

SEQ ID NO: 26 - protein, 455 aa, nonomuraea sp.
VMSVQVEPAVTHRWALRPGVYRATAPGGDLMLAAWPHATPLGRATPELLALLDGLAAGSLPLDEPGDLAPAVELLRAGGWLRKTVAYAGRDLYTITPVAAPTDAPPPPRDEPALSRFALLRNTADGLVLEMPGSWCDVHVHDPAVAALLADPSGDGGLPAEAAAALRADLAAAGMLMEREEPFERRQWSTHELWFHERSRLGNRGWFGAHFGGTFWARGRHDPPPARPSPYPGPAVELARPDLAALRRTDPTLTAVLEDRESVRDHDDDDPISAEQLGELLYRCARVRLLRHLEGFEYAGKPYPAGGSAYELEVYPVVRLARGLDPGMYHYDAHDHRLRQVRPAGHPAVRRMLKVATESSVAKAPPQVLLVISARVGRILWKYEAMGYALMLKHVGVLQQTLYAVATAMDLAPCALGSGDDLAFTAATGRDRLVECAVGEFMVGSRRKEPAAWEL

SEQ ID NO: 27 - protein, 608 aa, nonomuraea sp.
MGALNLLLRPDAHYAEVDGGVYVLSHQGETFISGTSVHRWLDRLAPLLDGTRTLDQLTAGLPADRAGFVARLVGTLTERGLVREVGPAVSDTLTDAERGEHRALLAYLGYFHDSPGRVLEDVRDTPTAIVGSGPLAAAVVRACAAAGLRRVEIGEEAGAARVVVHVAERAEPERAARLERRCAAEGVLLAQVMPGPDGIWWQPAARGGCWASAWRRHRALAGVDRTGTPLDPVAVAVVAGQVAHDVFRVLAGLREEAPPRLVVLDPRTLASTAHPVVAHPFELPAAPLDEAAFLDRVAALRAAPALSEAEFSRRAKGLMDSTVGLFAEIDEGDLAQLPLHVTATTVADPCGLIGSAPRPVVTGAGLTFEEARYRAARAALALAGTLALDRRRLVDGRVWAYDLADGAARLVPADEVFASTAGGSTAGVPLGAAAAYSWAEAVAEGLVAQAAALTLAGAGHAGEPYGRADLAGAPAYLLAMVRALGEELTVHDVTGPLGVPVVVGTLSGGATACGAGMTTDAAVTACLRDLLLLRQAEINDQPVYAPPSGRPLPGHLRGNHPAPAVPGTDADRLAARLAERGHRPLAVPLDHDQAVHAVLPFAVRVVCR

SEQ ID NO: 28 - protein, 578 aa, nonomuraea sp.
MIRLPDHGELAVPIGDGPMLPLRTELGRVVIGPLERPGVPGCRACVRIRAARVQPDARKAEAVRARHAPAPSQWLTPLATDLVRVLVASESAALAADTDPRTAGAVLEIDLATLEITRHRFLPDPLCPHCGGLPPDGPAELTLRPRRKLGGSPRTRPVELNALLETYVDGRVGMIRPLKSGVQGGLTVASAMLPIRAGHGLEPGVGRTRSYTASRLVAVLEALERYGGVSPGGRRTSVTAAYRDISGHAVHPDTFGTHPEENYDRPGFAFRRFTEDTVCRWVWGYSFAKAQPVLVPENQVYYYARNLPDGEQPFVFEVSNGCALGSCLEEAILHGLLEVVERDAFLLTWHARREVPVLNPALASDPVLPMQAAAITAETGHRVLCFDTTAEHGIPSVWAMAVDVEHRSDRPATAHAAGAALTLEQAAMNALSELGPLLADVIRRYPEERERAEAMVRDPEQVATMHDHSLLYAVPAAAERLSFLTGRADGPRTGFGSARFTGDDLTADLRAMIDAVIDAGMDVVVVDQTTPEHLAGGFRCVKVLVPGALPMTFGHQYRRLDNLPRLAAARTTDPHPFP

SEQ ID NO: 29 - protein, 857 aa, nonomuraea sp.
MAVIEREESDGPPVAVRTCGVAAAELESLRWDESYALVTRLLAETARVAADGRRLAVALGDVIGGLGPSGSRPVLVGLRRALHTGRLPSGREWGPEAGAALPYGLRGEVEEWVRRARECAGLRARLPEVVARESLEKEERLRAAAADPAFRRGLALAGGELAVDLETWLADPARRPKPQKLLRLAKYLVRAAVKTSPYSTFTSTGRAVWGGGERHVERVVPVLELDGVQTFSDDRVRVNPSVTVAGGTAEFIGPPPGEALVSIGVTEAVAACLRVAEGGEWVPRGRFAEALGAEPAAVTKFLDKLLAVGLLEARPDGDPPPHLLDGIRRTERATDPSTFRHELGRLIAAARAAARATAPGADTAKNAAPAGRATRGDGGADAERATRGDSDADDGRATRGDGGADDRGAARGDGGVGRAVVHEVAVCVEPVARLDPARWRGGLADLDVVRRWLAVFDAKHPMRLAVAAYLTTRYGPDPAVPFLTLHRHIQRELAGTGAAGRELRAFLGSSAAWTQPLGTSALPRIRELERLRAEARSLALDAEDPDGTCRVTPGQLAAQLESWPSWIAIPASSACYVQAVAGTLVLNVVHGGHGRALRRLDHLLERAGGHPEPPRIEDPDGAVYAEFSGDLGSTLNARPPSTRYEIDYPHSPGTRPPDLRLPLTDLHVTLCPDSGLPELRSKRLGRRVVPLHLGLAAEFRLPPAARFIERVFGPGYLLHPSSPPLVRMGRVPSEVTRYPRVEAGRVVVQRRRWLAPAATLPVRAKGEGDAAYLTRLIAWADEYGVPHRSFVRAWPEQTGDKGQDKARKPLFLDLANLFLVKNFERQIRGCAFALFEEALPDPGPERVTEYLIEVGGR

SEQ ID NO: 30 - protein, 271 aa, nonomuraea sp.
MSWVSVHVFSQGPLDDMITGLAGPLLGDDGFFLRYWEGGPHLRIRVRSHPAEQVVERAEAYLREHPSEAVIDPEEYARLAATLAAREGVPGYTRRLYPNDSAHLIPYQPEHDRYGTGRSLDAVERHFHDSSVIALQLIGAGLGHERRSMAWLTMLLVAWQVAGEGRAFGPPADAGVQVPHERMAALVEHARAVTQEPAPSEPRGVVAAWYASIARLVEELAAAGFDAARTGATVDLAAHLLANRLGIRVQEEARLRHLASGATRESEVGVG

SEQ ID NO: 31 - protein, 319 aa, nonomuraea sp.
MTWTRLRVDYHDGPMDDLILDALRPAWHEIRGYFLRHWVCGPHLRIFVDGDATPIVRAVERHLAACPSAGTTDPQALLPLHERLAELEGERGPLLPWAPDNTVTAEPPGLDTELDRFLADFYADTTEAAFDALGRVRAGTPLPGIAFDLVVATAHDLSEGGLPTARTSLRSHAEAYLSRLPGGVRAKWQAHYERNQEPLTARIKALTGAGEPGAWLRTIRATRDRGRTLIDEGRLSLGYATDGPSTRPPLAAVSPFHRNLETDERWLALKDTPAFAAYRLALNCAYLHLTRLGLTPDQRFLICHLAASAADDVYRAVAS

SEQ ID NO: 32 - protein, 428 aa, nonomuraea sp.
MTGERYELRAGVHLARLDAGVRLFAWPHAETLGELTRDELDLLRDLAAGPRTAGGPLVERLRAGGWLKITITRDGVPLYTLAPLRPPPPRPAVPERPVLSRFATLRREGDALVAGSPLAWCDLYVHDPGLVGDVVRPGGEVLADLVWAGLAVPEEAEDRELRFRQWAPHELEFHDRSRVGHRGYLGDGFGGTFWARGRFDPLPARPRPYPGRAIELPTPDLDELRLKDPSLTDVLEGRRSVREHDDERPITLDLLGELLYRTARVRECWTADGVEYTRKPYPSGGSVYELEIYPVVRNVAGLEPGMYHYDGHDHVLRPVREAGHPAVRRLLTVAGHGAATGRRPQVLLAVSSRAGRLLWKYEGMGYALTLKHVGVLYQTLYCVATAMGLAACGLGSGDSAAFAEATGRDPLEECAVGEFMIGTAEAPR

SEQ ID NO: 33 - protein, 630 aa, nonomuraea sp.
LNDFKPRLPADIRFAPTSGGVAFLTREGVVRMNGASVHLWVERLAVHLDGSRTVAELTAELPDERRAFVTSLLTTLRERGLVEDAGERGVLPVAAGTACSLLADADRFGELAPALADALRRLGLEVLTEPSGGALVHVAAADADGLAAAGRLDRLSAERGLPIAHVLLREGEAWWSETGVVGRDVPPWAAGWRRLAAMEAVPRTAGHVSVSPVAAAVVAGQVVADLGRCLSGTPRVSGGPRLHVVDLDGFGMRTHSFLPHLSALPPGTGVSAGPLGEEEFSRRAARLMDSRTGVFAEIEEGEYGQLPLHVSVTTVADPMGALGGALARVIGVGADFATARYRAALKALTVHGLLALDPRRLVTAEGTPLTGAGEPASGFPADPGARERPIGGFSADPGAGERPDDGLLDDPLVGDVAAGRVEAFVRGRDLVEGGEVLLPARAVFLDPHATGAAAGYSAEEALVAGLLDHCAALAGLDGTLPPVDVGGARAADDETARSAALLDAIGLPYQVLDATGPSGVPVHAGTLGGRLVARAAGVTPLAALRATLEGILAAYQGIPGPAPLPDLCPAVAQEPPVVLDTGAAVLGAGELAAALAGAGLRVSAVPLDHDREVHALMPHVVRVVTEPLHG

SEQ ID NO: 34 - protein, 610 aa, nonomuraea sp.
VGTGLLAEEITIALKDRDGRAEVTITACDGWARGPRRDGAAWLPVRVEYGVAVIGPLERPGEPGCVTCFETRRDRVRTDSADRAAVLAVHGPELAGRASPYLGRLAARTIAALAAHAAEGPGCAVWYVDLRTLTAERHTFLPEPHCPDCGDLPDDDRASAAFEPGAAPRPPGYRVRNVPAEHGALLDTYVDGECGLIRPLVRDTLGGLVIAGAMLPLRFEGGSEPGVGRTRGYRASEVTAVLEALERWGGVEPGGKRTRVRASYAEVAADALDPRTLGEHDTSSYEMEGFAFRPFAEDAVCDWVWGYSFARRSPILVPESLAYYYVRGRERPFLFEISNGCALGGSMAEALLYGLLETVERDAFLMTWYGRLPVPRIDPGTARDRTIPLQAAAIIAETGYRVELYDTTMEHGIPSVWAMGVRQDGDPGRPRLVCAAGAHLVPEKAVLSALSELGPLLADLIRRYPDEVHRAQEMVTNPDLVATMHDHSTLYGADAAFDRLSFLTGGTGVRGLPDMEAFTVPGDLDVMRVVDRFLAEGMDVVVIDQTTPEHRARGFSCVKVLVPGTLPMTFGHRNRRVRGLTRPLELPYRLGHRDTPLTPADLNPDPHPFP

SEQ ID NO: 35 - DNA, 2484 bp, nonomuraea sp.
gtgccgccggcccaggccggcggcactccgcccggggtgaggagacagatcatggaggtgggccgcgaaatcgtggtccgcgtggcggggctgcccgccgccgtgctcgccgacctgcgcctgccgcacaccgccgagctcgtcacgcacctgtccgctgagcggcggcggctcgccgccgaggcggctgcgctgtccggcgagctgttcgacctgatcggcgccgccggctccgcccgcgccgccctggtgggcctgcggcgcgcgctcgcccccgggcaccgccccccgtcgccgcgactgatcgggctgtgtcccctgcccgccccgctggccgagcgggtcaccgcctgggtgcgggcccggtacgactgggaggaccggcgccgcgaccttgccggcacgctggacaaggagcgcgccgacgcgctcgaccgcgtacgcgccgcctgcgtcgccccggcgttccggcggggtctggcgctgtccggtggggagctgaccagcacgctggagcgctggctcgccgaccccggccgtgcgccccggcaggggaaggtgctccgcctggtcaagtacctgacccgagctgcggccaagaccagcccgtacggctccttcatggtcagcgcgctggcggggcggcccggcgacgaccctcccatcccggagctgctcaccgtggccgagccgccgggggcgttcctcgacgccgtcggcgacgcgctgctcgccgacccggcactggccggccaggtgccgctgcgccccaaccccagcctgacgtggaccgcccaaggactgcttttcgtccggaccgtccgcgcggcggcgggagagcaggccgggccgaaggaagagatcgccaccgccgggcgcgccgccgcgctggagctgtgcctgcggcacgccgagtcccggcccaccgcgccgcagctggcggagctgctggccgaggcgggggcggatccgggcgaggcggccgcgttcgtcgaccggctggtggcggcgcagctcctgctgccgtgtccgccggtgcacgacgacgatcccgacccgttcggtgcctgggcccgccaggtgtgcgccccggagctgcgcgagctggcggccgcgtcgcggcccgtggccgccgccgtggactcgcccggccagcgccgcgccaggatcgccgaagccgccgccgccgtggcggaccggctcggcatcgacccgcccgccgatccggcgcacgagcacgacgtcagcaccggccgtccggcgccgccaccgctgcccgcgggcgtgctcgccgacctggacgcggtccggcgctggctcgccgtgttcgactggaaggtgcccgtccgggtgggggtcggcgcgttctgccgcgaacgtttcggctcagggtcccgcacgcccttcctggaggcgtgccgggcggcgacggcggcgctgccgcacctgttcggccccgccgcgatgccgtggttcctcgacctgaccggtgacggtcgcctgcgtgagctggaccgtctccggacgcgcgcccgcgatctcgcccgctccggcgtcctcgaccggcggcgggtgctggctgacacggccgactggcccgcctggctgacctggcccgcctccactggtttctacctgcagaccctgcccggtggggtggtgctgaacgcggtgcatgccgggcacggccgcgcggcgggtcgcgtccaccacctcctcgcccgggcgggcgccgcgccgccgcgcccgccccgctccggcctgccgcgtgccgagttcggcggcaggttcggctccgcgctcaacacccgcacgccgagcacgcggtacgagatcgaccaccccggcgcgacgtccggccgcgacccccggcaccgtgtcccgctcggcacgctcatggtggtgcacgacccggacaccgacctggtccacctgcacagcgaccggctcggccgcgtcgaaccggtgcacctcggcatgatgggcgagctggggctgcccgccgtggccggtttcctcgaacgcgccttcgcgcccacctacctgttccacccgagcgtgccgccgttcatctcgctgcgtgacctggccggcgcctcctcggcgcggcgcttccccagggtgagtgtcggcgacgtggtcgtgcagcgcgcccgctggacggtgcccgccggcctggtgcccgcgcgtaccggggcggacggtgaccacctgctcgcgctggccgagtggcggcgcgagcacggcatccccgagcggtgtttcgtgcgcggctggaagccgggcgcagcgctgggcaaggcgcgcaagccgctctacgtcgacttcgcctcctggcatctggtcgccctgttcgaacgggaggcgcgaaccaacgccgccctggtcatcgacgaggccctgccggacccgctggccgacggcgcccccgcgcacgtcaccgagtaccacgtcgagatcggcgaccgggagagagagcaatga

SEQ ID NO: 36 - DNA, 900 bp, nonomuraea sp.
atgaccgaacggcccggctggttcgcggcgcatgtgcactaccgcggcgacctcgacgcgctgctgcgcgaagcggtcgcgccgctggtgcgcgcgcttgccgccgacttcttcttcctgcgctactgggacgggggcagccacctgcggctgcggctgcgcggcggggaccaggtcgcggtcgcccggcacctggacgcctaccttgccgcccaccccgcgccggagaccacctcccaggaggagtacgcccgcgtcgccccggtgctggccgcccgcgagggcatgacgggccacctgacgacgctgcggccgaacaacaccgtggagttcgcggcgtaccggccggagaccgcgaagtacggcaccggcgacgcgctacgcgccgtcgagcggcatttcgtcgagtccagccggtacgcgcttgacgtccttgaccgccgcccgaccggcaaccaacgcgagctcgcagtgctcggcgtcctgctgctcgcctggtacgccgcccgcccactcgatgagccgcctggtgagccggtcgatgacgagctgatcggcgcggtcgaggcgctgtgccggggctggcgcggcggccgggacctgcccgaggagctcgtggcggaggagtacggccgggtgcgggagcgtgtggccgggctggccggcgcgctgcgtgacctcgttccggacccggacgcgcccggcagcagcatgcgcgcgtgggcggcgacgttcgaccggctcgccgccgcgctgcccccgcccgaccggctgcgggtgctcgacacgtgcgcgcatctggcggccaaccggctgggcgtgtccatggcggccgaggtgcggctgcggctgctcgccgcgcgggccctgcgcgaggtcgcgcccgtggggaggcgatga

SEQ ID NO: 37 - DNA, 1002 bp, nonomuraea sp.
atgagctggcgacgcgtcgacgtggcctaccacgatcccgaccttgacgggctgatcctggccacccggccgctgctggccgggacgcccggccgcggctggttccagcggcactgggtccgcggtccgcacctggagctgtggttcgacgccgcgcagccgtcgtgggagcggatccgcgacgtgctggagccctggctgcgcgtcaacccgtcgcgcgcccggatcgaccgagaccggctgctggcgcagcaccggcacctcgccgccgccgaacggatcgacgagccgctgctgccgttctacgccgacaacacgctgcaccgggccgctccgcgctcccgcgcacacgtgctcggtggtccggcggccgaggagctgttccacgacttccacaccaccgccagcgccgtggcttacgacgagctcgacgccgtgcgggcgggagagtcgcggctggtgatggcactggacctgatggtcgccgccgcgcacgcgcacgccgagggcggcgtccgcggcgggttcgtgtcgttccgctcgcacgcggaggcgttcctggcgagcgcgcccggcctgcgcgagcgctgggacgccgagtacgccgcccgggcgggggcgctgcgagcgcggatcaccgcggtggtcgccggaatcccgcgcggccgggcgtgggccgggctgctcgaccggttcgcggaccgcggcgacgagctgatcgcctccggcgcgctgctggtcgagcccgccgggccggacgcggtcgcccggcccgacaccgccttccaccgggcgctgcgcggcaaccggacctggcacgaggaggtgctgcgctcagcaccgttccgccgctaccggctgctgctgaacctgacctacctgcagctttcccggctcggggtgaacgccgtgcagcgggcgctgctgtgccacttcgccgcgtcggcggtcgagcaggagtacggggtgtccgccatcgagatcgccatgggaggcgcgtga

SEQ ID NO: 38 - DNA, 1368 bp, nonomuraea sp.
gtgatgtcagtacaggtcgagcccgcagtgacgcaccggtgggcactgcggcccggcgtctaccgggccaccgcgccaggcggcgatctcatgctcgccgcctggccgcacgccacgccgctcggccgggccacccccgagctgctggccctgctggacggcctggccgccggatcgctgccgctcgacgagcccggcgacctcgcgcccgccgtcgagctgctgcgcgcgggtggctggctgcgcaagacggtggcgtacgcgggacgcgacctctacaccatcacccccgtcgccgcccccaccgacgcgccgccaccgccgcgggacgaaccggcgctgtcgcggttcgcgctgctgcgcaacaccgcggacgggctggtgctggagatgcccggctcctggtgcgacgtccacgtgcacgacccggcggtggccgcgctgctcgccgacccgtccggcgacggcggcctgcccgccgaggccgcggccgcgctgcgcgccgacctggccgccgccggcatgctgatggagcgggaggagccgttcgagcggcggcagtggagcacccacgagctgtggttccatgaacgcagccggctcggcaaccggggctggttcggcgcgcacttcggcggcacgttctgggcccgcggccggcacgacccgccgcccgcgcggccgtccccctaccccggcccggccgtcgagctggcccggccggacctcgcggcgctgcggcgcaccgaccccacgctcaccgccgtgctggaggaccgcgaaagcgtccgcgaccacgacgacgacgatccgatcagcgctgagcagctcggcgagctgctctaccggtgcgcgcgggtgcggctgctgcgtcacctggaaggcttcgagtacgccggcaagccgtaccccgccggtggctcggcgtacgagctagaggtgtacccggtcgtgcggctggcccgcggcctcgaccccggcatgtaccactacgacgcgcacgaccatcggctgcgccaggtccggcccgccggccacccggcggtgcgcaggatgctgaaggtcgccacggagtcgtccgtcgccaaggcgccgccccaggtgctgctggtgatcagcgcgagagtcggccggatcctgtggaagtacgaggcgatggggtacgcgctcatgctgaagcacgtcggcgtgctgcaacagacgctgtacgcggtcgccaccgccatggacctggccccgtgcgcgctcggcagcggggacgatctcgccttcaccgccgccaccggccgggaccggctggtggagtgcgcggtcggcgagttcatggtcggcagcaggcggaaggagcccgcggcatgggagctctga

SEQ ID NO: 39 - DNA, 1827 bp, nonomuraea sp.
atgggagctctgaacctcctcctgcgccccgacgcgcactacgccgaggtcgacggcggcgtctacgtgctgagccaccagggcgagaccttcatctcgggcacctcggtgcaccggtggctcgaccgcctcgcgccgctgctcgacggcacccgcacgctcgaccagctcaccgccggcctgcccgccgaccgggccggcttcgtggccagacttgtcggcacgctcaccgaacgcggcctggtgcgcgaggtcggcccggcggtgtcggacacgctcaccgacgccgaacgcggcgagcaccgtgccctgctggcctacctgggctacttccacgactcgcccggccgcgtcctcgaggacgtgcgcgacaccccgaccgcgatcgtggggtccggtccgctggccgccgcggtggtccgcgcctgcgccgccgccgggctgcgccgggtggagatcggcgaggaggcgggcgcggcccgggtggtcgtccacgtggccgagcgcgccgagcccgaacgggcggcgcggctggaacggcggtgcgccgccgagggggtgctgctcgcacaggtcatgccggggccggacggcatctggtggcagcccgcggcccgcggcggctgctgggcgagcgcgtggcgccgccaccgggcgctcgcgggcgtggatcggacgggcacgccgctcgatccggtggcggtcgcggtggtggccggccaggtggcacacgacgtcttccgcgtcctcgccggactgcgcgaggaggcgccgccccggttggtggtgctcgatccgcggacgctggcgagcaccgcgcacccggtggtggcgcacccgttcgagctgcccgccgcgccgctggacgaggcggcgttcctcgaccgggtcgccgcgctgcgggcagcgcccgcgctgagcgaggcggagttctcccggcgggccaaggggctcatggactcgacagtggggctgttcgccgagatcgacgagggcgacctcgcgcagttgccgctgcacgtcacggcgaccacggtggccgacccgtgcgggctgatcggctccgcgccgcggcccgtggtcaccggcgcggggctcacgttcgaggaggcccgctaccgcgcggcgcgcgccgcgctcgccctggccggcactctcgcgctcgaccggcggaggctggtcgacggccgggtgtgggcgtacgaccttgcggacggggccgcccggctggttccggccgacgaggtcttcgcaagcacggcgggcgggagcacggcgggcgtgccgctcggcgcggcggcagcctactcgtgggccgaggcggtcgcggagggcctggtcgcgcaggccgccgcgctcactctcgccggcgccgggcacgcgggcgagccgtatggacgggcggacctcgccggggcgcctgcgtatctcctcgccatggtgcgggcgctcggcgaggagctgaccgtgcacgacgtcaccgggccgctgggcgtgccggtcgtggtggggacgctgtccggcggggcgaccgcctgcggcgcgggcatgaccacggacgccgccgtcaccgcgtgcctgcgcgacctgctgctgctgcgccaggccgagatcaacgaccagcccgtctacgccccaccatccggcaggcccttgcccgggcatctacgcgggaaccacccggcgcccgccgtgcccggcacggacgccgaccggctggcggcccgcctggccgagcgggggcaccgccctctggcggtgccgctggatcatgaccaggccgtgcacgcggtcctgcccttcgccgtccgggtggtgtgccgatga

SEQ ID NO: 40 - DNA, 1737 bp, nonomuraea sp.
atgatccgcctgcccgaccacggcgagcttgccgtgccgatcggcgacgggccgatgctgccgctgcgcaccgaactcggccgggtggtcatcggcccgctggagcggcccggcgtgcccggatgccgggcctgcgtgcgcatccgcgcggcgcgggtccagcccgacgcccgcaaggccgaggccgtacgcgcccggcacgcccccgcgcccagccagtggctcaccccgctcgccaccgacctggtgcgcgtcctggtcgcgagcgagagcgctgccctggcagccgacaccgacccccgcaccgcgggtgccgtgctggagatcgacctcgccacgctggaaatcaccaggcaccggttcctgcccgacccgctctgcccgcactgcggcggtctgccgccggacggcccggccgagctgaccctgcggccccgccgcaagctcggcggcagtccccggaccaggccggtcgagctgaacgcgctgctggagacgtacgtggacggccgggtcgggatgatccggccgctgaagtcgggcgtgcagggcggactgacggtggccagcgccatgctgccgatccgcgcgggccacggcctggaaccgggggtcggccgtacccgcagctacaccgcgagcaggctggtcgccgtgctggaggcgctggagcgctatggcggcgtcagccccggcggccgccgcaccagcgttaccgccgcctaccgcgacatctccggccacgccgtccatcccgacaccttcggcacacaccccgaggagaactacgacaggcccggcttcgccttccggcgcttcaccgaggacaccgtgtgccggtgggtgtggggctactccttcgccaaggcccagcccgtgctggtgccggagaaccaggtctactactacgccagaaacctgccggacggcgagcagccgttcgtcttcgaggtctccaacggctgcgcgctcggctcgtgcctggaggaggcgatcctgcacggcctgctcgaggtggtcgagcgggacgcgttcctgctcacctggcacgcccggcgggaggttccggtgctgaaccccgcgctggcgagcgacccggtcctgcccatgcaggccgccgcgatcaccgccgagaccgggcaccgggtgctgtgcttcgacaccaccgccgagcacggcatccccagcgtgtgggcgatggccgtggacgtcgaacaccggtcggaccggcccgccaccgcccacgccgccggcgcggcgctcacgctggagcaggccgccatgaacgcgctgagcgagctcggcccgctgctggccgacgtgatccgccgctaccccgaggagcgggaacgggccgaggccatggtccgcgaccccgagcaggtcgccaccatgcacgaccactccctgctctacgcggttcccgccgcggccgaacggctgtcgttcctcaccggccgcgccgacggcccgcggaccggcttcggctcggcccggttcaccggcgacgacctcaccgccgatctgcgggccatgatcgacgcggtgatcgacgccgggatggacgtggtggtcgtcgaccagaccacgccggagcacctggcgggcgggttccgctgcgtgaaggtgctggtgcccggcgcgctgccgatgacgttcggccaccagtaccggcggctggacaacctgccgcggctggcggccgcgcgtaccaccgacccgcacccgttcccatga

SEQ ID NO: 41 - DNA, 2574 bp, nonomuraea sp.
atggcggttattgagcgggaagaaagcgacggcccgccggtcgctgtccgcacgtgcggggtcgcggccgcggagctggaatccctgcgatgggacgagtcctatgcgctggtgacccggctgctcgcggagaccgcccgggtggcggcggacgggcggcggctggccgtggcgctcggtgacgtcatcggagggctcggcccgtccgggtcccggcccgtcctcgtggggctgcggcgggcgctgcacaccgggcgtctcccctccgggcgggagtgggggcccgaggccggggcggcgctgccgtacgggctgaggggcgaggtggaggagtgggtacggcgtgcgcgcgagtgcgccgggctgcgcgcccgcctgcccgaggtcgtcgcccgggagtccctggagaaggaggaacggctgcgcgccgccgcggccgaccccgccttccgcagggggctggcgctggccggcggcgagctggcggtcgacctggagacgtggctcgcggaccccgcgcggcgccccaagccgcagaagctgctcaggctggccaaatacctggtcagggccgccgtgaagaccagcccgtacagcacgttcacgagcaccgggcgggcggtgtggggcggtggcgagcggcacgtggagcgcgtggtgccggtgctggaactcgacggcgtccagacgttctcggacgatcgggtcagggtgaatccgtcggtcaccgtcgcgggcgggacggcggagttcatcgggccgcctcccggggaggcgctggtgtccatcggggtgacggaggcggtggccgcgtgcctgcgcgtcgcggagggcggggagtgggtgccgcggggccggttcgcggaggctctgggggctgagcctgccgcagtcacgaaatttctggacaagttgcttgcggttgggctcctggaggcccggccggacggcgacccgcccccgcacctgctggacgggatccggcgcacggaacgcgccaccgacccgtccaccttccgccacgaactcggccgcctgatcgccgccgcccgcgccgccgcccgcgccaccgcccccggcgctgacacggccaagaacgccgcccctgccgggcgcgccacccgcggcgatggcggtgctgatgccgagcgcgccacccgcggcgatagcgatgctgatgacgggcgcgccacccggggcgacggcggtgctgatgacaggggcgccgcgcggggcgacggcggtgtggggcgggcggttgtgcacgaggtcgcggtctgcgtcgagcccgtggcgaggctggatccggcgcgatggcgtggcgggcttgccgatctcgacgtcgtacgccgctggctggccgttttcgacgccaagcaccccatgcgcctcgcggtcgccgcctacctcaccacccggtacggccccgacccggccgtccccttcctcacgctccaccgccacatccagcgcgagctggccggcaccggcgcggcaggccgggagttgcgcgcgttcctcggctcgtcggccgcctggacccagccgctaggcacctccgccctgcccaggatcagggagctggagcgcctcagagccgaggcgagaagcctggcgctggacgcggaggaccccgacggcacctgccgcgtcacccccggacagctggccgcgcagctggagtcgtggccgtcttggatcgcgatccccgcctcctcggcctgctacgtacaggcggtggccggcaccctggtgctcaacgtcgtccacggcgggcacggccgagccctgcgcaggctcgaccacctcctcgaacgcgccggcggccaccccgagccgccccggatcgaggacccggacggcgcggtctacgccgagttctccggcgacctgggctccacgctcaacgcgcgcccgccgagcacccgatacgagatcgactacccccactcgcccggcacgcgcccgcccgacctgcggctgccgctcacggacctgcacgtgacgctctgcccggacagcggcctgcccgagctgcgttccaagcggctcggccgccgggtcgtgccgttgcatctcgggctggccgccgagttccggcttccgccggccgcgcggttcatcgaacgcgtcttcggccccggctacctgctccatcccagctcgccgccgctggtgcggatgggccgggtcccctcggaggtcacccggtatccacgcgtcgaggccgggcgcgtggtggtgcagcggcgtcgctggctggcgcctgcggccacgctgccggtgcgcgcgaagggcgagggcgacgcggcgtacctcacgcggctgatcgcctgggcggacgagtacggcgtaccgcaccgatcgttcgtccgcgcctggccggagcagaccggcgacaaagggcaggacaaggcgcgcaaaccgctcttcctggacctggcgaacctgttcctggtcaagaacttcgagcggcagatacgtggctgcgcgttcgcgctcttcgaggaggcgctgcccgacccgggaccggagcgggtgaccgaatacctgatcgaggtgggaggccgatga

SEQ ID NO: 42 - DNA, 816 bp, nonomuraea sp.
atgagctgggtgagcgtgcacgtcttctcgcaggggccgctcgacgacatgatcacgggcctggccgggccgctcctcggcgacgacggcttcttcctgcgctactgggagggcggcccgcatctgcggatcagggtcaggtcccacccggcggagcaggtcgtcgagcgggccgaggcgtatctgcgcgagcacccgtcagaggcggtgatcgacccggaggagtacgccaggctcgccgcgaccctcgccgcccgcgaaggcgtccccggctatacgcggcgcctctatccgaacgactccgcccacctcatcccgtaccagccggaacacgaccgctacggcacgggccgctcgctcgacgccgtggagcggcacttccacgactccagcgtgatcgcactccagctgatcggggcggggctcggacacgagcggcggagcatggcgtggctgaccatgctgctggtcgcctggcaggtcgcgggggagggcagggcgttcggcccgcccgctgacgccggggttcaggtgccccacgagcggatggcggcgctggtcgagcacgccagggcggtgacacaggagcctgcgccgagcgagccccgtggcgtggtggccgcctggtacgcctcgatcgcgcggctggtggaggaactggcggcggcggggttcgacgcggcgcggacgggggcgaccgtcgatctggccgcgcatctgctggccaacaggctggggatccgggtgcaggaggaggcccggctgcgtcacctggcgtccggggccacgagggagtcggaggtgggggtcggatga

SEQ ID NO: 43 - DNA, 960 bp, nonomuraea sp.
atgacctggacgcggctgcgggtggactatcacgacggcccgatggacgacctcatcctggacgcgctccgcccggcctggcatgaaatacggggatatttcctgcgccactgggtgtgcggcccgcacctgcggatcttcgtggacggcgacgcgacgccgatcgtccgggcggtcgagcgccacctggccgcctgcccgtccgccggcaccaccgacccgcaggcgctgctccccctgcacgagcgcctggccgagctggaaggcgaacgaggcccgctgctgccatgggcccccgacaacaccgtcaccgccgaacccccggggctcgacaccgagctcgaccggttcctcgcggacttctacgccgacaccaccgaggccgcgttcgacgccctcggccgggtcagagccggcacgccgctgccgggcatcgccttcgacctggtggtcgccaccgcgcacgacctgtcggagggcggcctgcccactgccaggacctcgctgcgctcgcacgccgaggcttacctgtcgcgcctgccgggaggcgtccgcgccaagtggcaggcgcactacgaacgcaaccaggagccgctgaccgccaggatcaaggccctgaccggcgccggagagcccggcgcgtggctgcgtacgatccgggccacccgcgaccggggccgtaccctcatcgacgagggacggctgagcctcggctacgccaccgacggcccttccacgcggcccccgctcgccgcggtctcccccttccaccggaacctggagaccgacgaacgctggctcgccctcaaggacacccccgccttcgccgcctaccgcctggccctgaactgcgcgtacctccacctcacccggctcggcctcacgcccgaccagcgcttcctgatctgccacctcgccgcgagcgccgccgacgacgtctaccgggccgtggcctcatga

SEQ ID NO: 44 - DNA, 1287 bp, nonomuraea sp.
atgaccggggaacggtacgagctgcgcgccggcgtgcacctcgcccggctggacgcgggggtccgcctgttcgcctggccgcacgccgagaccctcggcgagctcacgcgggatgagctcgacctgctccgcgacctggccgccggaccccgcaccgccgggggaccgctcgtcgaacgcctgcgcgccggaggctggctcaagatcacgattacccgggacggcgtgcccctctacacgctcgccccgctgcgcccgccgccgccacggccggcggtgccggagcggccggttctgtccaggttcgccacgctgcggcgcgagggggacgcgctggtcgccggctccccgctggcctggtgcgacctgtacgtccacgaccccggcctggtcggcgacgtggtccggcccggcggcgaggtcctggccgacctggtgtgggccgggctggccgtgcccgaggaggccgaggaccgggagctgcggttccggcagtgggcgccgcacgagctggagttccatgaccggagccgggtggggcaccgcggctacctgggcgatgggttcggcgggacgttctgggcccgcggccggttcgacccgctgcccgcccggccccggccctaccccggccgggcgatcgagctgcccacgcccgacctggacgaactgcggctcaaggacccctcgctcacggacgtgctggagggccgcaggtccgtacgcgagcacgacgacgagcgcccgatcaccctcgacctgctcggcgagctgctgtaccgcacggccagggtgcgggagtgctggacggccgacggcgtcgaatacacccgcaagccgtacccctccggagggtccgtctacgagctggagatctaccccgtcgtcaggaacgtggccgggctcgaacccggcatgtaccactacgacgggcacgaccacgtgctcaggccggtacgtgaggccggccatcccgccgtacgccggctgctgaccgtcgccgggcacggcgccgccaccggccgccgcccccaggtcctgctcgcggtctccagccgcgccggccggctgctgtggaagtacgaaggcatggggtacgcgctgacgctcaagcacgtgggtgtgctctaccagacgctctactgcgtggccacggcgatgggcctggcggcctgcgggctcggcagcggcgactcggccgcgttcgccgaggcgaccgggcgggacccgttggaggagtgcgcggtcggcgaattcatgatcggaaccgcggaggcaccacgttga

SEQ ID NO: 45 - DNA, 1893 bp, nonomuraea sp.
ttgaacgacttcaaacccagactccccgcggacatacgcttcgcgcccacctccggcggtgtcgccttcctcacccgcgaaggcgtcgtgcggatgaacggggcctccgtccacctgtgggtggagcggctggccgtgcacctcgacgggagcaggaccgtggccgagctgaccgcggagctgcccgacgagcggcgggcgttcgtgacgagcctgctgaccacgctgcgcgagcgcgggctcgtggaggacgcgggagagcgcggcgtgctgcccgtggcggccggcaccgcgtgctcgctgctggccgacgcggaccggttcggcgagctggcgcccgccctggccgacgccctgcgcaggctggggctggaggtgctgacagagccgtcgggcggggcgctcgtgcacgtcgcggcggccgacgcggacgggctggccgccgccgggcgcctcgaccggctgagcgccgagcgcgggctgcccatcgcgcacgtgctgctgcgcgagggcgaggcgtggtggagcgagaccggcgtcgtcgggcgcgacgtcccgccgtgggcggccgggtggcggcggctggcggccatggaggccgtcccgcgtaccgccgggcacgtctccgtgtcgcccgtcgccgcggccgtggtcgccgggcaggtggtggcggacctggggcgctgcctgtccggaacgccccgcgtgtccggcggtccgcggctgcacgtggtggacctggacgggttcgggatgcggacgcactccttcctgccgcacctgtcggcgctgccgcccggcacgggggtgagcgcggggccgctcggcgaggaggagttctcgcggcgggcggccaggttgatggactcgcggacgggggtgttcgccgagatcgaggagggggagtacgggcagctcccgctgcacgtgtcggtgaccacggtggccgatccgatgggggccctcgggggtgcgctggcgcgggtgatcggcgtgggggcggacttcgccaccgcgcgataccgggcggcgcttaaggcactgaccgtgcacgggctgctcgccctcgacccccgccgcctggtcacggccgaggggacgcccctgaccggcgcgggggaacccgccagcggcttcccggctgatccgggcgcgagggaacggccgatcggtggcttttcggctgatccgggtgcgggtgagcggccggacgacggtctcctggacgacccgctggtgggcgacgtcgcggcgggacgggtcgaggcgttcgtgcgcggccgtgacctcgtcgagggcggtgaggtgctgctgcccgcccgcgcggtgttcctggatccgcacgccaccggggccgccgcggggtactcggccgaggaggcgctggtcgcggggttactggaccactgtgcggcactggccgggctcgacgggacgctgccgcccgtggacgtgggcggcgcgcgggcggccgacgacgagaccgcgcggtcggcggcgctgctggacgcgatcgggctgccgtaccaggtgctggacgccaccggcccgtcgggcgtgcccgtgcacgcggggacgctcggcgggcggctggtggcgcgggcggcgggcgtcacgcccctggccgcgctgcgggccacgctcgaagggatcctggccgcgtaccagggcattccgggccccgcacccctgcccgacctgtgcccggccgtcgcgcaggagccgccggtcgtcctcgacacgggggcggccgtgctcggcgccggtgagctcgccgccgccctggccggcgccgggctgcgggtcagcgccgttccgctggatcacgaccgggaggtacacgcgctcatgccgcacgtcgtccgcgtcgtcacggagccgctccatggctga

SEQ ID NO: 46 - DNA, 1833 bp, nonomuraea sp.
gtggggacgggcctgctggccgaggagatcacgatcgcgctgaaggaccgcgacgggcgggcagaggtgacgatcaccgcctgcgacggctgggcgcgcggcccccggcgcgacggggcggcgtggttgcccgtgcgggtcgagtacggggtcgccgtcatcgggccgctggaacgccccggcgaacccggctgcgtgacctgcttcgagacccgccgggaccgggtcaggaccgacagcgccgatcgggccgccgtcctggccgtccacggccccgaactcgccgggcgggcctccccgtacctcggccgcctggccgcccgcacgatcgcggccctggccgcgcacgccgccgagggccccggatgcgccgtctggtacgtcgacctgcgcacgctcaccgccgaacgccacacgttcctgccggaaccccactgccccgactgcggcgacctgccggacgacgaccgcgcgtccgccgccttcgagcccggcgccgccccgcgacctcccggctaccgggtcaggaacgtgccggccgagcacggcgcgctgctcgacacgtacgtggacggcgaatgcgggctgatcaggcccctcgtccgggacacgctcggcggcctggtgatcgccggggcgatgctgccgctgcggttcgagggcggttcggagcccggcgtgggccgcacccgcggctaccgggccagcgaggtcaccgccgtgctcgaagcgctcgaacggtggggcggcgtcgagcccggcggcaagcgcacccgcgtgcgggcctcgtacgcggaggtggccgcggacgcgctcgaccctcgaacgctgggcgagcacgacacctcctcctacgagatggaggggttcgcgttccggccgttcgccgaggacgccgtctgcgactgggtgtgggggtactccttcgcccggcgctcgccgatcctcgtgccggagagcctggcctattactacgtacgggggcgggagcggccgttcctcttcgagatctcgaacgggtgcgcgctcggcggcagcatggcggaggccctgctctacgggctgctggagacggtcgagcgggacgcctttctcatgacctggtacgggcggctcccggtgccgcggatcgaccccggcacggcacgggaccgcaccatcccgctccaggccgccgcgatcatcgcggagaccggttatcgggtggagctgtacgacacgacgatggagcacggcatccccagcgtgtgggcgatgggcgtgcggcaggacggcgatccgggcaggccgcgcctggtgtgcgccgccggggcccacctggtgccggagaaggccgtgctcagcgcgctgagcgagctggggccgctgctggccgacctgatccggcggtatccggacgaggtgcaccgagcccaggagatggtgacaaatcctgatctggtggcgacgatgcacgaccactccaccctgtacggcgccgacgcggccttcgaccggctctcgttcctgaccggcggcacgggcgtgcgggggctgcccgacatggaggcgttcaccgtgcccggtgatctcgacgtcatgagggtggtggaccgcttcctcgccgaggggatggacgtcgtcgtcatcgaccagaccacgccggagcatcgggcccgcggtttctcctgcgtcaaggtgctggtgcccggcacgctcccgatgaccttcggccaccgcaaccgcagggtgcggggcctgacccgcccgctggagctgccgtaccggctcggccaccgcgacacgcccctgacccccgccgacctgaaccccgacccacaccccttcccatga

SEQ ID NO: 47 - protein, 237 aa, nonomuraea sp.
VTARRRDVERVDRIVDAAAELLPERGYRRLTVEEVASRAGVSKSSVYLHWNTKDEIFYAVLDREFTVLAQAAVDRVRRDPAEVLAHRTAANLFQIVACRPLLQALLVDDRTTLGSLQPAKSAVFRASLANLNELMYRYLHALRINGLLCPEVDPRIMRKAISEMLRGMTFSAGAKPLAETRLAKLSQVVAVTVQRAFEPGDVPELDRINAAAAEVFKAFDELMPAEETMRAERPVAL

SEQ ID NO: 48 - protein, 633 aa, nonomuraea sp.
VTVVRAHVRQEPAALVVGRGLLADRLAAVTGRLDPGVVLAVSDAPDPAVLAEARATGRPVLPVLAEPGRVRVGPLERPGEPGCSDCLSLRRHRAASRSAERAAAWLRHGPDLAATPSPLLAPVALDLVAELVTALVTAPAPAHRGILLADLSDLSVTPHAFLPDPFCPRCGTLPDDDPARARITLVPRPKPSPDRHRVWDAEGRLDRLRQIYVDDHCGLVHSLNPAALGSLAVAGAPIRLRGTTAFEPGFGRSRSYRRSSVIALLEALERYGAIGPGGRRGTVRASYASLGNLAVDPRSLGLHPPEHYALPGFPYRPFTPEAVCRWLWGHSFATGGPVLVPERNVHYGPSDDDRPFCYELANGCALGSCLEEAVFHGILEVLERDAFLLTWYTRARAPRLDLGTARDPTIPLVAAAITAETGYLVECYDITPDHGVPCVWALARHPSDGEPATISAAAAGPGLEPAAAGALAELGPMVPTVRDHFPQHAGRARELAADGRRVRSMIDHYLVYGVRSAARRLSFLTDGTEEVPFAPAPDGFRHDDLTADLRFLLDRLADSGLDVVVVDLTTPEHRAGGLRCVKVLVPGTVPMTFGEQNRRTWGLPRLLDPAVVRGRGMPVRGHADLNPDPHPFP

SEQ ID NO: 49 - protein, 829 aa, nonomuraea sp.
VSRHAPPAVARVGGLPAAALHGVACPEATRLAGRVVHLTEELTRRAAVLSDALYEVIGAAGAHKPILVAIRRDLHGLRRPKRVEVLPAPLAEPVRTWISLWEERARVHAVLPDVLAREERLAWEALRELAATPAVRHGLAHASPDLSADLEKWLADTGWRPRPSTLASLLRYVKRVAAKTSPFSTFTTVHEVRWVDGGAAWQVPDTAPTVVVEADVGLRLLVESVLPRMPEVAAARVVRLSPAAYASGDRLAFPEPGGRMRAVDRTSALDTLVELLRAEQRWDAAIAKLTGAGLAGHGAEDGTRDSASDGTAAAEQVLSGLVRGGLVEAVVPVPGQAARPFARLADWAAPAAPAGPLHRIQVALDQAGPLGDGDPLASACAHVARRLTAELPALGLPVMPVPDLRRRVLRESALGAPVACALREWRPVLADLQRVRRWLAVHDPMLPLRLTLADRVRDWFGPGSAAPLLDVYARVRTAEPGTPLDPDFLEHPDPLAGVTDPRLTRLRELRAASVAALTGGRAEEALSEPPAWVRDPGPVTCYVQPFQGEDGLRLVLNAAHGGHGRGITRWTRLLGAEPPPGEPPAAESPPGESPPGELPPGACLVAELPGTFGHSLNLHAPATGWELTYPGAVNQAPPERRVPLAELQVRHDAGRGVVELWWPRAGRRVVPVHAGMMSETLLPPLARLLVEAFGTTHLTHPTLPPVVRAAGPRIDLGRVTLARAQWTARREEIPRRGGDDAAHLVAVHAWLRAAGIPRRCFVRVREPQVRRDRLAFDKRHKPVFVDFGSWPSVLEFDRIVTRATGDLELTEALPDGERAVELAIEIGAR

SEQ ID NO: 50 - protein, 344 aa, nonomuraea sp.
VTRYPLPRPHPLGIHPGYARLREGCPVARVDSPYGPAWLITRYADVAAVLTDARYSRAAATEDDGGILLNTDPPEHDRLRRLVVAHTGAAAVERLRPRAEEIATTLAKRIPAEAEFVSAFAEPYSHRVLALFVGHLVGLPAGELSPMATVLTLARVPDRERAAAFAELRRRLRSQVDDETLAVVLNIVFGGHAAVVAALGYCLLAALAEPLPSLTGDVRRIAELVEETLRLAPPGDRTLLRRTLEPVELGGRELPAGALVIPSIAAAGRDPDRPTGGPAGRHLAFGRGPHACLGMALARMELRVALQALAEHVPGLRPAVSPATLRRTCEELAVSPLAGIPIRV

SEQ ID NO: 51 - protein, 228 aa, nonomuraea sp.
MVHAFGVRNRGRIVSAEHHYRDRLRALSAYLRKVDNEPEAAAELRADPEKALREAGVDQAFDRPEAFRAFVGKLAVLSGEAWLATVHSMIELCEIGADPQPPAGPNISFRISSDGGVTAIANRGEIAKKVQPNPFHAGSAAASPGGRLRIYPGYATSELSARLGERYLSTFYQRTLLKRVVLDPGTVVEDADAGEGITVNRSQYRGVGFELHTRADGADREIVAALVR

SEQ ID NO: 52 - protein, 372 aa, nonomuraea sp.
MLSSALEVDIDEAAVAADLRELAAALDRSGYGEILTCFLPHKAQAHIWAQTAAKIDGPLRTLMELFLLGRAVPQDDLPARVGDLIPGLAATGLVQTGQGAVWLPNLILLRPMGQWLWCQRPHPSPTMYFGDDSLALVHRMVTHRGGHTLDLCAGPGVQALTASLRSDRVTAVEINPVAAALCRTNIAMNGLGDRMEVRLGDLYGAVPGEVFDDIVSNPPLLPVPEDVQFAFVGDGGRDGFDISWTILDGLPEHLSDRGACRIVGCVLSDGYVPVVMDGLGKWAVEHDFDVLLTVTAHVEAHRDSSFLRSMSLMSSAISGEPADVLQERYAADYAELGGSHVAFYELCARRGGGTARLADVSATKRSAEVWFV

SEQ ID NO: 53 - protein, 122 aa, nonomuraea sp.
VYVVIVAFDLKESSVDFAELRAWVRDRAADDYSRLPGMRFKTWFSDERKRLWGAVYLVESMLSFDRDNLPLLPDGRTGPVGTRPTSVMVLELEAFVTGPDGLDGIEALARQGLSMTGGGHDH

SEQ ID NO: 54 - protein, 416 aa, nonomuraea sp.
MTTEATTAVTTRANAGALSLPARGTDRPLLLYVNIPFCNSKCHFCDWVVDVPVSDLRLAPAAPRRVDYLAALCTQIRTHAPALRAAGYRGEVMYWGGGTATVLTAAEIEQVHACLAAEFDLTALAEATIEGSPESVDPAKLRLLRDLGFDRVSLGVQSFDETRLRRIGRAHSAGQAVRSVEDAHAAGFDNINIDLIVGFPDQSLAEVEQTVRRALDLPVNHFSVYSYRATEGTVMRRQIERSGTAIELEHQLQSYRLAGEMLAAAGHPEYAVSYFGAPRCLADEAYYRLSMDWIGFGTGANSLINQRYLLNGRGRMRDFTSRPGEFEVDLPAGVPSLTVQWLPRALGTAEGIDAATFQRRTGTSLRAACEEPELNAFLTRVNEFGRLVVDRDGIRLADENRSDVLSRTFAAMGWVS

SEQ ID NO: 55 - protein, 339 aa, nonomuraea sp.
VEAMRTLLAAADLVTPMALRVSATLRLADHLAAGTTGAAELAAAAGVDEAALTRLLRYLVARGVFTEPAPGEFGLNPAAELLRDGRPDRLRDWLDLTGPIGRADLAFGSLLDVVRTGKPGYPMIHGRGFWDDLAAEPALATAYDTLMGGKRQWAATTLAALDWSRSRHVVDVGGGNGTLLSCLLAAHPHLRGTVVDRPTSAKAAGAVLASAGVADRGEFRAGDFFEPLPVHGADTYLLSSILHDWDDAAATAILRRCAEAAAPGGRVLLCELVAMAGPDRRTVTHMDLCMLVYFGGRERTAADFAALAGAAGLEPRSVTPLPPHDWGNALVEYAVPAAR

SEQ ID NO: 56 - protein, 204 aa, nonomuraea sp.
MAKVISTLFISADGVAEIDPDWHFPYFDENMGRAVGEDYDTADVLLIGRETFDSFAGAWPDREAAGGDDAPFAKQLGDMRKVVVSRQPLEFSWRNCELITGDLLDAVTSLKADAGIRGILIPGSISVVQQLLAAGLVDELRLLVHPVAARKGRRLFDDGDAPYHLSVTATETFPTGVIRVIYSPTEAPTKVGYDEVEAQVSTGE

SEQ ID NO: 57 - protein, 403 aa, nonomuraea sp.
MTKPLLIYANVPFCNSKCHFCDWVVQVPVRDLRLGEQSPGRVAYLEAIRTQIRVQAPALREHYHPDIVYWGGGTASILGPHEIESLYGTLSAEFDLSTVREATIEGSPESLDRDKLRLLRELGFNRISIGVQSFDDARLRRLGRAHAADQALTAVQDAHEAGFTNINIDLIVGFPGQTGQEVADSVRTALTLPINHFSIYPYRASPGTVLRKQVHRGAQLDLNLQLQAYGIARELLEAAGFPEYAMSYFGHPRCQSDEAYYQLRMDWIGFGSGANSLIGRRYLSYEKGKLAHYNTNPLAFDINAPAHSPQLTLHFLSQALTTAEGLDARLYQQRTGVPLRTACSHPEVMTYLERMNNHGRLIADHNGIRLHRDDIAQTFIALNWIPTPDTTPNEVIPLTPSPT

SEQ ID NO: 58 - protein, 387 aa, nonomuraea sp.
MTAPVRPPSVTDGGTSLFAWLREMRDTHPVWRDSYGMYHVFRYDDVRAVLADHERFSSDRTRLMGRQPFGQGGITMIDPPEHRHQRRLITAAFTPSSVAALEPRIAAIADELLDALPGPDFDLVESLAYPLPVTVVAELLGVPPSDRDLFRTWSDRLMSLQVPDFADPSLAGRVAAAMAEMNDYLREHCADRRTRPRDDLLTRLVHAEIDGERLDAEQVVNTASLLLLAGHVTTTVLIGNTVLCLADAPEAAHRTRADMSLIPQALEESMRLRSPFMQAGRVTTQDVQVAGETIPANRFVMAWLLSANHDERHFADPERFDLDRQLTGQLAFGHGVHFCLGAQLGRLEGRIALERLLSRFTELRPEKRSFYESQIFGVREMHVRGSS

SEQ ID NO: 59 - protein, 406 aa, nonomuraea sp.
VNETHVPPDPTMTMPIDSRPRIKDGGQTLLKWLGTMRDEQPVWRDAFGIFHVFRHAAVQRVMSDYAAFSSDINRLRPGGDPFSAGSLMLTDPPEHRKLRKLISQAFTPKMSSDMVPRIAELTTALLDEIDEDEFDLVEKIAHPLPVMVIAELLGIPIHDRELFRTWADRLVALHVEDPRDIEIGRMVGQAMREMGEYVLTHVRERRTRPQDDLVSRLVAAEVDGDRLTDAEIVNSACLLLLAGQITSTMALGNAFLCFRDAPGVESAVRADRELIAPAFEEVLRLRPPLTQAARLPTADVEIDGTPIPAGSMVINWLLSANYDERQFPDPYRLDPGRQPNRQYAFGHGIHFCLGAPLARVEGKVALELVFDRFEQVEIDPDAELSYYEDPMFGVKSLPVRVKRAPR

SEQ ID NO: 60 - protein, 243 aa, nonomuraea sp.
MSTVSTRSTGARTSDDRVRRTLDVTRSLLLRSGYKRTTMDEIARRADIGKGTIYLSWDTKDDLIRTLVIQEIVGVCQDISRIAVLRPAVARLSEFSRELFTLVFKYPLFRALYTYDKETLGRACDDPQLGFQCYRFTTFTPFRDYLRMLHESGMWDPSHGFALDALLSGFIKLHLHAEIAGAKPDLAAHADSLAGLIRSSFEPADQVPAEELTDPARRTVEIFDGAAAKYRAKLIPQPLAASV

SEQ ID NO: 61 - protein, 444 aa, nonomuraea sp.
MTTEPFPAPTGPPAAPTGPSPALAEPSAASPGPLSVRQGPSPVTAELRDLAIFRADLHAHPELSWQEARTADRLAGRLAAAGYEVTTGMGGHGVVGRLCRGDGVTVMLRAELDALPVKEETGLSYASTATATTLDGRTVPVSHACGHDLHLACLVGAARRLAACDDWRGTVLVIGQPAEETLEGAAAMLADGLYERFGVPDVALAQHVSPFPAGLIAYPEPPTAAGAELRVVVTGDGGHVGDIGRAGHVDRAVGRNPVAAVAALVHRLDQTAFDQAIVTVGTLHAGERANVIPTLAEAGITVRAATEEAVTRAVARVARLAEETAGAGVIVVSRVPPGVNDPAATALVRRAHEAALGAVVTAPGGSACEDFPLYGVPSVYWYVGAAPPAGLVGRPHTGTFRPDPVPTLRAGVTAMQTAALAVLANASQFAPPSRYHGPGAVAEH

SEQ ID NO: 62 - DNA, 22848 bp, nonomuraea sp.
gtgacggcgcggcgacgcgacgtcgaacgggtggatcggatcgttgacgcggcagcggaactgctgcctgagcggggatatcggcgactgacggtcgaggaggtcgcaagccgggcgggcgtctccaaaagcagcgtctacctgcactggaacaccaaggacgaaatcttctacgccgtgctggaccgcgagttcaccgtgctggcgcaggcggccgtggaccgggtcaggcgcgatccagccgaggtgctggcgcaccggacggcggccaacctcttccagatcgtcgcctgccggccactgctacaggcgttactcgtcgacgaccggacaacgctcggatcgctgcaacccgcgaaatctgctgttttccgggccagcctcgcgaatctcaacgaactgatgtatcgctatcttcacgcgctacgcataaacggacttctctgccccgaggtcgacccgcgtattatgcggaaagcgatttctgaaatgctgcgcggaatgacgttctccgcgggcgcgaaaccgttggccgagacacggctggccaagttgtcccaggtcgtcgcggtgaccgtgcagcgcgccttcgaaccgggcgacgtgcccgaactcgaccggatcaacgccgccgcggcggaggtgttcaaggcgttcgacgagctcatgccggccgaggagacgatgcgcgccgaacgcccggtggcgctgtgaccgtggtgcgcgcgcacgtccgccaggagccggccgcgctcgtcgtcggtcgcggcctgctcgccgaccggctcgcggcggtgaccggccggctagaccccggagtggtgctcgcggtgtcggacgcgccggaccccgccgtgctcgcggaggccagggcgacgggccggcccgtgctgcccgtgctcgccgagccgggccgggtacgggtcgggccgctggaacggccgggcgagccgggctgctccgactgcctgagcctgcgcaggcaccgcgccgccagccggtcggccgaacgggccgccgcctggctccggcacggcccggacctggccgccacgccctcgccgctgctcgcgccggtggcgctcgacctggtggccgagctggtcaccgcgctggtcaccgcccccgcgccggcccaccgtggcatcctgctggccgacctgtccgacctgagcgtgacaccgcacgccttcctgcccgatccgttctgcccgcgctgcggcaccctgcccgacgacgacccggcgcgggcgcggatcacgctcgtgccgcgcccgaaaccgtcgccggaccggcaccgggtgtgggacgccgaaggccggctcgaccggctcaggcagatctacgtcgacgaccactgcggcctggtccactcgctcaacccggccgcgctgggctcgctcgccgtcgccggtgcgccgatccggctgcgcggcaccaccgccttcgaacccggattcggccgctcccgcagctaccgccggagctccgtcatcgcgctgctcgaagccctggaacggtacggcgcgatcggtcccggtggcaggcgcggcaccgtgcgcgccagctacgcctccctcgggaacctggcggtggatcccaggtcgctggggttgcacccgcccgagcactacgcgctgccgggcttcccctaccggccgttcaccccggaagcggtgtgccgctggctgtgggggcactcgttcgccaccggcggcccggtgctggtgcccgagcgcaacgtccactacggcccgtccgacgacgatcggccgttctgctacgagctcgccaatggctgcgctctcggctcgtgcctggaggaggccgtcttccacggcattctggaggtgctggaacgcgacgcgttcctgctgacctggtacacccgcgcccgcgcgccgcggctcgacctcggcaccgcccgcgaccccacgatcccgctggtcgccgccgcgatcacggcggagaccggctatctcgtcgagtgctacgacatcacgcccgaccacggggtgccctgcgtgtgggcgctggcccgccatccttccgacggcgagccggccacgatcagcgccgcggcggccggccccggcctggaacccgccgccgccggcgcgctggccgagctcggaccgatggtgccgaccgtgcgcgaccacttcccacagcacgccggccgggcgcgggagctggccgcggacggccggcgggtgcgatccatgatcgaccactatctcgtgtacggcgtgcggtcggcggcacgccgcttgtcgttcctcaccgacggcacggaggaggtgcccttcgcgcccgcaccggacggcttccggcacgacgacctcaccgccgacctgaggttcctcctcgaccggctggcggacagcgggctcgacgtggtcgtggtggacctgaccacgccggagcaccgcgcgggcgggcttcgctgcgtgaaggtgctggtgcccggtacggtgccgatgacgttcggcgagcagaaccgccgcacgtggggcctgccgaggctgctcgacccggccgtggtgcgggggcgcggcatgccggtacgcggccacgctgatctgaaccccgacccccaccccttcccgtgagccgccacgcgccgcccgccgtcgcccgggtgggcgggctgccggccgccgcgctgcacggggtggcctgccccgaggcgacccggctcgccggccgcgtggtccacctcaccgaggagctgacccgacgcgccgcggtgctgtccgacgctctctacgaggtgatcggcgccgccggcgcgcacaagccgatcctggtggcgatccgccgcgacctgcacgggctgcgccggcccaaacgcgtcgaggtgctgcccgcgccgttggccgagccggtccggacgtggatctcgctgtgggaagagcgcgcccgcgtccacgccgtgctgccggacgtgctcgcccgcgaggagcgcttggcctgggaggcgctgcgggagctcgccgcgaccccggcggtgcggcacgggctcgcgcacgccagcccggacctctccgccgacctggagaaatggctcgccgatacggggtggcggccgcgaccgagcacgctggcgagcctgctgcggtacgtgaagcgcgtggcggccaagacgagccccttcagcacgttcaccaccgtccacgaggtccgctgggtggacggcggcgcggcctggcaggtgccggacacggcgccgaccgtggtggtggaggccgacgtggggctccggctgctggtcgagtccgtgctgccgcgcatgcccgaggtggccgccgcgcgggtggtgcggctgtcgccggccgcgtacgcgtcgggggaccggctggcgttcccggaaccgggcgggcggatgcgcgcggtggaccggacctccgcactggacaccctggtcgagctgctgcgcgccgagcagcggtgggacgcggcgatcgccaagctcaccggtgccggcctggcaggccacggagcagaggacggtacgcgtgacagcgcgagcgacggaacggcggcggccgagcaggtgctgagcgggctggtgcgcggcgggctggtggaggccgtcgtgccggtgcccggccaggcggcccgtccgttcgcgcgcctcgccgactgggcggcgcccgctgcgcccgccggcccgctgcaccgtatccaggtggccttggaccaggccgggccgctcggcgacggcgacccgttggcgtcggcctgcgcccacgtggcccgccgcctcaccgccgaactccccgccctgggcctgcccgtcatgccggtgcccgacctgcgccgtcgtgtgctgcgcgagtcggcgctcggcgcgccggtcgcctgcgcgctgcgggagtggcgacccgtgctggccgacctccaacgggtccgccgctggctcgccgtgcacgacccgatgctgccgcttcgcctcaccctggccgaccgggtacgcgactggttcggccccggctcggccgcgcccctgctggacgtgtacgccagggtccggaccgccgaaccgggcacgcccctggatcccgacttcctcgaacaccccgacccgctcgccggcgtcaccgacccgcgcctgacccgcctgcgcgagctgcgcgccgcgtcggtcgccgcgctcaccggggggcgggcggaggaggcgctgagtgagccgccggcctgggtacgcgatccggggccggtcacctgctatgtccagccgttccagggggaggacggcctgcggctggtcctcaacgccgcccacggcggtcacggccggggcatcacccgctggaccaggctgctgggcgccgagccgccgcccggcgagcccccggctgccgagtccccgcccggtgagtccccgcccggtgagctcccgcccggtgcgtgcctggtcgccgagctgccgggcacgttcggccacagcctcaacctgcacgcgcccgccaccggctgggagctgacctacccgggcgcggtgaaccaggccccgccggagcgccgcgtcccgctggccgagctccaggtccggcacgacgccgggcgaggcgtcgtcgagctgtggtggccgcgcgccggccggcgcgtcgtgccggtgcacgccggcatgatgtcggagacgctgctgccgcccctggcccgcctgctcgtcgaggcgttcggcaccacccacctcacccatccgaccctgccgcccgtggtgcgggccgccggtccgcgcatcgacctgggccgcgtcacgctcgccagggcccagtggacggcccgccgcgaggagattccgcggcggggcggcgacgacgccgcccacctcgtcgccgtccacgcctggctgcgcgccgccgggatcccgcgccgctgcttcgtccgcgtccgcgaaccgcaggtgcggagggaccggctcgccttcgacaagcggcacaagccggtgttcgtcgacttcgggagctggccgtcggtgctggagttcgaccggatcgtcacccgggccaccggggacctggaactgaccgaggcactgcccgacggcgagcgggcggtggagctggcgatcgagataggagcccggtgacgcggtacccgctgccccgcccccaccccctcggcatccaccccggctacgcccggctgcgcgagggctgccccgtcgcgcgggtcgactcgccgtacggtccggcctggctgatcacccggtacgccgatgtggcggcggtactcaccgacgcccggtacagccgcgcggcggcgacggaggacgacggcggcatcctgctcaacaccgacccgcccgagcacgaccggctgcgcaggctcgtggtggcgcacaccggcgccgccgccgtcgagcggctccggccgcgggccgaggagatcgccaccaccctggcgaaacgcatcccggcggaggccgagttcgtaagcgcgttcgccgagccgtactcacatcgggtgctcgcgctgttcgtcgggcacctggtcggcctgccggccggcgagctgagcccgatggccaccgtgctcacgctggcgcgggtgcccgaccgggagcgggcggcggcgttcgccgaactgcgccggcggctgcggagccaggtggacgacgagacgctcgccgtggtgctcaacatcgtcttcggcggccacgcggcggtggtggccgcgctgggctactgcctgctcgcggcgctggccgagccgctgccgagcctgaccggcgatgtccggcggatcgccgagctggtcgaggagacgctgcgcctggctccgcccggcgaccggaccctgctgcgccgcaccctcgagcccgtcgagctgggcgggcgggagctgcccgcgggagcgctggtgatcccgtcgatcgcggcggccggccgggatccggaccggcccaccggcgggccggcgggccggcacctggcgttcggccgcggcccgcacgcgtgcctcggcatggcgctggcccggatggagctccgggtcgcgctccaggcgctggccgagcacgtccccggactgcggccggcggtcagcccggccacgctcaggcgtacgtgcgaggaattggcggtcagcccgcttgcgggaatcccgattcgggtgtagtgggcagaaaaccgaataaacgaatttggtcagcgggtgcccattccttgccgcggatcacgcccgggtcatactctcggttccagaaatggtgcacgcattcggtgtgagaaaccgagggagaatcgtgtccgcagaacaccattatcgtgacaggctacgcgccctgtccgcatatctacgcaaggtcgacaatgagccggaggccgccgccgagctgcgcgctgacccggaaaaggccctccgggaggccggcgtcgaccaggcgttcgaccgccctgaagcgttccgcgcatttgtcggaaaactggcggtattaagcggagaagcatggctcgcgaccgttcactcgatgattgaactgtgtgagatcggtgcggatccgcagcctcctgctgggcccaacatctcattccgcatatcttccgacggcggggtgacggccatcgccaatcgcggtgagatcgccaagaaggtgcagcccaacccctttcacgccggctccgccgccgcgtcgccgggcggccggctgcgcatctatcccggctacgccaccagcgaactgtcggcccgcctcggtgagcgctacctgtccacgttctaccagcgcaccctgctcaagcgcgtcgtgctcgacccgggcaccgtggtcgaggacgccgacgccggtgagggcatcaccgtcaaccggtcacagtaccgaggcgtcgggttcgaactgcacaccagggccgacggcgcggaccgcgagatcgtcgccgcgctcgtgcgctgacccccggcacgagatcacgacatcacaccccacggaggcgggatgctgagcagcgcgctcgaagtagacatcgacgaggcggccgtcgccgccgacctcagggaactggccgcggcgctcgaccgcagcggctacggcgagatcctgacgtgcttcctgccgcacaaggcgcaggcgcacatctgggcgcagaccgccgcgaagatcgacggtccgctacggacactgatggagctcttcctgctcggccgggcagtgccgcaggacgacctgccggcccgggtcggcgacctcatccccgggctcgccgccacgggcctggtgcagaccgggcagggcgcggtgtggctgcccaaccttatcctgctgcggccgatgggccagtggctgtggtgtcagcgcccgcacccgtcgcccacgatgtacttcggcgacgactcgctcgcgctggtgcaccgcatggtgacccatcgcggcggccacaccctcgacctgtgcgcggggccgggcgtgcaggcgctgaccgcctcgctgcgcagcgaccgcgtcaccgccgtcgagatcaaccccgtcgccgccgcgctgtgccgcaccaacatcgccatgaacggcctgggcgaccggatggaggtccgcctcggcgacctctacggcgcggtgccgggcgaggtcttcgacgacatcgtgtccaacccgccgctgctgccggtgcccgaggacgtccagttcgccttcgtcggcgacggtggccgcgacggcttcgacatctcctggaccatcctcgacggcctgcccgaacacctgtccgatcgcggtgcgtgccggatcgtcggctgcgtgctcagcgacggctacgtgcccgtcgtgatggacgggctcggcaagtgggccgtcgaacacgacttcgacgtgctgctcaccgtcacggcgcacgtagaggcgcaccgcgactcgtcgttcctgcgcagcatgtcgctgatgtccagcgcgatctcgggcgagccggccgacgtcctgcaggagcgctacgccgccgactacgccgagctgggcggctcccacgtggccttctacgagctgtgcgcgcggcgcggcggcggcacggcacggctcgccgacgtctcggcgaccaagcgctccgccgaggtctggttcgtctgacgccacccaccgaccggacgggaccgcgatgccgaccacactcccgaagaccctgcgcgggtcggacggcgcgctcgccgagcagccgctgctcatctacgtgaatgtgccgttctgcaactcaaaatgtcacttctgcgactgggtgaccgaggttccgctggcggatctgcggctcacgccggactcgtcgccacgccgccgctacgtcgccgcgctggtcaagcagatcgagacgcacgcgccgacgctgggcggactcggctaccggcccgaggtcatgtactggggcggcggcacggccagcatcctcaccatcgaggagatcgaggcggtggctggggcgctggcggcgcggttcgacctcggcggcctggtcgaggccaccatcgagggcagcccggagtccatggaccccggcaagctgaagctgttccgtgcgatcgggttcaaccggatcagcatcggggtgcaggcgttcgacgacgcgcgcctgcgccgcatcggccgggtgcacagcgccgagcaggcggagcgcgcggtccggatggccgccgacgccggcttcgacaacatcaacatcgacctcatcgtcgggttccccgggcagggcgccgacgaggtgtcgcgcacgatccagcacgcggtgacgctgccggtcaaccacttctcggtctacccctaccggccgaccggcggcaccgtgatgcgccggcaggtgggccgcggcatgggccggatcgacgtggaggagcagctccggtcgtacgaccacgcccgcgacctgctcgcccggcacgggttcgaggagtacgccaccgcctacttcggcgctccccgctgcgagtccgacgaggtctactacaagctcacgatggactggatcgggttcggctccggggccaactccctcatcgggacccgcttcctgctgaacgaccggggcgggctgcaccgcttcagcacggccccgcaccgcttcgactccgacaccccggcctcggccccccacctgacacgccacttcctggcccaggcgctgaccacggtcgacggcatggacgcgcgcaccttccagctgcgcaccggccgttcgctgcgggcggcgtgcgaggagcccgcggtgcgccggatgctcgaacagatcaaccgccgcggcaggctgatcgtcgactcacgcggcatccggctgcaccgcgacgacatggcctcggcctacatcaccatgaacagcgtcgatctgtacgccgcgaccgagcagcccggcggatgaccgcggtcctgcgcgcctgctcacacagtcttcgtgaacccgtgtgcccttcccacgtggggacgggcgtgccggtcgaccgccgcgggctcgcgaaggaatgagggtcacaacggaaaggagatgagcgagttggaatccaagctgaaccttagcgacctgcccatggacgtcttcgagatggccgacagcggcatggaggtcgaatccctcaccgcaggccacgggatgcccgaggtaggcgcttcctgcaactgcgtgtgcggcttctgctgctcctgcagcccgtccgcgtagcagtcaaggagtgccgccggcccaggccggcggcactccgcccggggtgaggagacagatcatggaggtgggccgcgaaatcgtggtccgcgtggcggggctgcccgccgccgtgctcgccgacctgcgcctgccgcacaccgccgagctcgtcacgcacctgtccgctgagcggcggcggctcgccgccgaggcggctgcgctgtccggcgagctgttcgacctgatcggcgccgccggctccgcccgcgccgccctggtgggcctgcggcgcgcgctcgcccccgggcaccgccccccgtcgccgcgactgatcgggctgtgtcccctgcccgccccgctggccgagcgggtcaccgcctgggtgcgggcccggtacgactgggaggaccggcgccgcgaccttgccggcacgctggacaaggagcgcgccgacgcgctcgaccgcgtacgcgccgcctgcgtcgccccggcgttccggcggggtctggcgctgtccggtggggagctgaccagcacgctggagcgctggctcgccgaccccggccgtgcgccccggcaggggaaggtgctccgcctggtcaagtacctgacccgagctgcggccaagaccagcccgtacggctccttcatggtcagcgcgctggcggggcggcccggcgacgaccctcccatcccggagctgctcaccgtggccgagccgccgggggcgttcctcgacgccgtcggcgacgcgctgctcgccgacccggcactggccggccaggtgccgctgcgccccaaccccagcctgacgtggaccgcccaaggactgcttttcgtccggaccgtccgcgcggcggcgggagagcaggccgggccgaaggaagagatcgccaccgccgggcgcgccgccgcgctggagctgtgcctgcggcacgccgagtcccggcccaccgcgccgcagctggcggagctgctggccgaggcgggggcggatccgggcgaggcggccgcgttcgtcgaccggctggtggcggcgcagctcctgctgccgtgtccgccggtgcacgacgacgatcccgacccgttcggtgcctgggcccgccaggtgtgcgccccggagctgcgcgagctggcggccgcgtcgcggcccgtggccgccgccgtggactcgcccggccagcgccgcgccaggatcgccgaagccgccgccgccgtggcggaccggctcggcatcgacccgcccgccgatccggcgcacgagcacgacgtcagcaccggccgtccggcgccgccaccgctgcccgcgggcgtgctcgccgacctggacgcggtccggcgctggctcgccgtgttcgactggaaggtgcccgtccgggtgggggtcggcgcgttctgccgcgaacgtttcggctcagggtcccgcacgcccttcctggaggcgtgccgggcggcgacggcggcgctgccgcacctgttcggccccgccgcgatgccgtggttcctcgacctgaccggtgacggtcgcctgcgtgagctggaccgtctccggacgcgcgcccgcgatctcgcccgctccggcgtcctcgaccggcggcgggtgctggctgacacggccgactggcccgcctggctgacctggcccgcctccactggtttctacctgcagaccctgcccggtggggtggtgctgaacgcggtgcatgccgggcacggccgcgcggcgggtcgcgtccaccacctcctcgcccgggcgggcgccgcgccgccgcgcccgccccgctccggcctgccgcgtgccgagttcggcggcaggttcggctccgcgctcaacacccgcacgccgagcacgcggtacgagatcgaccaccccggcgcgacgtccggccgcgacccccggcaccgtgtcccgctcggcacgctcatggtggtgcacgacccggacaccgacctggtccacctgcacagcgaccggctcggccgcgtcgaaccggtgcacctcggcatgatgggcgagctggggctgcccgccgtggccggtttcctcgaacgcgccttcgcgcccacctacctgttccacccgagcgtgccgccgttcatctcgctgcgtgacctggccggcgcctcctcggcgcggcgcttccccagggtgagtgtcggcgacgtggtcgtgcagcgcgcccgctggacggtgcccgccggcctggtgcccgcgcgtaccggggcggacggtgaccacctgctcgcgctggccgagtggcggcgcgagcacggcatccccgagcggtgtttcgtgcgcggctggaagccgggcgcagcgctgggcaaggcgcgcaagccgctctacgtcgacttcgcctcctggcatctggtcgccctgttcgaacgggaggcgcgaaccaacgccgccctggtcatcgacgaggccctgccggacccgctggccgacggcgcccccgcgcacgtcaccgagtaccacgtcgagatcggcgaccgggagagagagcaatgaccgaacggcccggctggttcgcggcgcatgtgcactaccgcggcgacctcgacgcgctgctgcgcgaagcggtcgcgccgctggtgcgcgcgcttgccgccgacttcttcttcctgcgctactgggacgggggcagccacctgcggctgcggctgcgcggcggggaccaggtcgcggtcgcccggcacctggacgcctaccttgccgcccaccccgcgccggagaccacctcccaggaggagtacgcccgcgtcgccccggtgctggccgcccgcgagggcatgacgggccacctgacgacgctgcggccgaacaacaccgtggagttcgcggcgtaccggccggagaccgcgaagtacggcaccggcgacgcgctacgcgccgtcgagcggcatttcgtcgagtccagccggtacgcgcttgacgtccttgaccgccgcccgaccggcaaccaacgcgagctcgcagtgctcggcgtcctgctgctcgcctggtacgccgcccgcccactcgatgagccgcctggtgagccggtcgatgacgagctgatcggcgcggtcgaggcgctgtgccggggctggcgcggcggccgggacctgcccgaggagctcgtggcggaggagtacggccgggtgcgggagcgtgtggccgggctggccggcgcgctgcgtgacctcgttccggacccggacgcgcccggcagcagcatgcgcgcgtgggcggcgacgttcgaccggctcgccgccgcgctgcccccgcccgaccggctgcgggtgctcgacacgtgcgcgcatctggcggccaaccggctgggcgtgtccatggcggccgaggtgcggctgcggctgctcgccgcgcgggccctgcgcgaggtcgcgcccgtggggaggcgatgatgagctggcgacgcgtcgacgtggcctaccacgatcccgaccttgacgggctgatcctggccacccggccgctgctggccgggacgcccggccgcggctggttccagcggcactgggtccgcggtccgcacctggagctgtggttcgacgccgcgcagccgtcgtgggagcggatccgcgacgtgctggagccctggctgcgcgtcaacccgtcgcgcgcccggatcgaccgagaccggctgctggcgcagcaccggcacctcgccgccgccgaacggatcgacgagccgctgctgccgttctacgccgacaacacgctgcaccgggccgctccgcgctcccgcgcacacgtgctcggtggtccggcggccgaggagctgttccacgacttccacaccaccgccagcgccgtggcttacgacgagctcgacgccgtgcgggcgggagagtcgcggctggtgatggcactggacctgatggtcgccgccgcgcacgcgcacgccgagggcggcgtccgcggcgggttcgtgtcgttccgctcgcacgcggaggcgttcctggcgagcgcgcccggcctgcgcgagcgctgggacgccgagtacgccgcccgggcgggggcgctgcgagcgcggatcaccgcggtggtcgccggaatcccgcgcggccgggcgtgggccgggctgctcgaccggttcgcggaccgcggcgacgagctgatcgcctccggcgcgctgctggtcgagcccgccgggccggacgcggtcgcccggcccgacaccgccttccaccgggcgctgcgcggcaaccggacctggcacgaggaggtgctgcgctcagcaccgttccgccgctaccggctgctgctgaacctgacctacctgcagctttcccggctcggggtgaacgccgtgcagcgggcgctgctgtgccacttcgccgcgtcggcggtcgagcaggagtacggggtgtccgccatcgagatcgccatgggaggcgcgtgatgtcagtacaggtcgagcccgcagtgacgcaccggtgggcactgcggcccggcgtctaccgggccaccgcgccaggcggcgatctcatgctcgccgcctggccgcacgccacgccgctcggccgggccacccccgagctgctggccctgctggacggcctggccgccggatcgctgccgctcgacgagcccggcgacctcgcgcccgccgtcgagctgctgcgcgcgggtggctggctgcgcaagacggtggcgtacgcgggacgcgacctctacaccatcacccccgtcgccgcccccaccgacgcgccgccaccgccgcgggacgaaccggcgctgtcgcggttcgcgctgctgcgcaacaccgcggacgggctggtgctggagatgcccggctcctggtgcgacgtccacgtgcacgacccggcggtggccgcgctgctcgccgacccgtccggcgacggcggcctgcccgccgaggccgcggccgcgctgcgcgccgacctggccgccgccggcatgctgatggagcgggaggagccgttcgagcggcggcagtggagcacccacgagctgtggttccatgaacgcagccggctcggcaaccggggctggttcggcgcgcacttcggcggcacgttctgggcccgcggccggcacgacccgccgcccgcgcggccgtccccctaccccggcccggccgtcgagctggcccggccggacctcgcggcgctgcggcgcaccgaccccacgctcaccgccgtgctggaggaccgcgaaagcgtccgcgaccacgacgacgacgatccgatcagcgctgagcagctcggcgagctgctctaccggtgcgcgcgggtgcggctgctgcgtcacctggaaggcttcgagtacgccggcaagccgtaccccgccggtggctcggcgtacgagctagaggtgtacccggtcgtgcggctggcccgcggcctcgaccccggcatgtaccactacgacgcgcacgaccatcggctgcgccaggtccggcccgccggccacccggcggtgcgcaggatgctgaaggtcgccacggagtcgtccgtcgccaaggcgccgccccaggtgctgctggtgatcagcgcgagagtcggccggatcctgtggaagtacgaggcgatggggtacgcgctcatgctgaagcacgtcggcgtgctgcaacagacgctgtacgcggtcgccaccgccatggacctggccccgtgcgcgctcggcagcggggacgatctcgccttcaccgccgccaccggccgggaccggctggtggagtgcgcggtcggcgagttcatggtcggcagcaggcggaaggagcccgcggcatgggagctctgaacctcctcctgcgccccgacgcgcactacgccgaggtcgacggcggcgtctacgtgctgagccaccagggcgagaccttcatctcgggcacctcggtgcaccggtggctcgaccgcctcgcgccgctgctcgacggcacccgcacgctcgaccagctcaccgccggcctgcccgccgaccgggccggcttcgtggccagacttgtcggcacgctcaccgaacgcggcctggtgcgcgaggtcggcccggcggtgtcggacacgctcaccgacgccgaacgcggcgagcaccgtgccctgctggcctacctgggctacttccacgactcgcccggccgcgtcctcgaggacgtgcgcgacaccccgaccgcgatcgtggggtccggtccgctggccgccgcggtggtccgcgcctgcgccgccgccgggctgcgccgggtggagatcggcgaggaggcgggcgcggcccgggtggtcgtccacgtggccgagcgcgccgagcccgaacgggcggcgcggctggaacggcggtgcgccgccgagggggtgctgctcgcacaggtcatgccggggccggacggcatctggtggcagcccgcggcccgcggcggctgctgggcgagcgcgtggcgccgccaccgggcgctcgcgggcgtggatcggacgggcacgccgctcgatccggtggcggtcgcggtggtggccggccaggtggcacacgacgtcttccgcgtcctcgccggactgcgcgaggaggcgccgccccggttggtggtgctcgatccgcggacgctggcgagcaccgcgcacccggtggtggcgcacccgttcgagctgcccgccgcgccgctggacgaggcggcgttcctcgaccgggtcgccgcgctgcgggcagcgcccgcgctgagcgaggcggagttctcccggcgggccaaggggctcatggactcgacagtggggctgttcgccgagatcgacgagggcgacctcgcgcagttgccgctgcacgtcacggcgaccacggtggccgacccgtgcgggctgatcggctccgcgccgcggcccgtggtcaccggcgcggggctcacgttcgaggaggcccgctaccgcgcggcgcgcgccgcgctcgccctggccggcactctcgcgctcgaccggcggaggctggtcgacggccgggtgtgggcgtacgaccttgcggacggggccgcccggctggttccggccgacgaggtcttcgcaagcacggcgggcgggagcacggcgggcgtgccgctcggcgcggcggcagcctactcgtgggccgaggcggtcgcggagggcctggtcgcgcaggccgccgcgctcactctcgccggcgccgggcacgcgggcgagccgtatggacgggcggacctcgccggggcgcctgcgtatctcctcgccatggtgcgggcgctcggcgaggagctgaccgtgcacgacgtcaccgggccgctgggcgtgccggtcgtggtggggacgctgtccggcggggcgaccgcctgcggcgcgggcatgaccacggacgccgccgtcaccgcgtgcctgcgcgacctgctgctgctgcgccaggccgagatcaacgaccagcccgtctacgccccaccatccggcaggcccttgcccgggcatctacgcgggaaccacccggcgcccgccgtgcccggcacggacgccgaccggctggcggcccgcctggccgagcgggggcaccgccctctggcggtgccgctggatcatgaccaggccgtgcacgcggtcctgcccttcgccgtccgggtggtgtgccgatgatccgcctgcccgaccacggcgagcttgccgtgccgatcggcgacgggccgatgctgccgctgcgcaccgaactcggccgggtggtcatcggcccgctggagcggcccggcgtgcccggatgccgggcctgcgtgcgcatccgcgcggcgcgggtccagcccgacgcccgcaaggccgaggccgtacgcgcccggcacgcccccgcgcccagccagtggctcaccccgctcgccaccgacctggtgcgcgtcctggtcgcgagcgagagcgctgccctggcagccgacaccgacccccgcaccgcgggtgccgtgctggagatcgacctcgccacgctggaaatcaccaggcaccggttcctgcccgacccgctctgcccgcactgcggcggtctgccgccggacggcccggccgagctgaccctgcggccccgccgcaagctcggcggcagtccccggaccaggccggtcgagctgaacgcgctgctggagacgtacgtggacggccgggtcgggatgatccggccgctgaagtcgggcgtgcagggcggactgacggtggccagcgccatgctgccgatccgcgcgggccacggcctggaaccgggggtcggccgtacccgcagctacaccgcgagcaggctggtcgccgtgctggaggcgctggagcgctatggcggcgtcagccccggcggccgccgcaccagcgttaccgccgcctaccgcgacatctccggccacgccgtccatcccgacaccttcggcacacaccccgaggagaactacgacaggcccggcttcgccttccggcgcttcaccgaggacaccgtgtgccggtgggtgtggggctactccttcgccaaggcccagcccgtgctggtgccggagaaccaggtctactactacgccagaaacctgccggacggcgagcagccgttcgtcttcgaggtctccaacggctgcgcgctcggctcgtgcctggaggaggcgatcctgcacggcctgctcgaggtggtcgagcgggacgcgttcctgctcacctggcacgcccggcgggaggttccggtgctgaaccccgcgctggcgagcgacccggtcctgcccatgcaggccgccgcgatcaccgccgagaccgggcaccgggtgctgtgcttcgacaccaccgccgagcacggcatccccagcgtgtgggcgatggccgtggacgtcgaacaccggtcggaccggcccgccaccgcccacgccgccggcgcggcgctcacgctggagcaggccgccatgaacgcgctgagcgagctcggcccgctgctggccgacgtgatccgccgctaccccgaggagcgggaacgggccgaggccatggtccgcgaccccgagcaggtcgccaccatgcacgaccactccctgctctacgcggttcccgccgcggccgaacggctgtcgttcctcaccggccgcgccgacggcccgcggaccggcttcggctcggcccggttcaccggcgacgacctcaccgccgatctgcgggccatgatcgacgcggtgatcgacgccgggatggacgtggtggtcgtcgaccagaccacgccggagcacctggcgggcgggttccgctgcgtgaaggtgctggtgcccggcgcgctgccgatgacgttcggccaccagtaccggcggctggacaacctgccgcggctggcggccgcgcgtaccaccgacccgcacccgttcccatgagcgggcgcccggacagcggaaaggagccgtcgtgtacgtggtgatcgtcgcgttcgacctgaaggagtcctccgtcgacttcgccgaactgcgcgcctgggtgcgggaccgggcggcggacgactactcccggctgccggggatgcgcttcaagacgtggttctccgacgagcgaaaacggctctggggcgcggtgtacctggtggagtcgatgttgtcgttcgaccgggacaacctcccgctgctgcccgacggcaggaccgggccggtcggcacccggccgacgtcggtcatggtgctggagttggaggcgttcgtcaccgggccggacggcctggacgggatcgaggcgctcgcccggcaagggctgagcatgacgggaggcggccatgaccactgaggcgacgactgcggtgaccactcgtgcgaacgccggagcgctgtccctgccggcgcgtggcacggaccggccgctgctgctctacgtcaacatcccgttctgcaactccaaatgccacttctgcgactgggtggtggacgtgcccgtctccgacctgcggctcgcccccgcggcgccgcgccgggtcgactacctggccgcgctgtgcacccagatccgcacgcacgcccccgccttgcgggccgccggttaccgcggtgaggtcatgtactggggcggcggcaccgccaccgtcctcaccgcggcggagatcgagcaggtccacgcctgcctcgcggccgagttcgacctgacggcgctggccgaggcgacgatcgagggcagccccgagtcggtggacccggccaagctgaggctgctgcgggacctcggcttcgaccgggtgagtctcggcgtgcagtcgttcgacgagaccaggctgcgccgcatcggccgcgcccactccgcgggccaggccgtgcgcagcgtcgaggacgcgcacgcggcgggcttcgacaacatcaacatcgacctgatcgtgggcttccccgaccagtcgctcgccgaggtggagcagaccgtgcggcgcgccctcgacctgccggtcaaccacttctcggtttactcctaccgagcgaccgagggcacggtgatgcgcaggcagatcgaacgcagcggcacggccatcgagctggagcatcagctccagtcgtaccggctggcgggcgagatgctcgcggcggcgggacatcccgagtacgccgtctcctacttcggtgcgccgcgctgcctcgccgacgaggcgtactaccggctcagcatggactggatcggcttcggcacgggcgccaactcgctgatcaaccagcgctacctgctcaacggccgcggccggatgcgtgacttcaccagcaggccgggcgagttcgaggtggacctgccggccggagtgccgtcactcaccgtgcagtggctgcccagggcgctgggcaccgccgagggcatcgacgcggcgaccttccagcgtcgcaccggcacgtccctgcgtgcggcctgcgaggaacccgagctgaacgcgttcctcacccgggtgaacgagttcggccggctcgtcgtggaccgcgacggcatccgcctggccgacgagaaccgctcggacgtgctgtcgcgcacgttcgccgcgatgggctgggtgtcctgacgtggaggcgatgcgcacgctgctcgcggccgccgacctggtcacgccgatggcgctgcgggtgtcggcgacgctgcggctcgccgaccacctggccgccggcacgaccggcgccgccgagctcgccgcggccgccggggtcgacgaggcggcgttgacacggctgctgcgctacctggtcgcgcgcggcgtcttcaccgagcccgcgcccggtgagttcggcctcaaccccgccgccgaactgctgcgcgacggccgccccgaccggctgcgcgactggctcgacctcaccgggccgatcggccgcgccgacctggcgttcgggtcgctgctggacgtcgtacgcaccggcaagcccggttacccgatgatccacggccggggcttctgggacgacctcgccgccgagccggcgctcgccaccgcgtacgacaccctgatgggcggcaagcgccagtgggccgcgaccacgctcgccgccctggactggagccgctcccggcacgtcgtggatgtcggcggaggcaacggcacgctgctgtcgtgcctgctggccgcgcacccgcacctgcgcggcaccgtcgtggaccggcccacctccgcgaaggccgccggagcagtactggcctcggccggggtggcggaccgtggcgagtttcgcgcgggcgacttcttcgagccgctgccggtgcacggcgccgacacatacctgctcagctcgatcctgcacgactgggacgacgccgcggctacggcgatcctgcgccgctgcgcggaggccgccgcgccgggcgggcgggtgctgctctgcgagctggtggccatggccgggccggaccggcggacggtgacgcacatggatctgtgcatgctcgtgtacttcggcggccgggagcgcaccgccgccgacttcgccgcgctggccggggcggccgggctggagccgcgctcggtgacgccgctcccgccccacgactggggcaacgccctcgtggagtacgcggtccccgccgcccgctgaccggaaaagacccctgccgacggcgacggtcagcgcggcgcggctccgttgatcgcgcccgcctgggccgcacgatgagctggagcgctggtcccactcgaccgcaatcgccgtggaggtgtgggcacttcccggcgagccctgtccggcgacgtcgcggagctcgtccatgccacgatccccgacgtactcgacggctcgggacgggacggatcggtgcggtgcgcgggcatcgcgctcaccgcgggcgcccgcgggctgcgcctgaccaccagccacgacggctccggctcgccgcgctggggttcaaactcactacccagggccggtacgggatgagtttcgtccgcttctgtgaggaacgcggcgctgaccgggtggcggcgcgccggttcggcctacatcaaggagctgatgcccgacatctggatggcaccatcgagccggggcaaatgttcgacgccacgaccgatgcgatgtcccgcgaccggccgtcgcggtgaggcgtgccgatgcatcacgacgagctcggaataccgagtctgcgtcggggtggatttgtggctggtcgatgagtttgggatgctgcgcaggtctgcctgccgataccacctgactggaaggggatcaccgcgatggcgaaggtcatctctacgttgttcatctctgctgacggtgtggccgagatcgaccccgattggcacttcccgtacttcgacgagaacatgggccgcgccgtcggcgaggactatgacaccgctgacgtgctgttgatcggtcgcgagaccttcgacagcttcgccggagcgtggcccgaccgcgaggcggcgggtggggacgacgcaccgttcgccaagcaactcggggacatgcgcaaggtggtcgtctcccgccagccgctggagttctcctggcgcaactgcgaactgatcacgggcgatctcctcgatgccgtcacctcgctcaaggccgatgccggcatcaggggcatcctcatccccggttcgatctccgtggtgcagcagttgctcgccgcagggctggtcgacgagttgcgcctgctggtgcacccggtggcggcgcgtaagggccgcaggctgttcgacgacggcgacgcgccgtaccacctgagcgtgacggcgacggagacgtttccgacgggcgtgatccgcgtgatctactcgccgaccgaggcacccaccaaagtcggctacgacgaggtcgaggcccaggtgtccaccggggaatag

SEQ ID NO: 63 - DNA, 15586 bp, nonomuraea sp.
ctaggtgggtgagggggttagggggattacctcgttgggggtggtgtcgggggtggggatccagttgagggcgatgaaggtctgggcgatgtcgtcgcggtggaggcggatgccgttgtggtcggcgatgagccggccgtggttgttcatgcgttccaggtacgtcatgacctccgggtgagagcaggccgtgcggagggggacgccggtgcgttgctggtagaggcgggcgtccaggccctcggcggttgtcagtgcctgggacaggaagtggagggtgagctgtggggagtgggccggggcgttgatgtcgaaggccagcgggttggtgttgtagtgggccagcttgcccttctcgtagctcaggtagcggcggccgatcagggagttggcgcccgagccgaagccgatccagtccatgcggagctggtagtacgcctcgtccgactggcagcgggggtggccgaagtaggacatggcgtattcggggaagccggctgcttccagcagttccctggcgatgccgtacgcctggagctggaggttgaggtcgagctgggcgccccggtggacctgcttgcgcagcaccgtgccggggctggcgcggtaggggtagatcgagaagtggttgatgggcagggtcagggccgtgcggaccgagtcggcgacctcctggccggtctgcccggggaagccgacgatcaggtcgatgttgatgttggtgaagccggcctcgtgggcgtcctggacggcggtgagggcctggtcggcggcatgggcgcggccgaggcggcgcaggcgcgcgtcgtcgaaggactggacgccgatgctgatccggttgaagcccagctcgcgcaggaggcgtagtttgtcccggtcgagggattccgggctgccctcgatcgtggcctccctgaccgtgctcaggtcgaactcggcggacagcgtgccgtacagggactcgatctcgtgggggccgaggatgctggccgtgccgccgccccagtagacgatgtcggggtggtagtgctcgcgcagggcgggggcctggacgcggatctgggtcctgatggcctccaggtacgcgacgcggcccgggctctgctcgcccagccgcaggtcgcgtacggggacctggaccacccagtcgcagaagtggcacttggagttgcagaaggggacgttcgcgtagatcagcaggggcttggtcatgaggaacctctcacgtgcatctccctgacgccgaagatctggctctcgtagaaggagcgcttttcgggacggagctcggtgaagcggctcagcaggcgctccagcgcgatgcggccctccaggcgcccgagctgggcgcccaggcagaagtgcacgccgtggccgaacgcgagctggccggtgagctgccggtccaggtcgaagcgttcggggtcggcgaagtggcgttcgtcgtggttggcggacaggagccaggccatgacgaacctgttggccgggatggtctcgccggccacctgtacgtcctgcgtggtcaccctgcccgcctgcatgaacggcgagcgcaggcgcatgctctcctccagcgcctgggggatgagcgacatgtcggctcttgtccggtgggcggcctcgggggcgtccgccaggcagagcacggtgttgccgatcagcacggtggtggtgacgtgcccggccagcaggagcaggctcgcggtgttgacgacctgctccgcgtcgagccgctcgccgtcgatctcggcatggacgaggcgggtgagcaggtcgtccctgggacgggtacggcggtccgcgcagtgctcgcgcaggtagtcgttcatctccgccatggccgccgccacccgcccggccagcgacgggtcggcgaagtcgggcacctggagcgacatgagccggtccgaccaggtgcggaacaggtcgcggtcggacggcggcacgccgagcagctcggcgacgacggtgacgggcaggggataggccaggctctcgaccaggtcgaagtcagggccgggcagggcgtcgaggagctcgtcggcgatcgcggcgatgcgtggttcgagggccgcgacggagctcggggtgaaggccgcggtgatgagcctgcgctggtggcggtgttcgggcgggtcgatcatcgtgatgccgccctggccgaacggctgccgtcccatcagcctggtgcggtcggaggagaaccgctcgtggtccgccagaacggcgcgtacgtcgtcgtagcggaagacgtggtacatcccataggaatcccgccagaccgggtgcgtgtcgcgcatctcgcgcagccaggcgaacagggacgtcccgccgtccgtgaccgaaggtggccttacgggggccgtcatcggggcgcccgcttgacgcgtaccggcaggctcttgacgccgaacatggggtcctcgtagtacgacagctccgcgtccgggtcgatctcgacctgctcgaaccggtcgaacaccagctccagcgccaccttcccctccaccctcgccagcggcgcgcccaggcagaagtggatgccgtggccgaaggcgtactggcggttgggctggcggccggggtcgagccggtacgggtcggggaactggcgctcgtcgtagttcgcggacagcagccagttgatgaccatggaaccggccgggatcggcgtgccgtcgatctcgacgtcggccgtggggagccgggcggcctgggtgagcggcgggcgcagcctgagcacctcctcgaaggcgggtgcgatcagctcccggtcggcccgtacggcgctctccacgccgggcgcgtcgcggaagcacaggaacgcgttccccagggccatcgtgctggtgatctgcccggcgagcagcagcaggcaggcggagttgacgatctcggcgtcggtgagccggtcgccgtccacctcggccgcgaccagccggctgacgaggtcgtcctgcggacgggtgcggcgctcgcgcacgtgggtgaggacgtattcccccatttcgcgcatggcctgaccgaccatccgcccgatctcgatgtcccgcgggtcctcgacgtgcagtgcgaccagccggtcggcccaggtgcggaacagctcgcggtcgtggatcggaatgccgagcagctcggcgatcaccatcacgggcagcgggtgggcgatcttctcgaccaggtcgaactcgtcctcgtcgatctcgtccagcagcgccgtggtgagctcggcgatccgcggcaccatgtccgacgacattttcggggtgaacgcctggctgatgagtttgcggagctttcggtgctccggcgggtcggtgagcatgaggctgcccgcgctgaacggatcgccgccgggccggagccggttgatgtcagaggagaacgccgcatagtccgacatcacccgctgtacggccgcatgccggaaaacgtggaagatgccgaatgcgtcgcgccagaccggttgttcgtcccgcatcgtgccgagccacttgaggagggtctggccgccgtccttgatgcgcggccgggagtcgatcggcatcgtcatcgtcgggtccggcgggacgtgtgtctcgttcacaattgactcctcagacggaggcggccaggggctgggggatgagtttcgcccggtatttcgccgcggcgccgtcgaagatctcgacggtacggcgggcggggtcggtcagttcttcggccgggacctggtcagcaggttcgaaagagctcctgatgaggccggccaggctgtcggcgtgcgccgccagatcgggtttcgccccggcgatctcggcgtgcaggtgaagtttgatgaacccggaaagcagggcatcgagcgcgaaaccgtgggacgggtcccacattccgctctcgtggagcatccggagataatcgcgaaatggcgtgaacgtggtgaagcggtaacactggaagccgagctgtggatcgtcgcaggcccttccgagcgtttccttgtcataggtgtagagtgctcggaagagtggatacttgaacacgagtgtgaacagctcgcgtgagaactcggagagccgcgcgacggcaggacgaagcaccgctattcgcgaaatatcctggcagactccgacaatttcctgaatgacgagcgttcttatgagatcgtctttcgtgtcccagctgagataaatggtgccctttccgatgtccgcccgccgggcgatctcgtccatcgtggtccgcttgtagccggatcgcaggagcaggctcctggtgacgtccagagtccggcgtacgcgatcgtcgctggtcctggcacccgtgctgcgtgtcgagacggtgctcatggctgggtggctccttccccgtaagagctggtgcgcatgaaccggtattccctaccggctagggggattttggtgggagatgtgctctgggcccgtcagagctggagatttcgaagatattcgaaatgctcagatttcaacagtcaggtgccccctccctgactggctgaccgattttcgattttagtcagccagttcgcattcttgaccgcgttccatatgtacacctagcgtcggcgatgtcgcaagggtgttcctataccaggcgatgacctccgtgcgatgcacatcagtgcgtttgcctggaatgtcgatggggcgtttacccccggaaggagagtccgcatggacctcagcgatctgcccatggacgtcttcgaactcgccgatgacggtgttgcggtcgagtcccttactgctggtcacggcatgaccgaagtgggagcttcgtgtaactgcttctgctacatctgctgctcctgctcgagcgcctgacgcggtacgcgggcgtcgccacggcggcgcccgcgcctgttgccttttggcgccaataaagcgaatggggctcatgtaatggcggttattgagcgggaagaaagcgacggcccgccggtcgctgtccgcacgtgcggggtcgcggccgcggagctggaatccctgcgatgggacgagtcctatgcgctggtgacccggctgctcgcggagaccgcccgggtggcggcggacgggcggcggctggccgtggcgctcggtgacgtcatcggagggctcggcccgtccgggtcccggcccgtcctcgtggggctgcggcgggcgctgcacaccgggcgtctcccctccgggcgggagtgggggcccgaggccggggcggcgctgccgtacgggctgaggggcgaggtggaggagtgggtacggcgtgcgcgcgagtgcgccgggctgcgcgcccgcctgcccgaggtcgtcgcccgggagtccctggagaaggaggaacggctgcgcgccgccgcggccgaccccgccttccgcagggggctggcgctggccggcggcgagctggcggtcgacctggagacgtggctcgcggaccccgcgcggcgccccaagccgcagaagctgctcaggctggccaaatacctggtcagggccgccgtgaagaccagcccgtacagcacgttcacgagcaccgggcgggcggtgtggggcggtggcgagcggcacgtggagcgcgtggtgccggtgctggaactcgacggcgtccagacgttctcggacgatcgggtcagggtgaatccgtcggtcaccgtcgcgggcgggacggcggagttcatcgggccgcctcccggggaggcgctggtgtccatcggggtgacggaggcggtggccgcgtgcctgcgcgtcgcggagggcggggagtgggtgccgcggggccggttcgcggaggctctgggggctgagcctgccgcagtcacgaaatttctggacaagttgcttgcggttgggctcctggaggcccggccggacggcgacccgcccccgcacctgctggacgggatccggcgcacggaacgcgccaccgacccgtccaccttccgccacgaactcggccgcctgatcgccgccgcccgcgccgccgcccgcgccaccgcccccggcgctgacacggccaagaacgccgcccctgccgggcgcgccacccgcggcgatggcggtgctgatgccgagcgcgccacccgcggcgatagcgatgctgatgacgggcgcgccacccggggcgacggcggtgctgatgacaggggcgccgcgcggggcgacggcggtgtggggcgggcggttgtgcacgaggtcgcggtctgcgtcgagcccgtggcgaggctggatccggcgcgatggcgtggcgggcttgccgatctcgacgtcgtacgccgctggctggccgttttcgacgccaagcaccccatgcgcctcgcggtcgccgcctacctcaccacccggtacggccccgacccggccgtccccttcctcacgctccaccgccacatccagcgcgagctggccggcaccggcgcggcaggccgggagttgcgcgcgttcctcggctcgtcggccgcctggacccagccgctaggcacctccgccctgcccaggatcagggagctggagcgcctcagagccgaggcgagaagcctggcgctggacgcggaggaccccgacggcacctgccgcgtcacccccggacagctggccgcgcagctggagtcgtggccgtcttggatcgcgatccccgcctcctcggcctgctacgtacaggcggtggccggcaccctggtgctcaacgtcgtccacggcgggcacggccgagccctgcgcaggctcgaccacctcctcgaacgcgccggcggccaccccgagccgccccggatcgaggacccggacggcgcggtctacgccgagttctccggcgacctgggctccacgctcaacgcgcgcccgccgagcacccgatacgagatcgactacccccactcgcccggcacgcgcccgcccgacctgcggctgccgctcacggacctgcacgtgacgctctgcccggacagcggcctgcccgagctgcgttccaagcggctcggccgccgggtcgtgccgttgcatctcgggctggccgccgagttccggcttccgccggccgcgcggttcatcgaacgcgtcttcggccccggctacctgctccatcccagctcgccgccgctggtgcggatgggccgggtcccctcggaggtcacccggtatccacgcgtcgaggccgggcgcgtggtggtgcagcggcgtcgctggctggcgcctgcggccacgctgccggtgcgcgcgaagggcgagggcgacgcggcgtacctcacgcggctgatcgcctgggcggacgagtacggcgtaccgcaccgatcgttcgtccgcgcctggccggagcagaccggcgacaaagggcaggacaaggcgcgcaaaccgctcttcctggacctggcgaacctgttcctggtcaagaacttcgagcggcagatacgtggctgcgcgttcgcgctcttcgaggaggcgctgcccgacccgggaccggagcgggtgaccgaatacctgatcgaggtgggaggccgatgagctgggtgagcgtgcacgtcttctcgcaggggccgctcgacgacatgatcacgggcctggccgggccgctcctcggcgacgacggcttcttcctgcgctactgggagggcggcccgcatctgcggatcagggtcaggtcccacccggcggagcaggtcgtcgagcgggccgaggcgtatctgcgcgagcacccgtcagaggcggtgatcgacccggaggagtacgccaggctcgccgcgaccctcgccgcccgcgaaggcgtccccggctatacgcggcgcctctatccgaacgactccgcccacctcatcccgtaccagccggaacacgaccgctacggcacgggccgctcgctcgacgccgtggagcggcacttccacgactccagcgtgatcgcactccagctgatcggggcggggctcggacacgagcggcggagcatggcgtggctgaccatgctgctggtcgcctggcaggtcgcgggggagggcagggcgttcggcccgcccgctgacgccggggttcaggtgccccacgagcggatggcggcgctggtcgagcacgccagggcggtgacacaggagcctgcgccgagcgagccccgtggcgtggtggccgcctggtacgcctcgatcgcgcggctggtggaggaactggcggcggcggggttcgacgcggcgcggacgggggcgaccgtcgatctggccgcgcatctgctggccaacaggctggggatccgggtgcaggaggaggcccggctgcgtcacctggcgtccggggccacgagggagtcggaggtgggggtcggatgacctggacgcggctgcgggtggactatcacgacggcccgatggacgacctcatcctggacgcgctccgcccggcctggcatgaaatacggggatatttcctgcgccactgggtgtgcggcccgcacctgcggatcttcgtggacggcgacgcgacgccgatcgtccgggcggtcgagcgccacctggccgcctgcccgtccgccggcaccaccgacccgcaggcgctgctccccctgcacgagcgcctggccgagctggaaggcgaacgaggcccgctgctgccatgggcccccgacaacaccgtcaccgccgaacccccggggctcgacaccgagctcgaccggttcctcgcggacttctacgccgacaccaccgaggccgcgttcgacgccctcggccgggtcagagccggcacgccgctgccgggcatcgccttcgacctggtggtcgccaccgcgcacgacctgtcggagggcggcctgcccactgccaggacctcgctgcgctcgcacgccgaggcttacctgtcgcgcctgccgggaggcgtccgcgccaagtggcaggcgcactacgaacgcaaccaggagccgctgaccgccaggatcaaggccctgaccggcgccggagagcccggcgcgtggctgcgtacgatccgggccacccgcgaccggggccgtaccctcatcgacgagggacggctgagcctcggctacgccaccgacggcccttccacgcggcccccgctcgccgcggtctcccccttccaccggaacctggagaccgacgaacgctggctcgccctcaaggacacccccgccttcgccgcctaccgcctggccctgaactgcgcgtacctccacctcacccggctcggcctcacgcccgaccagcgcttcctgatctgccacctcgccgcgagcgccgccgacgacgtctaccgggccgtggcctcatgaccggggaacggtacgagctgcgcgccggcgtgcacctcgcccggctggacgcgggggtccgcctgttcgcctggccgcacgccgagaccctcggcgagctcacgcgggatgagctcgacctgctccgcgacctggccgccggaccccgcaccgccgggggaccgctcgtcgaacgcctgcgcgccggaggctggctcaagatcacgattacccgggacggcgtgcccctctacacgctcgccccgctgcgcccgccgccgccacggccggcggtgccggagcggccggttctgtccaggttcgccacgctgcggcgcgagggggacgcgctggtcgccggctccccgctggcctggtgcgacctgtacgtccacgaccccggcctggtcggcgacgtggtccggcccggcggcgaggtcctggccgacctggtgtgggccgggctggccgtgcccgaggaggccgaggaccgggagctgcggttccggcagtgggcgccgcacgagctggagttccatgaccggagccgggtggggcaccgcggctacctgggcgatgggttcggcgggacgttctgggcccgcggccggttcgacccgctgcccgcccggccccggccctaccccggccgggcgatcgagctgcccacgcccgacctggacgaactgcggctcaaggacccctcgctcacggacgtgctggagggccgcaggtccgtacgcgagcacgacgacgagcgcccgatcaccctcgacctgctcggcgagctgctgtaccgcacggccagggtgcgggagtgctggacggccgacggcgtcgaatacacccgcaagccgtacccctccggagggtccgtctacgagctggagatctaccccgtcgtcaggaacgtggccgggctcgaacccggcatgtaccactacgacgggcacgaccacgtgctcaggccggtacgtgaggccggccatcccgccgtacgccggctgctgaccgtcgccgggcacggcgccgccaccggccgccgcccccaggtcctgctcgcggtctccagccgcgccggccggctgctgtggaagtacgaaggcatggggtacgcgctgacgctcaagcacgtgggtgtgctctaccagacgctctactgcgtggccacggcgatgggcctggcggcctgcgggctcggcagcggcgactcggccgcgttcgccgaggcgaccgggcgggacccgttggaggagtgcgcggtcggcgaattcatgatcggaaccgcggaggcaccacgttgaacgacttcaaacccagactccccgcggacatacgcttcgcgcccacctccggcggtgtcgccttcctcacccgcgaaggcgtcgtgcggatgaacggggcctccgtccacctgtgggtggagcggctggccgtgcacctcgacgggagcaggaccgtggccgagctgaccgcggagctgcccgacgagcggcgggcgttcgtgacgagcctgctgaccacgctgcgcgagcgcgggctcgtggaggacgcgggagagcgcggcgtgctgcccgtggcggccggcaccgcgtgctcgctgctggccgacgcggaccggttcggcgagctggcgcccgccctggccgacgccctgcgcaggctggggctggaggtgctgacagagccgtcgggcggggcgctcgtgcacgtcgcggcggccgacgcggacgggctggccgccgccgggcgcctcgaccggctgagcgccgagcgcgggctgcccatcgcgcacgtgctgctgcgcgagggcgaggcgtggtggagcgagaccggcgtcgtcgggcgcgacgtcccgccgtgggcggccgggtggcggcggctggcggccatggaggccgtcccgcgtaccgccgggcacgtctccgtgtcgcccgtcgccgcggccgtggtcgccgggcaggtggtggcggacctggggcgctgcctgtccggaacgccccgcgtgtccggcggtccgcggctgcacgtggtggacctggacgggttcgggatgcggacgcactccttcctgccgcacctgtcggcgctgccgcccggcacgggggtgagcgcggggccgctcggcgaggaggagttctcgcggcgggcggccaggttgatggactcgcggacgggggtgttcgccgagatcgaggagggggagtacgggcagctcccgctgcacgtgtcggtgaccacggtggccgatccgatgggggccctcgggggtgcgctggcgcgggtgatcggcgtgggggcggacttcgccaccgcgcgataccgggcggcgcttaaggcactgaccgtgcacgggctgctcgccctcgacccccgccgcctggtcacggccgaggggacgcccctgaccggcgcgggggaacccgccagcggcttcccggctgatccgggcgcgagggaacggccgatcggtggcttttcggctgatccgggtgcgggtgagcggccggacgacggtctcctggacgacccgctggtgggcgacgtcgcggcgggacgggtcgaggcgttcgtgcgcggccgtgacctcgtcgagggcggtgaggtgctgctgcccgcccgcgcggtgttcctggatccgcacgccaccggggccgccgcggggtactcggccgaggaggcgctggtcgcggggttactggaccactgtgcggcactggccgggctcgacgggacgctgccgcccgtggacgtgggcggcgcgcgggcggccgacgacgagaccgcgcggtcggcggcgctgctggacgcgatcgggctgccgtaccaggtgctggacgccaccggcccgtcgggcgtgcccgtgcacgcggggacgctcggcgggcggctggtggcgcgggcggcgggcgtcacgcccctggccgcgctgcgggccacgctcgaagggatcctggccgcgtaccagggcattccgggccccgcacccctgcccgacctgtgcccggccgtcgcgcaggagccgccggtcgtcctcgacacgggggcggccgtgctcggcgccggtgagctcgccgccgccctggccggcgccgggctgcgggtcagcgccgttccgctggatcacgaccgggaggtacacgcgctcatgccgcacgtcgtccgcgtcgtcacggagccgctccatggctgaggtcgtggggacgggcctgctggccgaggagatcacgatcgcgctgaaggaccgcgacgggcgggcagaggtgacgatcaccgcctgcgacggctgggcgcgcggcccccggcgcgacggggcggcgtggttgcccgtgcgggtcgagtacggggtcgccgtcatcgggccgctggaacgccccggcgaacccggctgcgtgacctgcttcgagacccgccgggaccgggtcaggaccgacagcgccgatcgggccgccgtcctggccgtccacggccccgaactcgccgggcgggcctccccgtacctcggccgcctggccgcccgcacgatcgcggccctggccgcgcacgccgccgagggccccggatgcgccgtctggtacgtcgacctgcgcacgctcaccgccgaacgccacacgttcctgccggaaccccactgccccgactgcggcgacctgccggacgacgaccgcgcgtccgccgccttcgagcccggcgccgccccgcgacctcccggctaccgggtcaggaacgtgccggccgagcacggcgcgctgctcgacacgtacgtggacggcgaatgcgggctgatcaggcccctcgtccgggacacgctcggcggcctggtgatcgccggggcgatgctgccgctgcggttcgagggcggttcggagcccggcgtgggccgcacccgcggctaccgggccagcgaggtcaccgccgtgctcgaagcgctcgaacggtggggcggcgtcgagcccggcggcaagcgcacccgcgtgcgggcctcgtacgcggaggtggccgcggacgcgctcgaccctcgaacgctgggcgagcacgacacctcctcctacgagatggaggggttcgcgttccggccgttcgccgaggacgccgtctgcgactgggtgtgggggtactccttcgcccggcgctcgccgatcctcgtgccggagagcctggcctattactacgtacgggggcgggagcggccgttcctcttcgagatctcgaacgggtgcgcgctcggcggcagcatggcggaggccctgctctacgggctgctggagacggtcgagcgggacgcctttctcatgacctggtacgggcggctcccggtgccgcggatcgaccccggcacggcacgggaccgcaccatcccgctccaggccgccgcgatcatcgcggagaccggttatcgggtggagctgtacgacacgacgatggagcacggcatccccagcgtgtgggcgatgggcgtgcggcaggacggcgatccgggcaggccgcgcctggtgtgcgccgccggggcccacctggtgccggagaaggccgtgctcagcgcgctgagcgagctggggccgctgctggccgacctgatccggcggtatccggacgaggtgcaccgagcccaggagatggtgacaaatcctgatctggtggcgacgatgcacgaccactccaccctgtacggcgccgacgcggccttcgaccggctctcgttcctgaccggcggcacgggcgtgcgggggctgcccgacatggaggcgttcaccgtgcccggtgatctcgacgtcatgagggtggtggaccgcttcctcgccgaggggatggacgtcgtcgtcatcgaccagaccacgccggagcatcgggcccgcggtttctcctgcgtcaaggtgctggtgcccggcacgctcccgatgaccttcggccaccgcaaccgcagggtgcggggcctgacccgcccgctggagctgccgtaccggctcggccaccgcgacacgcccctgacccccgccgacctgaaccccgacccacaccccttcccatgaccaccgagccgtttcccgcgccgacggggccgcccgcagcgccgacggggccgtctcccgcgctggcggagccgtctgctgcatcgcccggcccgttgtccgttcggcaggggccgtctcctgtgaccgccgagctgcgtgacctggcgatcttccgtgccgatctgcatgcccaccccgagctgtcctggcaggaggccaggacggccgatcgcctggcggggcggctggcggcggccgggtacgaggtcacgaccgggatgggcggtcatggggtggtcgggaggctgtgccgtggcgacggggtcacggtcatgctcagagccgagctggacgccctccccgtcaaggaggagaccggcctgtcatacgccagcaccgccaccgccacgaccctcgacggcaggacggtgccggtctcccacgcctgcggccacgacctccatctcgcctgcctggtcggcgccgcccggcggctggccgcctgcgacgactggcggggcacggtgctcgtgatcggccagcccgccgaggagacgctggagggcgccgccgcgatgctcgccgacggcctgtacgagcggttcggcgtcccggacgtggcgctggcccagcacgtctcgccgttccccgcgggcctgatcgcctacccggagccgcccaccgcggccggcgccgaactccgcgtcgtcgtcacgggggacggcggccacgtcggagacatcggccgggctggtcacgtcgatcgagccgtggggcgcaatccggtcgcggccgtcgccgcgctcgttcaccggctcgatcagacggcgttcgaccaggcgatcgtcaccgtggggacgttgcacgcgggcgagcgcgccaacgtgatccccaccctcgccgaggccgggatcacggtcagggccgcgacggaggaggcggtgacccgggccgtcgcccgcgtcgcccggctggcggaggagacggcgggagccggcgtcatcgtggtctccagggtgccgcccggcgtgaacgaccccgccgccaccgccctggtacgccgggcccacgaggccgcgctcggcgcggtcgtcaccgcccccggcggctcggcctgcgaggacttccccctctacggggtgccctccgtctactggtacgtgggcgcggccccacccgccggcctggtcggacgcccgcataccgggactttccgccccgatcccgtgcccacgttgagggcaggggtcaccgccatgcaaacggctgccctggcagtccttgcgaacgcgtcacagttcgcccctccatcccgatatcatggacccggagcagtagccgagcactga

SEQ ID NO: 64 - protein, 60 aa, nonomuraea sp.
MSNASIGQEIGVEGLTGLDVDALEISDYVDETLLDGEDLTVTMIASASCTTCICTCSCSS

SEQ ID NO: 65 - DNA, 180 bp, nonomuraea sp.
atgagtaatgcctccatcggtcaggagatcggcgtcgagggcctgacgggcctggacgtcgacgctctggagatcagcgactacgtggacgagacgctgctcgacggcgaggacctcacggtgacgatgatcgcctccgcctcgtgcaccacctgcatctgcacgtgcagctgcagctcc